(19) United States
(12) Patent Application Publication
(10) Pub. No.: US 2021/0332489 A1
(43) Pub. Date: Oct. 28, 2021

(54) LASER-INDUCED GRAPHENE ELECTRODES ADAPTABLE FOR ELECTROCHEMICAL SENSING AND CATALYSIS

(71) Applicant: Iowa State University Research Foundation, Inc., Ames, IA (US)

(72) Inventors: Jonathan Claussen, Ames, IA (US); Carmen L. Gomes, Ames, IA (US); Raquel Rainier Alves Soares, Ames, IA (US); Robert Hjort, Ames, IA (US); Cicero Cardoso Pola, Ames, IA (US)

(21) Appl. No.: 17/302,229

(22) Filed: Apr. 27, 2021

Related U.S. Application Data
(60) Provisional application No. 63/016,068, filed on Apr. 27, 2020.

(51) Int. Cl. C25B 11/043; G01N 27/30; C01B 32/205; C01B 32/184

(52) U.S. Cl. CPC C25B 11/043; G01N 27/308; C01B 32/205; C01B 32/184; C01B 2204/24; C01P 2002/85; C01P 2006/40; C01P 2004/03; C01P 2002/72; C01P 2002/82; C01B 2204/04

(57) ABSTRACT

Apparatus and methods of fabrication and use of highly effective laser-induced graphene (LIG) electrodes including for electrochemical sensing and catalysis. One example is a sensitive and label-free laser-induced graphene (LIG) electrode functionalized for a specific application. One example of functionalization with antibodies, an enzyme, or an ionophore to electrochemically quantify a target species The LIG electrodes were produced by laser induction on film having a carbon precursor (e.g. polyimide) in ambient conditions, and hence circumvent the need for high-temperature, vacuum environment, and metal seed catalysts commonly associated with graphene-based electrodes fabricated via chemical vapor deposition processes. These results demonstrate how LIG-based electrodes can be used for electrochemical sensing in general. Other examples of applications include, but are not limited to, ion-sensing, pesticide monitoring and detection, and water splitting, using the LIG-based electrode(s) adapted for those purposes.

### CROSS-REFERENCE TO RELATED APPLICATION

This application claims the benefit of Provisional Application U.S. Ser. No. 63/016,068 filed on Apr. 27, 2020, all of which is herein incorporated by reference in its entirety.

### GOVERNMENT RIGHTS

This invention was made with Government support under Grant Nos. CBET1706994 and CBET1756999 awarded by the National Science Foundation and Grant Nos. 2020-67021-31375 and 2021-67021-34457 awarded by the National Institute of Food and Agriculture of the US Department of Agriculture. The government has certain rights in the invention.

### I. BACKGROUND OF THE INVENTION

### A. Field of the Invention

The present invention relates to apparatus, methods, and systems of making and utilizing electrical and electronic circuits and components and, in particular, in the context of graphene-based electronics and, further, in the context of laser induced graphene (LIG) electrodes that can be used for various applications by effective functionalization of portions of the LIG electrodes. Examples of applications include but are not necessarily limited to electrochemical sensing, catalysis, immunosensors, electrochemical ion sensing, pesticide monitoring, and water splitting.

### B. Problems in the State of the Art

Graphene-based electronics offer great promise for a wide variety of applications including supercapacitors and batteries, graphene tattoo sensors, and other electronics [1-13]. Challenges with realizing graphene-based electronics lie in relatively complex fabrication procedures, which have generally included chemical vapor deposition (CVD) and/or complex substrate-transfer techniques [14]. However, alternative scalable manufacturing protocols for graphene-based electrical circuits are beginning to emerge, including solution-phase printing (inkjet printing [15, 16], screen printing [17], dispenser printing [18]) and direct laser scribing among others [9, 10]. A promising alternative to printed graphene circuits is laser-induced graphene, which is a one-step lithography-free process upon which a laser converts sp³-hybridized carbon found in substrates, such as polyimide, into sp²-hybridized carbon: the carbon allotrope found in graphene [10, 19, 20]. Laser induced graphene has been used to prevent microbial fouling and in antimicrobial applications [21]. It has also been used for biosensing applications based on ion selective membrane and enzymatic and aptamer binding reactions [4, 14, 22]. However, a laser-induced graphene immunosensor has not been demonstrated.

Biosensors based on impedimetric detection have been designed for a number of pathogens, including Salmonella Typhimurium [23-26], Escherichia coli O157:H7 [27], Listeria monocytogenes [28], and E. coli O111[29], to name a few. The main challenge in the field of biosensing for monitoring pathogens is not response time, but rather the poor detection limit (approximately 10²-10³ CFU mL⁻¹). To resolve this, most groups have integrated a pre-enrichment step. Although this indeed improves detection limit when only considering signal acquisition, in applied settings this approach trivializes the need for the rapid sensor (the incubation step can take as long as a rtPCR assay). This invention resolves this problem by optimizing the bacteria capture efficiency by developing high surface LIG-based sensors.

This work demonstrates the fabrication of a highly porous, high resolution, thin film (film thickness of ˜25 nm) laser-induced graphene on a polyimide sheet and its application to create in-field electrochemical immunosensor detection of foodborne pathogen, Salmonella enterica. Previous studies have demonstrated highly sensitive and label-free sensors, for example those based on electrodes made of gold nanoparticles[36] and double-walled electrode made of carbon nanotubes[37], but all required time greater than 22 minutes (pre-enrichment step) to perform the test. These steps add complexity to the assay and render the technique difficult for point-of-use applications. Furthermore, the published biosensors that display perfounance similar to this invention use expensive or complex fabrication techniques including chemical vapor deposition or precious metal [24, 25, 38], significantly affecting the device's cost and consequently commercialization.

Both the use of metallic particles or need of pre-treatment of the sample (pre-labeling or pre-enrichment) increase the cost of the biosensor, as compared to the LIG immunosensor which can be used as a one-time, disposable biosensor. In this invention, we demonstrate that this graphene electrode fabrication technique using laser inducing process is capable of sensing Salmonella enterica at low concentration, 13±7 CFU mL⁻¹ in complex media, chicken broth with a response time of around 20 minutes over a broad range of bacteria concentration, from 10¹ to 10⁵ CFU mL⁻¹ without the need to pre-label or pre-enrichment the sample nor the need to immobilize metallic nanoparticles onto the graphene surface to increase its reactive surface area.

### References [Indicated in Brackets in Background of the Invention Section, Supra]

1. Ferrari, A. C., et al., Science and technology roadmap for graphene, related two-dimensional crystals, and hybrid systems. Nanoscale, 2015. 7(11): p. 4598-4810.
2. Novoselov, K. S., et al., A roadmap for graphene. Nature, 2012. 490(7419): p. 192-200.
3. Kurra, N., et al., Laser-derived graphene: a three-dimensional printed graphene electrode and its emerging applications. Nanotoday, 2019. 24: p. 81-102.
4. Fenzl, C., et al., Laser-Scribed Graphene Electrodes for Aptamer-Based Bio.sensing. ACS Sensors, 2017. 2: p. 616-620. [incorporated by reference herein].
5. Nayak, P., et al., Highly Efficient Laser Scribed Graphene Electrodes for On-Chip Electrochemical Sensing Applications. Advanced Electronic Materials, 2016: p. 1600185.
6. Nayak, P., et al., Monolithic laser scribed graphene scaffolds with atomic layer deposited platinum for the hydrogen evolution reaction. Journal of Materials Chemistry A, 2017. 5: p. 20422.
7. Kabiri Ameri, S. D. Akinwande, and N. Lu, Graphene Electronic Tattoo Sensors. ACS Nano, 2017. 11(7634-7641).
8. Jang, H., et al., Graphene-based flexible and stretchabable electronics. Adv. Mater., 2016. 28(4184-4202).
9. El-Kady, M. F., et al., Laser scribing of high-performance and flexible graphene-based electrochemical capacitors. Science, 2012. 335: p. 1326-1330.
10. Lin, J., et al., Laser-induced porous graphene films from commercial polymers. Nat. Commun., 2014. 5: p. No. 5714. [incorporated by reference herein].
11. Ye, R., D. K. James, and J. M. Tour, Laser-Induced Graphene. Accounts of Chemical Research, 2018. 51(7): p. 1609-1620.
12. Chyan, Y., et al., Laser-Induced Graphene by Multiple Lasing: Toward Electronics on Cloth, Paper, and Food ACS Nano, 2018. 12(3): p. 2176-2183.
13. Li, L., et al., High-Performance Pseudocapacitive Microsupercapacitors from Laser-Induced Graphene. Advanced Materials, 2016. 28(5): p. 838-845.
14. Garland, N. T., et al., Flexible Laser-Induced Graphene for Nitrogen Sensing in Soil. ACS Applied Materials & Interfaces, 2018. 10(45): p. 39124-39133.
15. Das, S. R., et al., 3D Nano-structured Inkjet Printed Graphene via UV-Pulsed Laser Irradiation Enables Paper-Based Electronics and Electrochemical Devices. Nanoscale, 2016. 8(35): p. 15825-16074. [incorporated by reference herein].
16. He, Q., et al., Enabling Inkjet Printed Graphene for Ion Selective Electrodes with Posiprint Thermal Annealing. ACS Appl. Mater. Interfaces, 2017. 9(14): p. 12719-12727.
17. Huang, X., et al., Highly flexible and conductive printed graphene for wireless wearable communications applications. Sci. Rep., 2015. 5: p. No. 18298.
18. Eu, K., et al., Graphene oxide-based electrode inks for JD-printed batteries. Adv. Mater., 2016. 28(2587-2594).
19. Ye, R., et al., In situ formation of metal oxide nanocrystals embedded in laser-induced graphene. ACS Nano, 2015. 9: p. 9244-9251.
20. Smith, M. K., et al., Thermal conductivity enhancement of laser induced graphene foam upon P3ht infiltration. Appl Phys. Lett., 2016.109: p. No.
253107.
21. Singh, S. P., et al., Laser-Induced Graphene Layers and Electrodes Prevents Microbial Fouling and Exerts Antimicrobial Action. ACS Applied Materials & Interfaces, 2017. 9(21): p. 18238-18247.
22. Vanegas, D. C., et al., Laser scribed graphene biosensor for detection of biogenic amines in food Samples using locally sourced materials. Biosensors, 2018. 8(2): p. 42.
23. Labib, M., et al., Aptamer-based impedimetric sensor for bacterial typing. Analytical Chemistry, 2012. 84: p. 8114-8117.
24. Farka, Z., et al., Rapid Immunosensing of Salmonella typhimurium Using Electrochemical Impedance Spectroscopy: the Effect of Sample Treatment. Electroanalysis, 2016. 28(8): p. 1803-1809.
25. Pagliarini, V., et al., Treated Gold Screen-Printed Electrode as Disposable Platform for Label-Free Immunosensing of Salmonella typhimurium. Electrocatalysis, 2019. 10(4): p. 288-294,
26. Xu, M., R. Wang, and Y. Li, Rapid detection of Escherichia coli at OJ 57:H7 and Salmonella typhimurium in foods using an electrochemical immunosensor based on screen printed interdigitated microelectrode and immunomagnetic separation. Talanta., 2016. 148: p. 200-208.
27. Wu, W., et al., An aptamer-based biosensor for colorimetric detection of Escherichia coli 0157:H7. Plosone, 2012. 7(11): p. 48999,
28. Ohk, S. H., et al., Antibody-aptamer functionalized fiber-optic biosensor for specific detection of Listeria monocytogenes from food. Journal of Applied. Microbiology, 2010. 109: p. 808-817.
29. Luo, C., et al., A rapid and sensitive aptamer-based electrochemical biosensor for direct detection of Escherichia coli 0111. Electroanalysis, 2012. 24(5): p. 1186-1191.
30. Hondred, J. A., et al., Printed Graphene Electrochemical Biosensors Fabricated by inkjet Maskless Lithography for Rapid and Sensitive Detection of Organophosphates. ACS Applied Materials & Interfaces, 2018. 10(13): p. 11125-11134. [incorporated by reference herein].
31. Sidhu, R., et al. Impedance biosensor for the rapid detection of Listeria spp. based on aptamer functionalized Pt-interdigitated microelectrodes array. in SPIE Commercial+ Scientific Sensing and Imaging. 2016. International Society for Optics and Photonics.
32. Lian, Y., et al., A new aptamer/graphene interdigitated gold electrode piezoelectric sensor for rapid and specific detection of Staphylococcus aureus. Biosensors and Bioelectronics, 2015. 65: p. 314-319.
33. Kim, Y. S., J. H. Niazi, and M. B. Gu, Specific detection of oxytetracycline using DNA aptamer-immobilized interdigitated array electrode chip. Analytica Chirnica Acta, 2009. 634(2): p. 250-254.
34. Zou, Z., et al., Functionalized nano interdigitated electrodes arrays on polymer with integrated microfluidics for direct bio-affinity sensing using impedimetric measurement. Sensors and Actuators A: Physical, 2007. 136(2): p. 518-526.
35. Varshney, M. and Y. Li, Interdigitated array microelectrodes based impedance biosensors for detection of bacterial cells. Biosensors and Bioelectronics, 2009. 24(10): p. 2951-2960.
36. Silva, N. F. D., et al., In situ formation of gold nanoparticles in polymer inclusion membrane: Application as platform in a label-free potentiometric innnunosensor for Salmonella typhimurium detection. Talanta, 2019. 194: p. 134-142.
37. Punbusayakul, N., et al., Label-free as-grown double wall carbon nanotubes bundles for Salmonella typhimurium immunoassay. Chem. Cent. J., 2013. 7: p. 1-8.
38. Alexandre, D. L., et al., A Rapid and Specific Biosensor for Salmonella typhimurium Detection in Milk. Food and Bioprocess Technology, 2018. 11(4): p. 748-756. [incorporated by reference herein].

Similar and additional issues have been identified regarding the state of the art of electrode-based technologies, including electrochemical ion sensing, pesticide monitoring, and water-splitting.

### II. SUMMARY OF THE INVENTION

### A. Objects, Features, and Advantages

This invention introduces a laser-induced graphene electrodes that can be adapted for various useful purposes. A laser is controlled to efficiently and effectively create LIG patterns from a substrate of carbon precursor. The controlled operation of a laser generates LIG patterns which have characteristics and properties that are shown to produce advantageous results as LIG electrodes in a variety of effective ways. The combination of the controlled LIG generation and specific functionalization of at least part of the generated LIG pattern have been demonstrated effective for different applications, inter alia, electrochemical sensing, catalysis, immunosensing, ion sensing, pesticide monitoring, and water splitting.

In one example, the combination is used in biochemical sensing including immunosensing. More specifically this invention demonstrates for the first time that LIG has been functionalized with antibodies for antibody-based electrochemical biosensing (immunosensing). Details regarding this major point is given below.
1. LIG. While graphene-based biosensors and immunosensors are not new and have been used for electronic applications, all the previous works choose to deposit graphene oxide and then reduce it to yield reduced graphene oxide (some papers call this “functionalized graphene”). Conversely, in one embodiment, our graphene electrodes are fabricated using a simple method dubbed laser induced graphene (LIG) which is a direct write method that can be used in a one-step process to convert carbon sp³ into carbon sp² under high temperature by laser induction, which creates porous graphene, as shown by Raman spectroscopy and Scanning Electron Microscopy Imaging. This would have benefits in higher EIS signal.
2. Best limit of detection and selectivity for an immunosensor that can monitor, for example, Salmonella enterica in food sample. The immunosensor was able to detect the pathogen at low concentration, 13±7 CFU mL⁴ in complex media, chicken broth with a response time of around 20 minutes. Electrochemical impedance spectroscopy was used as a label-free detection over a broad range of bacteria concentration, from 10¹ to 10⁵ CHI mL⁴.
3. Best limit of detection and selectivity for an immunosensor that can monitor Salmonella enterica in an actual food sample that avoid bacterial enrichment. Most immunosensors that have been developed for Salmonella enterica are validated in PBS or water and not actual food samples and the response time is higher than 20 minutes, with detection limits higher than 13 CFU mL⁻¹ Commercially available immunosensors require an enrichment step that can take up from 18-24 h to obtain results. Many immunosensors need to label the antigen with a redox probe (e.g., metallic nanoparticle) or fluorescent label to improve the electrochemical signal sensitivity or visualize the biorecognition agent binding event. Other biosensors require steps to preconcentrate the target analyte before the biosensor can make a readable measurement, increasing the response time. Such labeling and preconcentration steps significantly increases assay complexity and are not amenable to in field experiments and are generally difficult to perform at the point-of-use. This invention does not require these.

The laser inducing and biofunctionalization are amenable to scalable manufacturing. This is primarily due to the lack of need for preconcentration and labeling steps makes the biosensor low-cost and well-suited for one-time, disposable biosensing.

Other examples include electrochemical ion-sensing, pesticide monitoring, catalysis, and water-splitting, as will be discussed further herein.

### B. Aspects of the Invention

Aspects of the invention include apparatus, methods, and systems utilizing functionalized LIG-based patterned electrodes for a variety of beneficial purposes.

One is sensors for electrochemical detection. Another is sensors for ionic species of interest. Another is sensors for monitoring for presence of a pesticide of interest. Another is energy generation by water splitting. An aspect of the invention is the combination of a specifically controlled laser for generation of a particular LIG pattern or patterns with effective use of the LIG pattern or patterns for a variety of purposes.

In the example of biosensing, including as an immunosensor, apparatus, methods, and systems according to the invention combine an LIG-based pattern which is effective for functionalization with relevant binding agents or anti-bodies, and then capture and measurement for target species or pathogens.

In the example of ion sensing, apparatus, methods, and systems according to the invention combine LIG patterns which are effective for functionalization to capture ionic species for detection and measurement.

In the example of pesticide monitoring, apparatus, methods, and systems according to the invention combine LIG patterns which are effective for functionalization to detect presence of a pesticide of interest in a sample or in situ. In the example of water-splitting, apparatus, methods, and systems according to the invention combine LIG patterns which are effective for functionalization as water-splitting electrodes.

Methods according to the invention include use of the foregoing apparatus for electrochemical detection. In addition, methods include direct or indirect writing of an effective LIG pattern(s) by selection and control of the operating parameters of a laser. Methods according to the invention including a combination of ordered steps to fabricate LIG electrodes and functionalize them as above.

Systems according to the invention include foregoing apparatus or methods in operative connection to other circuits or components. For example, electrochemical reading and analysis components are operatively connected to functionalized LIG electrodes according to aspects of the invention for use as bio-sensing, immunosensing, and pesticide monitoring. Electrical/electronic circuits and components are operatively connected to functionalized LIG electrodes according to aspects of the invention for ion sensing and water-splitting, The systems can include components for rapid and effective use on-site or for in-field sensing.

### III. BRIEF DESCRIPTION OF THE DRAWINGS

FIG. 1 is a high-level generalized diagram of an LIG electrode according to an exemplary embodiment of the invention.

FIG. 2 is a high-level generalized diagram of a method of making an LIG electrode and then functionalizing it according to an exemplary embodiment of the invention.

FIG. 3 is a graph showing proof of concept of the method of FIG. 2.

FIG. 4 is a high-level generalized diagram of a system according to an exemplary embodiment of the invention using a LIG electrode.

FIGS. 5 and 6 are additional diagrams of a method and system according to an exemplary embodiment of the invention, including the ability to laser-scribe any of a number of UG patterns to form an LIG electrode, and then biofunctionalize the LIG electrode, and then have a field-use or point-of-use hand-held, portable reader, and optionally communicate wirelessly with further devices.

FIG. 7 is a diagram and results of using LIG to form complex LIG patterns on an appropriate substrate.

FIGS. 8 and 9 are high level diagrams showing how an electrode can be biofunctionalized.

FIG. 10 is a chart illustrating proof-of-concept of efficacy of immunosensors according to the invention which are frozen for later use.

FIGS. 11 A-C. Fabrication, biofunctionalization, and sensing scheme of an LIG immunosensor according to an exemplary embodiment of the invention. Fabrication and biofunctionalization steps included: FIG. 11A shows at (a) LIG processing onto a polyimide (Kapton) sheet to create; (b) a working electrode; (c) passivation of working electrode with lacquer; and (d) an SEM image showing an LIG surface. FIG. 11B shows at (e) biofunctionalization with Salmonella antibodies immobilized on the working electrode via carbodiimide cross-linking chemistry. FIG. 11C shows at (f) Salmonella binding to the electrode and the resultant Nyquist plot generated during electrochemical sensing.

FIGS. 12A-F. SEM images of the bare LIG electrode of FIGS. 11A-C at 5 kV: FIG. 12A shows 230× and FIG. 12B shows 2000× magnification, respectively, confirming the porous graphene morphology; FIG. 12C shows a SEM cross-sectional image of the same electrode at 5 kV and 2300× magnification; FIG. 12D shows an EDS spectrum of the LIG-electrode, showing the predominance of carbon and a small portion of oxygen, indicating the change in chemical composition and chemical bonds after laser processing; FIG. 12E shows a representative XRD spectrum comparing polyimide (PI) and LIG, which displays a peak at 2θ=26.5°, indicating graphitization; and FIG. 12F shows a Raman spectrum comparing PI and LIG showing the three characteristic peaks of graphene D, G and 2D with a ratio I_{2D}/I_{G}˜10.35, which indicates multi-layer graphene formation.

FIGS. 13A-C. FIG. 13A shows a representative cyclic voltammogram of an LIG-electrode according to an exemplary embodiment of the invention vs. Ag/AgCl in 0.1 M KCl containing 4 mM K₃[Fe(CN)₆]/K₄[Fe(CN)₆] at scan rates from 50 to 200 mV FIG. 13B shows a Cottrell plot of the LIG-electrode vs. Ag/AgCl in the same solution at scan rates 50, 75, 100, 125, 150 and 200 mV s⁻¹ with corresponding values of ESA=0.104±0.032 cm² and k₀=0.0146±0.0031 cm s⁻¹; and FIG. 13C shows an optimization of antibody concentration showing no significant (p>0.05) difference to R_{ct} variation according to Tukey-HSD test. Plot b) was generated from graph a) using a method described herein.

FIG. 14A-D. Representative Nyquist plots of impedance spectra of an immunosensor according to an exemplary embodiment of the invention for increasing concentration of Salmonella enterica at: FIG. 14A shows 0 CFU mL⁻¹ (red), 29 CFU ML⁻¹ (orange), 63 CFU mL⁻¹ (yellow), 96 CFU mL⁻¹ (green), 512 CFU mL⁻¹ (blue), and 957 CFU mL⁻¹ (purple) in BPW; inset shows equivalent Randles-Ershler circuit used to fit the curves and to calculate the R_{ct}; FIG. 14B shows 0 CFU mL⁻¹ (brown), 33 CFU mL⁻¹ (red), 92 CFU mL⁻¹ (orange), 444 CFU mL⁻¹ (yellow), 923 CFU mL⁻¹ (green), 10⁴ CFU mL⁻¹ (blue), and 10⁵ CFU mL⁻¹ (purple) in chicken broth; bacteria concentrations were confirmed by plate counting; inset shows equivalent Randles-Ershler circuit used to fit the curves and to calculate the Rct. Linear calibration curve of charge transfer resistance change (ΔR_{ct}) versus Salmonella enterica concentrations (log CFU mL⁻¹) in BPW (FIG. 14C) showing a linear regression corresponding to ΔRct (%)=8 (concentration of bacteria)+0.007 with R²=0.984; and in chicken broth (FIG. 14D) with a linear regression corresponding to ΔR_{ct} (%)=4 (concentration of bacteria)+0.023 with R²=0.989; data shown as mean±SD, n=3. Plots c) of FIG. 14C and d) of FIG. 14B were generated from graphs a) of FIG. 14A and b) of FIG. 14B; respectively.

FIGS. 15A-B. FIG. 15A shows percentage charge transfer resistance change (ΔR_{ct} %) versus a constant concentration (10⁴ CFU mL⁻¹) of different interferent bacteria and Salmonella enterica Typhimurium to show specificity of the immunosensor of FIGS. 14A-D. A significant change (p<0.05) in ΔR_{ct} (%) was observed when Salmonella enterica Typhimurium was evaluated (n=3). Bacteria concentrations were confirmed by plate counting FIG. 15B shows stability of the immunosensors during shelf life test for 7 days. Mean values presenting the same lowercase letter are non-significantly different considering a level of significance of 5%. Error bars represent standard error calculated from three repetitions.

FIG. 16. FIG. 16 is a schematic of fabrication, functionalization of the LIG HRP sensors, LIG ISE sensors and water splitting electrodes according to additional exemplary embodiments of the invention.

FIG. 17A-F. FIG. 17A is a SEM image of an LIG produced according to FIG. 16; FIG. 17B is a SEM Image of an NiO-LIG produced according to FIG. 16, and FIG. 17C is an SEM image of a Pt-LIG electrode produced according to FIG. 16, all at scale bar 5 μm. FIG. 17D is a Ramen Spectrum of the LIG of FIG. 17A. FIG. 17E is an XPS of Ni 2p of FIG. 17B, and FIG. 17F is an XPS of the Pt 4f regions in FIG. 17C.

FIGS. 18A-D. FIG. 18A is cyclic voltammograms of 11 bare LIG electrodes of FIG. 16 in ferricyanide-ferrocyanide mixture. Voltammetry settings: initial potential +0.4 V, low potential 0 V, scan rate 10 mV/s, sample interval 0.001 V, sensitivity 1e-4 A/V. FIG. 18C is a real signal of the K⁺ ISE (blue line) and bare LIG electrode (red line) of FIG. 16 to the sequential additions of KCl (from 10⁻⁶ to 10⁻¹ M, with 10^{-0.5} step). FIG. 18D is a typical calibration curve of the K⁺ ISE of FIG. 16. FIG. 18C is a typical calibration curve of the pH ISE of FIG. 16.

FIGS. 19A-C. FIG. 19A is an atrazine calibration curve for a sensor according to FIG. 16. FIG. 19B is an electrode sensitivity to Atrazine for such a sensor. FIG. 19C is an interference test of select herbicides for such a sensor.

FIGS. 20A-D. FIG. 20A is an LSV of LIG-Pt according to FIG. 16 for HER and FIG. 20B is a LIG-NiO for OER at a scan rate of 5 mV/s with corresponding Tafel slope for a LIG-Pt (FIG. 20C) and LIG-NiO (FIG. 20D).

FIGS. 21A-G. Schematics of the fabrication process of an exemplary embodiment according to the invention for pesticide detection, namely, FIG. 21A, step a for a CO₂ laser on bare PI before laser scribing; FIG. 21A step b for CO₂ laser scribing; FIG. 21A step c for resultant laser induced graphene; FIG. 21A step d for application of acrylic polish as passivation layer on electrode stem and silver paste application on electrode contact pad; and FIG. 21A step e an actual image of an LIG according to this embodiment. Characterization of the laser induced graphene electrode, namely, FIGS. 21B-C are SEM images in different magnifications; FIG. 21D are Raman spectra; FIG. 21E is an XPS survey; FIG. 21F is cyclic voltammograms at various scan rates in 5 mM ferri/ferrocyanide; and FIG. 21G is a Randles-Sevcik plot.

FIG. 22: Diagram of chemical structure of different neonicotinoids with the nitroguanidine functional group highlighted in red.

FIGS. 23A-F: Square wave voltammogram and inset calibration plots for: FIG. 23A imidacloprid; FIG. 23B clothianidin; FIG. 23C thiamethoxam; FIG. 23D dinotefuran; FIG. 23E dinotefuran repeatability; and FIG. 23F combined neonicotinoid model.

FIGS. 24A-B. Interference data including: FIG. 24A square wave voltammograms and FIG. 24B current change comparison.

FIGS. 25A-F. Supplemental information for the water-splitting example of FIG. 16 including SEM image of the LIG (FIG. 25A); NiO-LIG (FIG. 25B) and Pt-LIG electrode (FIG. 25C), scale bar 5 Ramen Spectrum of LIG (FIG. 25D). The XPS of Ni 2p (FIG. 25E) and Pt 4f (FIG. 25F) regions.

FIGS. 26A-B. Supplemental information for the water-splitting example of FIG. 16 including an EDS element mapping of a NiO-LIG electrode (FIG. 26A) with corresponding spectrum (FIG. 26B).

FIGS. 27A-B. Supplemental information for the water-splitting example of FIG. 16 including an EDS element mapping of a Pt-LIG electrode (FIG. 27A) with corresponding spectrum (FIG. 27B).

FIGS. 28A-D. Supplemental information for the water-splitting example of FIG. 16 including LSV of LIG-Pt for HER (FIG. 28A) and LIG-NiO for OER at a scan rate of 5 mV/s (FIG. 28B) with corresponding Tafel slope for LIG-Pt (FIG. 28C) and LIG-NiO (FIG. 28D).

The following are incorporated by reference herein as if fully a part thereof and supplement this description at least as follows:

Background information about certain state-of-the-art practices by others can be found at the following, each of which is incorporated by reference herein: Cardoso, et al. Molecularly-imprinted chloramphenicol sensor with laser-induced graphene electrodes. Biosensors and Bioelectronics 124-125 (2019) 167-175; and Fenzl, et al. Laser-Scribed Graphene Electrodes for Aptamer-Based Biosensing. ACS Sens. 2017, 2, 616-620; and Hong et al. Direct-laser-writing of three-dimensional porous graphene frameworks on indium-tin oxide for sensitive electrochemical biosensing. Analyst, 2018, 143, 3327-3334.

Background information about functionalization of biosensors with binding agents can be found at the following, each of which is incorporated by reference herein: U.S. Patent Application Publication US2019/0330064 A1 (J. Tour, et al.); and U.S. Patent Application Publication US2020/00002174 A1 (J. Tour, et al.).

Background information about interdigitated electrodes fabrication, functionalization with antibodies, and sensing using a potentiostat can be found at the following, each of which is incorporated by reference herein: U.S. Pat. No. 5,958,791 (M. Roberts, et al.); and U.S. Patent Application Publication US2018/0059101 A1 (S. MacKay et al.).

### IV. DETAILED DESCRIPTION OF EXEMPLARY EMBODIMENTS OF THE INVENTION

### A. Overview

For a better understanding of the invention and its aspects, non-limiting examples of some of the ways and forms those aspects can be made, used, and practiced will now be set forth in detail. It is to be understood these are examples only, and that these examples are neither inclusive nor exclusive of all forms and embodiments the invention can take.

These embodiments will be discussed primarily in the context of sensing of pathogenic bacteria in food samples. As will be appreciated by those skilled in this technical area, aspects of the invention can be applied in analogous ways to other biochemical analytes in non-food samples, for example, water quality, medical sensing applications, agricultural sensing applications.

These embodiments will be discussed primarily in the context of LIG electrodes. As will be appreciated by those skilled in this technical area, aspects of the invention can be applied in analogous ways to other patterns of LIG, for example, interdigitated electrodes (IDE), dipstick electrodes, serpentine electrodes, all-in-one electrodes (working, counter, and reference electrodes in the same device). As further discussed infra, aspects of the invention can be applied to a variety of working applications. These include but are not necessarily limited to electrochemical immunosensing, electrochemical ion sensing, pesticide monitoring, water-splitting, and electrochemical pesticide sensing, including neonicotinoids and others.

### B. Generalized Embodiment

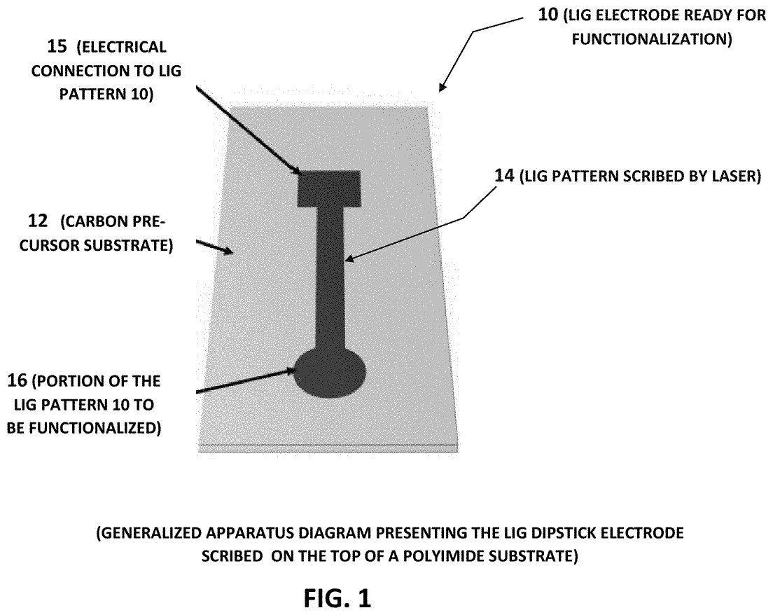

With reference to FIGS. 1-10, at a generalized level, aspects of the invention are illustrated. As will be appreciated with reference to the Specific Embodiments and Examples infra., aspects of the invention pertain to at least the following.

As illustrated in FIG. 1, an LIG electrode 10 according to aspects of the invention can include a LIG-based pattern 14 generated from a suitable starting substrate 12. The pattern 14 is shown diagrammatic in the sense it is intended to illustrate one of the versatilities of use of a laser for LIG is that the beam width and movement relative a surface can be controlled with high resolution but in virtually an infinite number of faun factors. A simple working electrode 10 as in FIG. 1 is one example. Its LIG pattern 14 on substrate 12 in this example has portion 15 for connection by known techniques to sensor circuit (not shown in FIG. 1) and a portion 16 that can be functionalized.

In some applications, pattern 14 is a single continuous pattern such as the dip-stick type pattern in FIG. 1. In some applications, pattern 14 can be replicated one or more times on the same substrate 12. In some applications, pattern 14 can be replicated on different substrates or diced from the same substrate to create two or more stand-alone LIG electrodes. Those stand-alone electrodes can be used separately or together.

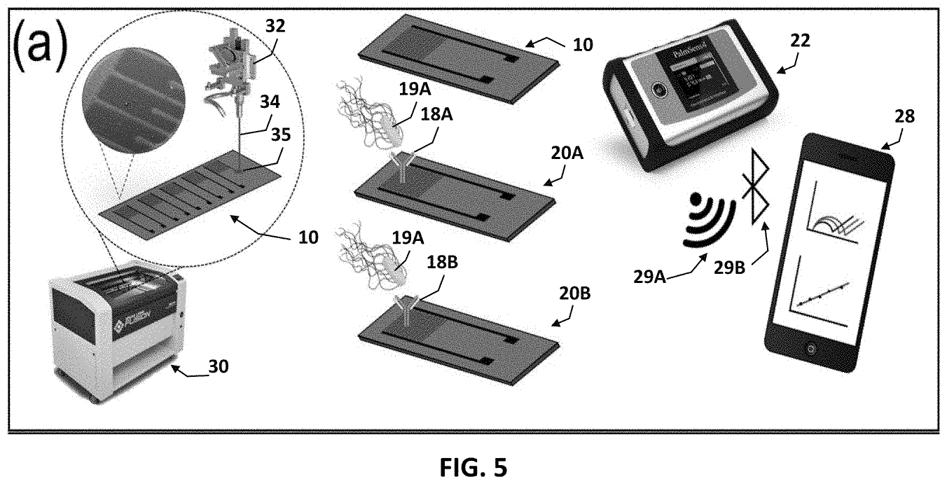

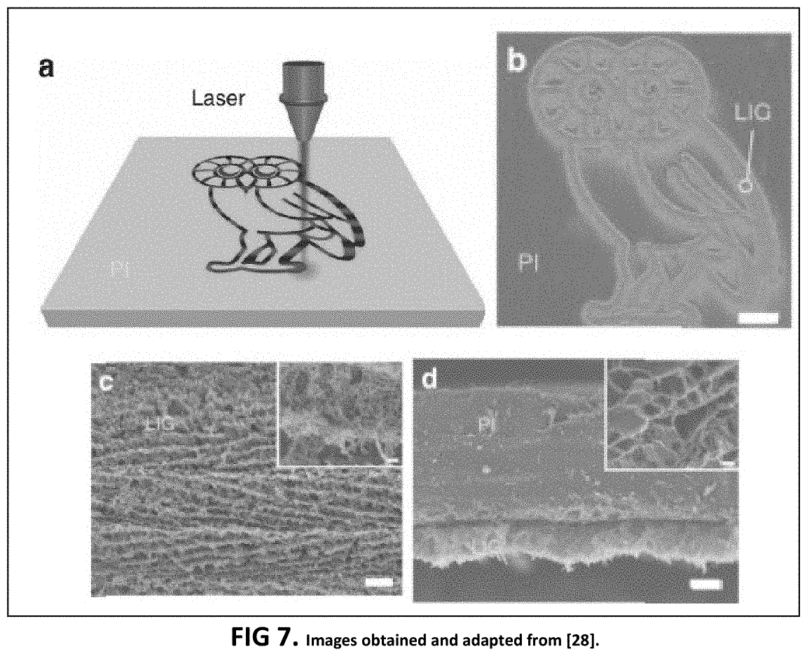

A more complex IDE pattern (see, e.g., FIG. 5) is another (see also—FIG. 7 which shows a still further complex laser scribed pattern from [28] which is possible to achieve with the present invention as another non-limiting example). Background information on IDEs and their operation can be found at U.S. Pat. No. 5,958,791 (M. Roberts, et al.) and U.S. Patent Application Publication US2018/0059101 A1 (S. MacKay et al.), each of which is incorporated by reference herein.

The ability to focus and control in-plane movement of a laser 30 relative a surface 12 with very high resolution (at least μm scale features), provides tremendous versatility in creating patterns, whether one continuous pattern over a given part of a surface or multiple patterns over the same surface area. A subtle aspect of some embodiments of the invention is also control of the laser beam focus point out-of-plane, as will be described further herein.

The designer is provided with the ability to design and create such variety of patterns with available design and laser control systems. Thus, the scale of such LIG electrode-based apparatus according to the invention can vary from quite small (at least μm scale features and working areas) to larger if desired. As such, at the smaller end of scale and form factor, micro-scale individual sensors are possible or sets of micro-scale sensors. They can be easy to emplace for measurement, including in small volume samples. The LIG-pattern is functionalized appropriately for a given electrode application. The LIG-based pattern can take advantage of the well-known benefits of graphene, including physical, electrical, thermal, and other benefits.

Some specific examples focus on pathogen detection by emplacing relevant antibodies in the sensing portion of the LIG pattern. As will be appreciated, a variety of functionalizations are possible. Background information about functionalization with binding agents, including antibodies for immunosensing, can be found at U.S. Patent Application Publication US2019/0330064 A1 (J. Tour, and U.S. Patent Application Publication US2020/00002174 A1 (J. Tour, et al.), each of which is incorporated by reference herein. Other examples relate to other types of biochemical or electrochemical sensing. Others relate to electrode-based functionalities (e.g. water splitting).

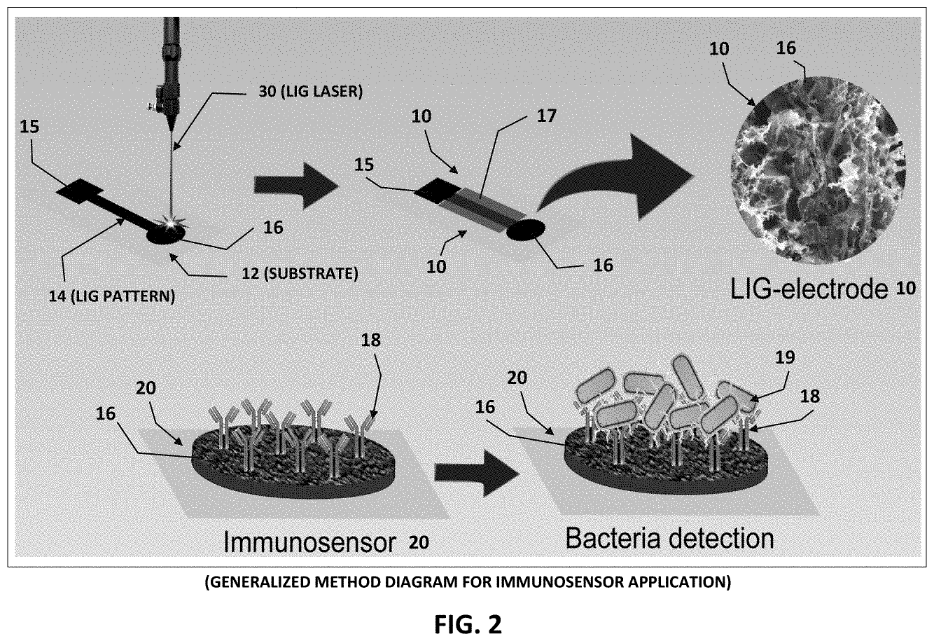

FIG. 2 is a somewhat generalized diagrammatic illustration of a method of creating an apparatus such as in FIG. 1. As indicated above, the method provides substantial design flexibility in what LIG-based pattern 14 might be created. The ability to basically direct-write (e.g. with laser 30) the LIG pattern is a beneficial aspect. And the functionalization can vary according to need or desire.

In this example, a starting suitable substrate 12 which includes a carbon precursor is laser-scribed by spatial and power density control of laser 30 to generate a graphene pattern 14 of desired form factor. In one example, a dip-stick type working electrode pattern is scribed by laser 30, and has one portion 15 adapted for connection to a sensing circuit and the opposite end 16 adapted for functionalization as a sensing surface. In this case, a passivation material 17 can be overlaid pattern 14 between ends 15 and 16 to electrically insulate or block contact with that intermediate portion of pattern 14.

The result is an LIG electrode 10 with an exposed sensing surface or area 16 of porous graphene. Area 16 can be functionalized for a variety of electrode-based purposes, including electrochemical sensing purposes, electrochemical monitoring, and others, for example, water splitting. Examples of how LIG electrodes according to the invention can be functionalized for these varying applications will be discussed in detail infra.

In an example of electrochemical immunosensing illustrated in FIG. 2, antibodies 18 for a target species 19 are immobilized on area 16. This can be by well-known techniques. As such, LIG electrode 10 becomes a biosensor 20 (in this example an immunosensor as the antibodies are those to bind a pathogen/bacteria).

When biofunctionalized end 16 (with immobilized antibodies 18) is exposed to a sample, if the sample contains a species of interest that binds to antibodies 18, by impedimetric techniques, measurements at surface 16 can detect (at ref. no. 30) the presence of the target 19 and, thus, functions as an immunosensor.

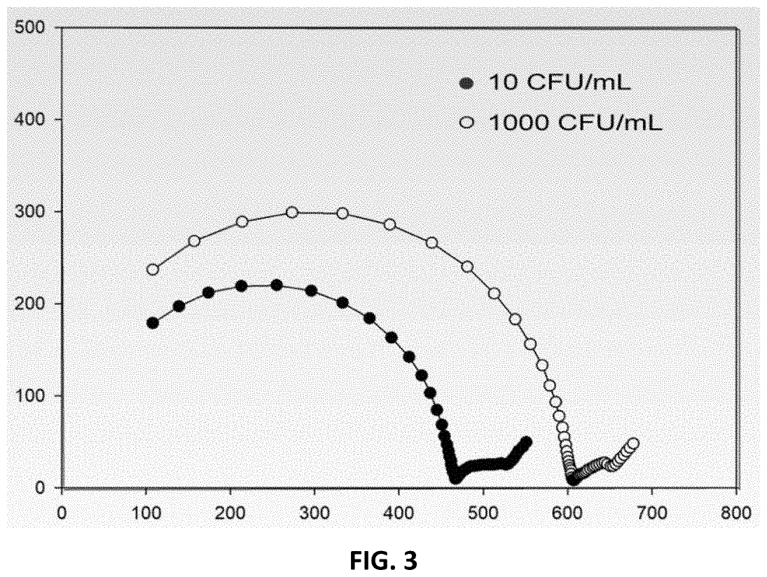

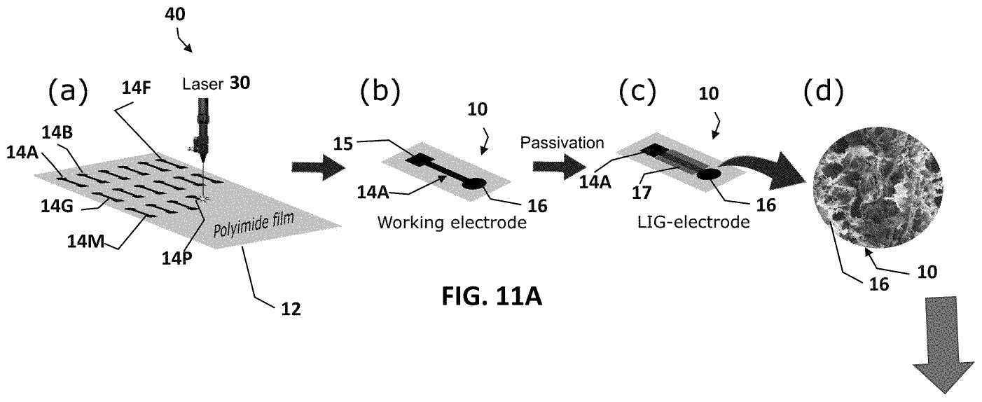

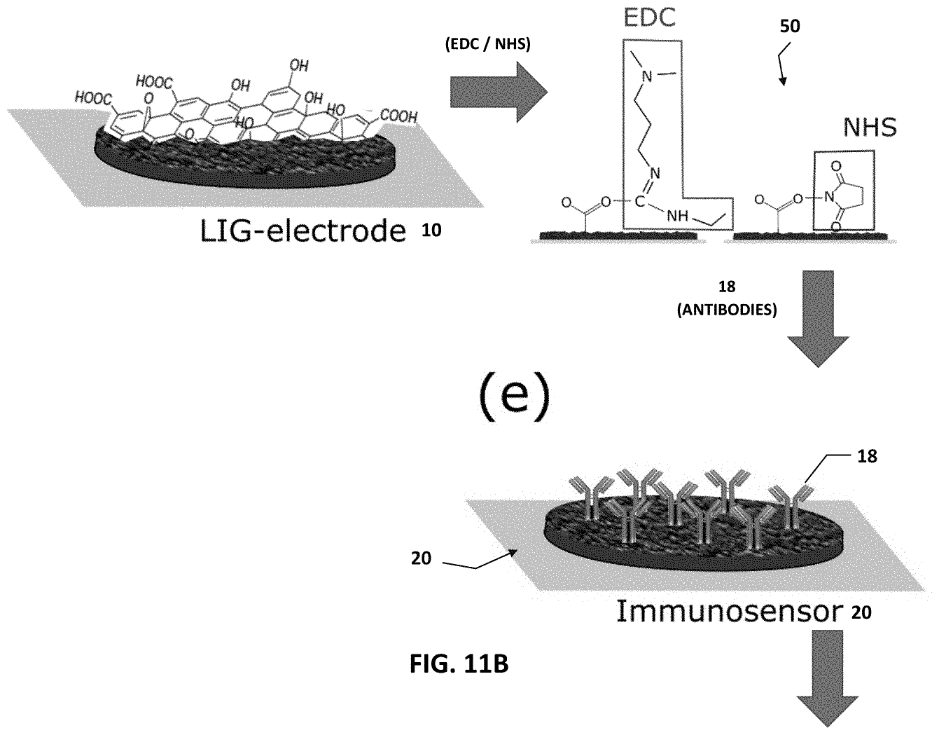

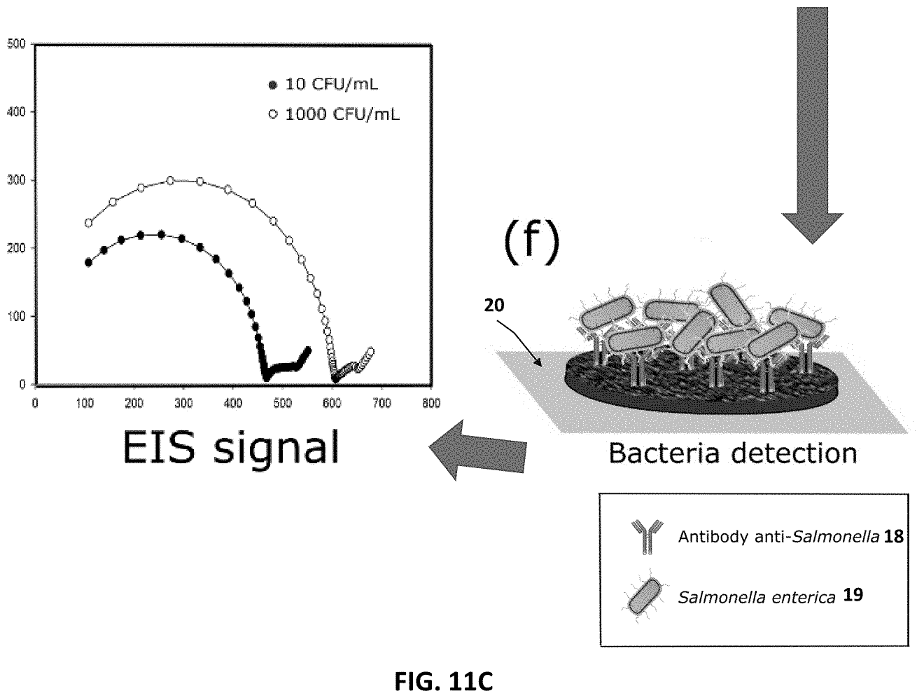

FIG. 3 shows proof-of-concept in relation to testing for bacteria with an embodiment according to the present invention, as discussed in detail with regard to FIG. 11C.

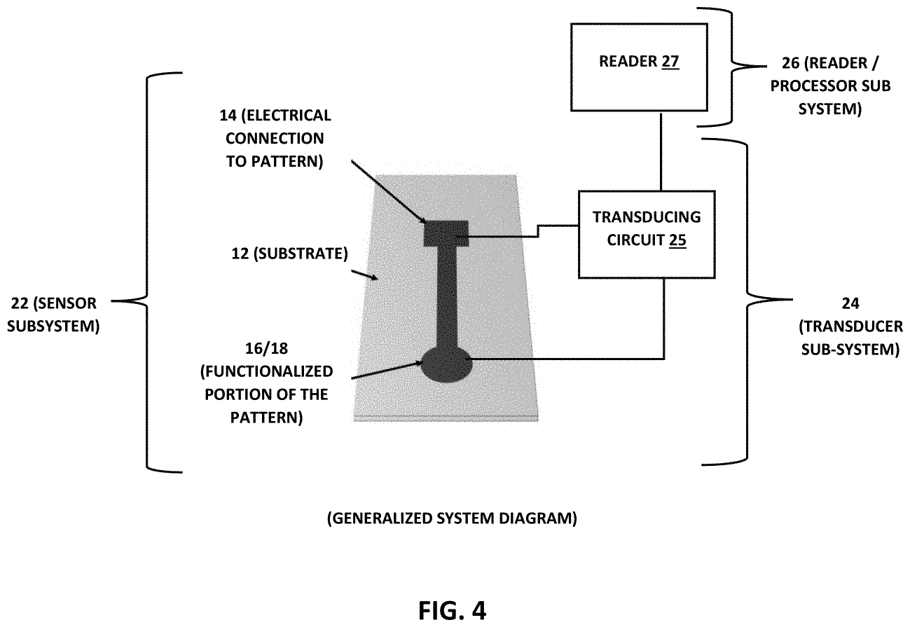

FIG. 4, at a highly diagrammatic level, indicates a system 22 that utilizes an apparatus such as created according to FIGS. 1 and/or 2. The system 22 indicates how an electrochemical sensor system, using the apparatus 10 as an electrode, and bio-functionalized into a bio sensor 20, could be set up. By a transducer subsystem 24 known in the art or otherwise developed according to need or desire, a transducing circuit 25 could be operatively connected to the LIG electrode 10 to conduct impedimetric measurements. An appropriate reader/processor subsystem 26 can include a reader component 27 to read the circuit 25 and quantify electrical signals for purposes of determining the signals indicate the presence of a target species of interest, all as known in the art. Of course, as will be appreciated, different configurations and set-ups will depend on a given application. Other or additional components, appropriate for different application, would be substituted for that shown in FIG. 3. For example, a control circuit and electrical power source would be applied to a set of electrodes functionalized for water-splitting according to one aspect of the invention. Similarly, a control circuit and electrical power would be applied in a manner appropriate for ion-sensing with LIG electrodes according to another aspect of the invention. Similar appropriate configurations for other applications of LIG electrodes according to the invention would be made, as would be within the skill of those skilled in the art.

FIG. 5 illustrates one possible implementation of system 22. Laser system 30 (commercially available) can be programmed to laser-scribe with beam 34 from controllable laser source 30, a laser spot 35 along the carbon-precursor-containing substrate 12, a desired LIG pattern 14. As shown, desired anti-bodies 18 are immobilized on a desired portion of pattern 14. FIG. 5 illustrates that the same LIG electrode 10 could be biofunctionalized with any of a variety of anti-bodies (here two different anti-bodies 18A and 18B are shown, each of which attracts a different pathogen of interest 19A or 19B respectively). Thus, multiple immunosensors 20 could be created to detect different pathogens and be available for a user. FIG. 5 further illustrates that a sensor system 22, including a transducer subsystem 24 and reader/processor subsystem 26 could be contained in a small form factor, even hand-sized or otherwise portable housing. Any of the immunosensors 20A or 20B could be exposed to a sample, and then inserted into device 22. Device 22 would be operated to take impedimetric measurements of immunosensor 20A or 20B and report (via a user interface such as an on-board display or indicator) if a pathogen of interest is indicated to be detected. Optionally, by known techniques, the readings of device 22 can be communicated to other devices (another processor such as a server or computer). This could include be any known communication technique, including but not limited to wireless signaling 29A or blue tooth 29B. As such, systems according to embodiments of the invention can be highly suitable for point-of-use applications or in-field applications. Again, functionalization of the LIG electrodes made according to aspects of the invention would be varied according to a specific application and what would be needed to effectively operate the LIG electrodes for each of those purposes.

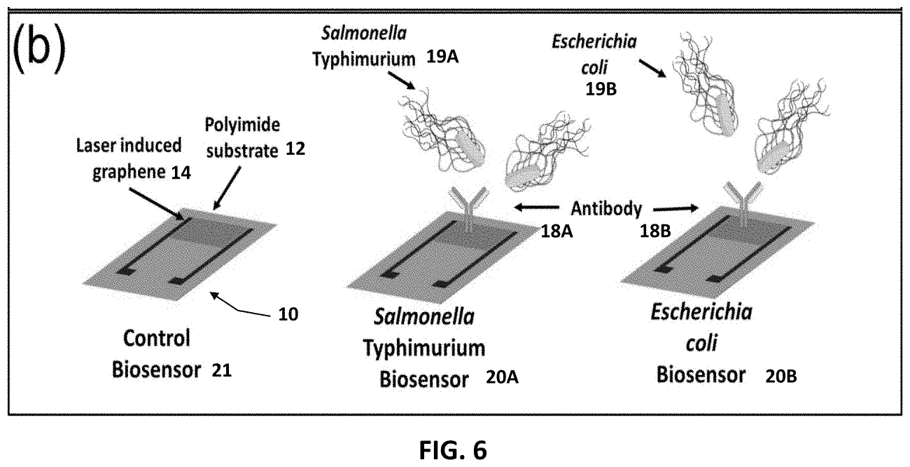

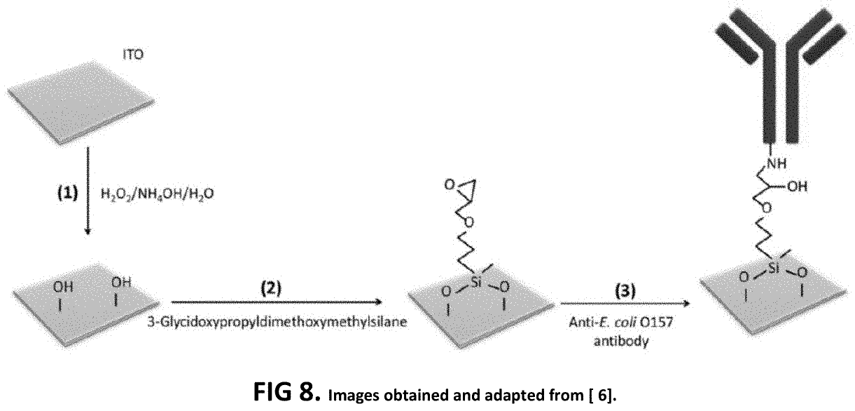

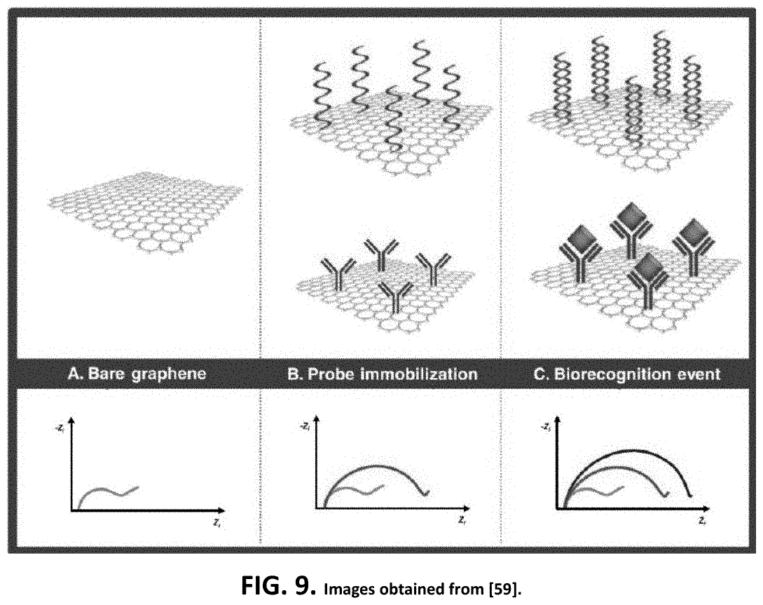

As will be appreciated, techniques to functionalize an electrode sensing area are well-known to those skilled in the art. FIG. 6 shows diagrammatically how two different immunosensors 20A and 20B can be created and available for use. FIGS. 8 and 9 are highly diagrammatic illustrations of how anti-bodies are immobilized, and how electrical measurements can be used for a bio-recognition event at the immobilized anti-bodies (FIG. 8) and application of EIS for biosensing (FIG. 9—images from [59], bottom three curves typical Nyquist Plots). Techniques appropriate for ion-sensing, water splitting, pesticide monitoring or sensing, and other applications of aspects of the invention will be discussed infra.

As such, the apparatus, methods, and systems of the generalized embodiments of FIGS. 1-10 meet at least one of the objects, features, advantages, or aspects of the invention as discussed in this description. An effective, economical, versatile solution to the technical problem of rapid, in-field use can be achieved.

As will be appreciated, the invention can many forms and embodiments. It can also include ancillary or optional features. Some of those are discussed herein.

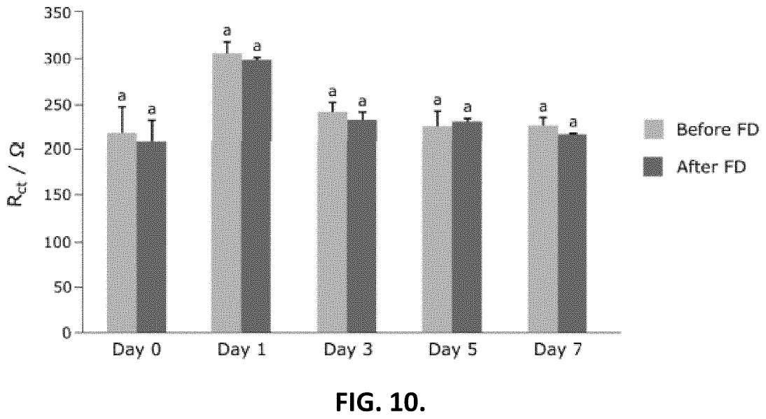

One example applicable to functionalization as an immunosensor is illustrated by FIG. 10. Some anti-bodies will degrade in ambient temperatures. Freeze-drying them can prolong their efficacy. Optionally, immunosensors like 20A and 20B can be pre-prepared and then freeze-dried in ready-to-use form (at −80° C. for 12 h, then freeze-dried for 24 h at −50° C. and 0.130 mbar using a FreeZone 4.5. L Freeze Dryer System (Labconco, Kansa City, Mo., USA, and then sored at −20° C.). A user, thus, has available different immunosensors for immediate use. FIG. 10 is proof-of-concept from experiments showing efficacy of such freeze-drying of immunosensors such as 20A and 20B by showing change in absolute values of charge resistance (R_{CT}/Ω) before and after freeze-drying treatment for each day of evaluation. Impedance measurements were performed at 0, 1, 3, 5, and 7 days of storage. Error bars denote standard error (n=3). Different letters indicate significant difference using t-test at significance level of 5%. This shows immunosensors according to the invention can have a substantial shelf-life. Other options and alternatives relating to immunosensors, or to other applications like other biosensors, electrochemical sensors, monitors, catalysis electrodes, or water-splitting, are discussed infra.

Other example of options, alternatives, and variations possible with aspects of the invention will become apparent herein.

### C. Specific Embodiments and Examples

Specific applications of aspects of the invention, and proof of concept data about them, are now set forth. As will be appreciated, these embodiments and examples meet at least some of the objects, features, advantages, and aspects of the invention.

### 1. Specific Embodiment and Example 1

With particular reference to FIGS. 11A-C to 15A-B, specific embodiments of apparatus, methods, and systems according to the invention are set forth in more detail. This subject, including supporting information cited therein, is publicly available at: Raquel R. A. Soares; Robert G. Hjort; Cicero C. Pola; Kshama Pantie; Efraim L. Reis; Nilda F. F. Soares; Eric S. McLamore, Jonathan C. Claussen, Carmen L. Gomes, Laser-induced graphene electrochemical immunosensors for rapid and label-free monitoring of Salmonella enterica in chicken broth, ACS Sens. 2020, 5, 7, 1900-1911 published Apr. 29, 2020 https://doe.org/10.1021/acssensors.9b02345 copyright 2020 American Chemical Society. Supporting Information is available free of charge at https://pubs.acs.org/doi/10.102/acssensors.9b02345. Both of these are incorporated by reference herein in their entireties.

### Laser-Induced Graphene Electrochemical Immunosensors for Rapid and Label-Free Monitoring of Salmonella enterica in Chicken Broth

### Abstract

Foodborne illnesses are a growing concern for the food industry and consumers, with millions of cases reported every year. Consequently, there is a critical need to develop rapid, sensitive, and inexpensive techniques for pathogen detection in order to mitigate this problem. However, current pathogen detection strategies mainly include time-consuming laboratory methods and highly trained personnel. Electrochemical in-field biosensors offer a rapid, low-cost alternative to laboratory techniques, but the electrodes used in these biosensors require expensive nanomaterials to increase their sensitivity, such as noble metals (e.g., platinum, gold) or carbon nanomaterials (e.g., carbon nanotubes, or graphene). Herein, we report the fabrication of a highly sensitive and label-free laser-induced graphene (LIG) electrode that is subsequently functionalized with antibodies to electrochemically quantify the foodborne pathogen Salmonella enterica serovar Typhimurium. The LIG electrodes were produced by laser induction on polyimide film in ambient conditions, and hence circumvent the need for high-temperature, vacuum environment, and metal seed catalysts commonly associated with graphene-based electrodes fabricated via chemical vapor deposition processes. After functionalization with Salmonella-antibodies, the LIG biosensors were able to detect live Salmonella in chicken broth across a wide linear range (25 to 10⁵ CFU mL⁻¹) and with a low detection limit (13±7 CFU mL⁻¹; n=3, mean±standard deviation). These results were acquired with an average response time of 22 minutes without the need for sample pre-concentration or redox labeling techniques. Moreover, these LIG immunosensors displayed high selectivity as demonstrated by non-significant response to other bacteria strains. These results demonstrate how LIG-based electrodes can be used for electrochemical immunosensing in general and, more specifically, could be used as a viable option for rapid, low-cost pathogen detection in food processing facilities before contaminated foods reach the consumer.

Nearly half a million people die each year from acquiring foodborne illnesses.¹ This dismal statistic is only expected to increase as global food production and trade continue to rise to meet the demands of the increasing world population (over 9 billion by 2050 according to the United Nations prediction²). Hence, efficient food quality control measures are desperately needed to avoid wide-spread foodborne diseases and contamination. Data from Food and Drug Administration (FDA) and Centers for Disease Control and Prevention (CDC) claim that one of the major contributors to foodborne illnesses is the bacteria Salmonella enterica, which causes about 1.2 million illnesses, 23,000 hospitalizations, and 450 deaths in the United States every year.^{3,4} Furthermore, Salmonella causes an estimated $3.7 billion in economic burdens each year with exposure occurring through food, water, and contaminated surfaces.⁵ Despite strict regulations and efforts from producers to control pathogens in the food supply, growing numbers of foodborne illnesses are being reported globally.⁶

The reason for these illnesses is that contaminated food product (whether contaminated in the field or within food processing facilities) still passes unnoticed to the consumer. This is because foodborne pathogen detection is time-consuming and arduous. The gold standards for monitoring these pathogens include bacteria plate counting and polymerase chain reaction (PCR) experiments that may take several days due to pre-enrichment steps and necessary laboratory processing.⁷ Hence, all food products passing through the doors of food processing facilities are not tested for pathogens as some food would spoil before tests could be performed and most food products have low/tight profit margins making ubiquitous testing infeasible.^{8,9} Therefore, there is an urgent need to create a rapid (less than 1 hour), low-cost (less than $1), and highly sensitive (detection limits comparable to plate counting and PCR methods) sensor systems that can be used on-site to detect foodborne pathogens such as Salmonella.^{10,11} Recent research into electrochemical biosensors, including our own,¹²⁻¹⁶ has demonstrated promising potential for such in-field pathogen and containment detection.¹⁷⁻¹⁹

Electrochemical biosensors have been explored extensively in recent years as an alternative to conventional methods for detection of pathogenic bacteria, mainly due to their high sensitivity, easy handling, fast response time, and low costs.²⁰⁻²³ Moreover, comparing them with other commonly used techniques, such as colorimetric and fluorescence assays, electrochemical transducers have significant advantages since they do not require laborious interpretation and equipment resources, exhibit more versatile detection schemes which provide broader applications, and are capable of real-time quantification.^{24,25} Also, electrochemical biosensors have received particular attention since they can perform direct and label-free measurements, and can be easily manipulated by personnel without previous training (e.g., home glucose monitors for diabetics^{26,27}). Moreover, electrochemical biosensors that are modified with carbon nanomaterials such as graphene have significantly improved biosensor performance^{28,29} and have increasingly been applied to food safety and sustainable agriculture applications.^{13,14,30} Recent reports have demonstrated potential in developing sensitive and accurate electrochemical Salmonella detection platforms.^{24,31-33} However, some of these biosensors are complex and costly because they require expensive (noble metals^{34,35}) or difficult to fabricate materials (e.g., gold nanoparticles biofunctionalized with enzymes,³⁶ nanocomposites using graphene oxide and titanium isopropoxide³⁷) to improve signal amplification and/or complex manufacturing steps (e.g., cleanroom processing³⁸). It should also be noted that the main challenge in the field of biosensing for monitoring pathogens is not the response time, but the poor detection limit, approximately 10²-10³ CFU mL⁻¹ (detection limits of ≤5 CFU mL⁻¹ are required for ensuring pathogen-free food products^{10,17}). To overcome this hurdle, most studies have integrated a pre-concentration step, which improves detection limit, but obfuscates the rationale for creating the rapid sensor in the first place. However, a graphene biosensor may help to overcome these detection limit shortcomings by improving the sensor sensitivity.

Within the category of two-dimension materials, graphene is considered outstanding due to its structure and exceptional properties.^{39,40} Graphene is a sheet of sp² bonded carbon atoms arranged into a rigid hexagonal lattice, exhibiting a set of properties that no material has concomitantly displayed; for example, high mechanical strength (10¹² Pa), excellent electrical conductivity and charge carrier mobility (˜10⁵ cm²V⁻¹s⁻¹) large specific surface area (˜2630 m²g⁻¹), and high impermeability and biocompatibility.⁴¹⁻⁴³ Consequently, graphene-like nanomaterials have attracted attention as emerging materials for electrochemical sensor applications.⁴⁴ Techniques for graphene electrode fabrication have grown considerably to supply the demand for this material;³⁹ however, common methods of synthesis, such as photolithography^{45,46} (an expensive cleanroom processing technique), chemical vapor deposition (CVD),⁴⁷ laser ablation⁴⁸ include high thermal requirements, low-pressure (vacuum) requirements, or multiple steps towards chemical formation of graphene.⁴⁹ Moreover, post-synthesis processing is generally required to transfer the graphene to a non-conductive substrate⁵⁰ which further increases the time and cost of electrode fabrication. An alternative to these expensive graphene electrode fabrication techniques is to produce sensors based on direct-write processes such as inkjet printing or aerosol jet printing that are capable of printing graphene electrodes from graphene flakes synthesized from bulk chemical exfoliation of graphite.⁵¹⁻⁵⁴ Though these graphene-electrode fabrication methods do not retain the high-performance characteristics of pristine CVD-grown graphene, for example, they do display sufficient electrical conductivity and biocompatibility needed for a variety of sensor applications and eliminate the high cost of alternative graphene synthesis protocols and graphene transfer methods. However, these printing techniques often require additional post-print processing (e.g., laser,⁵³ thermal,⁵⁵ or photonic annealing⁵⁶) to increase the electrical conductivity of the printed graphene which further complicates their fabrication process.

As an alternative to these techniques, the Tour group⁴⁹ introduced a simple one-step, direct-write graphene electrode fabrication method, called laser-induced graphene (LIG). LIG is typically formed by converting sp³ carbon found in polyimide into highly conductive sp² hybridized carbon found in graphene through CO₂ laser induction^{57,58} (though some have demonstrated LIG formation on polyimide using a UV laser^{59,60}). LIG combines both the graphene synthesis and graphene electrode fabrication steps into one simple process, using a laser to selectively convert distinct patterns of polyimide into a high-surface graphene circuit that is often nano/microstructured or porous. Since LIG can be easily manufactured from commercial polymers, it has been applied towards stretchable and sensitive strain gauges,⁶¹ non-biofouling surfaces,⁶² microsupercapacitors,⁶³ UV photodetectors,⁶⁴ sound generators and detectors,⁶⁵ and more recently, electrochemical sensors.⁶⁶ Recently, an electrochemical biosensor based on LIG-electrodes was developed for the detection of biogenic amities in food samples;⁵⁹ similarly, in another study an electrochemical biosensor was developed based on LIG that showed the ability to detect low levels of the antibiotic chloramphenicol, which is banned in food production.³⁵ Another example is the electrochemical LIG-sensor used for fouling-biofilm detection, one of the main challenges in the food industry,⁶² while another LIG sensor was capable of monitoring the concentration of nitrogen (both ammonium and nitrate ions in soil solutions) in the hopes of better monitoring and controlling fertilizer inputs in farm fields to maximize crop yield while lowering fertilizer waterway pollution due to excess fertilizer use.⁶⁰ However, electrochemical pathogen sensing using LIG has yet to be demonstrated.

Herein, we report on the first LIG sensor that is capable of rapid and quantifiable detection of Salmonella enterica concentrations in food samples. Porous graphene was produced from polyimide by laser induction, and then characterized conferring a new potential application in the sensing field. An impedimetric immunosensor was developed based on LIG-electrodes functionalized with specific antibodies for detection of Salmonella enterica, one of the most prominent foodborne pathogens.⁶⁷ The immunosensor was able to detect the pathogen at low concentration, 13±7 CFU mL⁻¹ in complex media, chicken broth, with a response time of 22 minutes. Electrochemical impedance spectroscopy was used as a label-free detection over a broad range of bacteria concentrations, from 25 to 10⁵ CFU mL⁻¹. Moreover, this promising device is a low-cost and disposable sensor that can be used in-field or at the point-of-service (e.g., food processing facilities) for the detection of contamination, which reinforces its important contribution to food safety.

### Material and Methods

### Materials

Polyimide (Kapton, 0.07 mm) tape was purchased from McMaster-Carr co. (Elmherst, Ill., USA), and Epson Ultra Premium Photo Luster (240 g m⁻²) was acquired from Office Depot (Boca Raton, Fla., USA). Potassium ferro/ferricyanide, N-Hydroxysuccinimide (NHS), 1-Ethyl-3-(3-dimethylaminopropyl) carbodiimide (EDC), 2-(N-morpholino) ethanesulfonic acid (MES), and ethanolamine were purchased from Sigma-Aldrich (St. Louis, Mo., USA). Tryptic soy agar (TSA), tryptic soy broth (TSB), tryptose phosphate broth (TPB), and buffered peptone water (BPW) were purchased from Criterion Dehydrated Culture Media (Hardy Diagnostics, Santa Maria, Calif., USA). Potassium chloride and SuperBlock™ in phosphate buffered saline (PBS) (used as blocking buffer) were purchased from ThermoFisher Scientific (Waltham, Mass., USA). KPL BacTrace polyclonal antibody anti-Salmonella was purchased from SeraCare (USA). PBS was purchased from Alpha Aesar (Tewksbury, Mass., USA), and chicken broth was purchased from a local supermarket. All the chemicals used in this study were analytical grade. Solutions were made using deionized with an electric resistance of approximately 18.2 MΩ.

### Laser Induced Graphene Electrode Fabrication

The working electrode was designed using a linear sketch pattern (0.17 mm separation) in SolidWorks 2018 (Dassault Systems, France), and the engraving process was performed with a 75 W Epilog Fusion M2 CO₂ laser (Epilog Laser, Golden, Colo., USA) at 7% speed and 4% power with a lens to material distance of ˜74 mm and beam size of ˜176 μm in ambient atmosphere. The laser induction was carried out on polyimide film taped onto the emulsion side of the photo paper, as previously described by Tehrani and Bavarian,⁶⁸ and Fenzl et al.⁶⁹ This procedure 40 produced LIG-electrodes as shown in FIGS. 11A-D. The working area 16 (3-mm diameter) and connector 15 ends of the working electrode 10 were separated by a layer of fast drying lacquer (passivation layer 17) used to cover the non-active areas of the electrodes. Passivation was done to maintain a constant area of the working electrode in contact with the redox solution during electrochemical sensing.⁶⁰

### Material Characterization

The Raman spectrum was obtained by using a Renishaw InVia confocal Raman microscope with a 633-nm laser source (0.12 mW), a 50× objective lens and a diffraction grating of 1800 lines, in order to confirm the graphene formation by the laser induction process. The crystallinity of the bare electrodes and the level of graphitization were evaluated using a Bruker D8 DISCOVER X-ray Diffractometer provided with copper radiation (λ=1.542 Å) scanning θ/2θ. A scanning electron microscope (SEM) JEOL JSM-6010LA equipped with an energy dispersive spectroscopy (EDS) system was used to obtain images of the LIG morphology at 230×, 2000×, and 2300× magnification, and the electrode's chemical composition, at accelerating voltage of 5 kV.

Antibody functionalization onto LIG-Electrodes

To determine the optimum concentration of polyclonal antibody anti-Salmonella to functionalize the LIG-electrodes, different concentrations of antibody were initially functionalized on the electrode surface in an effort to maximize immunosensor performance. Briefly, the working area 16 of the electrodes 10 was covered with 30 μL of EDC/NHS (3:1) solubilized in sterile filtered MES (pH 6.0) for 1 hour and then rinsed with 1× dilution of PBS (1×PBS) pH 7.4 to remove the unreacted EDC/NHS. Next, polyclonal antibody 18 anti-Salmonella at different concentrations (0.5, 1.0 and 1.5 μM) was applied to the surface of the working electrode 10 followed by overnight incubation at 4° C. See method 50 at FIGS. 11E-F. The electrode was then rinsed with 1×PBS, dried at room temperature, and afterwards, 1 M ethanolamine was applied for 20 minutes to quench the remaining unreacted EDC/NHS. The unreacted graphitic surface was blocked using Superblock in 1×PBS, for 20 minutes, to reduce non-specific binding, and then rinsed off with 1×PBS, prior to testing.

### Electrochemical Characterization

The electrochemical proprieties of the LIG-electrodes 10 were analyzed using cyclic voltammetry (CV) and electrochemical impedance spectroscopy (EIS). All electrochemical measurements were carried out on a CH Instruments Electrochemical Analyzer (CHI7081E model, CH Instruments, Inc., Austin, Tex., USA) at room temperature. The 3-electrode system consisted of a CH Instruments Ag/AgCl reference electrode, platinum counter electrode, and the LIG as the working electrode 10. CV and EIS experiments were carried out in 10 mL solution containing 0.1 M KCl, 4 mM K₃[Fe(CN)₆], and 4 mM K₄[Fe(CN)₆]. The scan rates used for CV measurements were 50; 75; 100; 125; 150; 175; 200 mV s⁻¹, in a sweep range from −0.4 V to 0.6 V with a quiet time of 2 seconds between sweeps. The average sheet resistance, n=3, was taken at ambient conditions (25° C.) on a Variable Temperature Hall Effect Measurement System (Model H5000, MMR Technologies, San Jose, Calif., USA). EIS analyses were performed in the frequency range of 1 MHz 100 Hz, using AC amplitude of 10 mV and DC voltage of 0 V.

### Bacteria Sample Preparation

Salmonella enterica subsp. enterica serovar Typhimurium (ATCC 14028), Bacillus cereus (ATCC 14579), Escherichia coli O157:H7 (ATCC 43895), Listeria monocytogenes (ATCC 15313), Pseudomonas aeruginosa (ATCC 10145), Staphylococcus aureus (ATCC 29213) were used to test the immunosensor 20. Bacteria strains stored at −80° C. were resuscitated through 2 consecutives 24 h growth cycles in TSB at 35° C. L. monocytogenes was resuscitated under the same time and temperature conditions in TPB. B. cereus was also resuscitated twice in TSB for 24 h but at 30° C. Bacteria cultures were renewed weekly in TSB or TPB (i.e., one transfer followed by 24 h incubation in aerobic conditions) and maintained at 4° C. Samples of bacteria were serially diluted in BPW, plated via spread plating on TSA and incubated for 18 hours at 35° C. or 30° C. before counting the colony growth, and results were reported as CFU mL⁻¹. Different bacteria concentrations, ranging from approximately 25 to 10⁷ CFU mL⁻¹, were prepared in 15 mL of BPW or chicken broth in order to evaluate the impedimetric immunosensor 20 and to simulate its application in food. Plate counting was used parallelly to the immunosensing experiments to confirm the concentration of the bacterial dilutions and validate the impedimetric results.

### Bacteria Sensing and Selectivity Test

The presence of bacteria was evaluated by EIS analysis, measuring different bacteria concentrations directly in suspension with incubation time of 20 minutes under 180 rpm stirring and analysis time of 90 seconds. Before testing the immunosensor in complex media, its performance was verified in pristine buffer, BPW. Between each measurement, the electrode was thoroughly washed with ix PBS to remove unbound bacteria. Complex plane diagrams (Nyquist plots) were used to determine the charge transfer resistance (R_{ct}), the solution resistance (Rₛ), the double-layer capacitance (C_{dl}), and the Warburg element³⁷ (Z_{w}), fitting the EIS data sets to an equivalent circuit model (i.e., Randles-Ershler circuit) through EIS Spectrum Analyser from ABC Chemistry (Minsk, Belarus). It should be noted that the diameter of the semicircle obtained from Nyquist plots is a measure of the charge transfer resistance (Rd) used to calibrate the concentration of Salmonella attached to the developed biosensor as explained in greater detail in the Results and Discussion section. The LIG-based immunosensor 20 was also evaluated through a selectivity test using the following five foodborne pathogens 19: Escherichia coli, Pseudomonas aeruginosa, Bacillus cereus, Staphylococcus aureus, and Listeria monocytogenes. These bacteria were chosen due to their importance to food safety and were tested under the same conditions used for Salmonella enterica at a constant concentration of 10⁴ CFU mL⁻¹.

### Data Analysis

The measurements were made in triplicate and results were expressed as mean=1, standard deviation. Differences between variables were tested for significance using one-way analysis of variance (ANOVA) and significantly different means (p<0.05) were designated using Tukey's Honestly Significant Differences (HSD) test through IMP v.13 Software (SAS Institute, Cary, N.C., USA). The functional correspondence among quantitative variables was performed using SigmaPlot 12 (Systat, San Jose, Calif., USA) by regression analysis. To evaluate the electroactive surface area (ESA) and the heterogeneous electron transfer rate (HET), the peak current values and peak potential separation from the CV results were used to solve the Randles-Sevick equation^{59,66} and to apply the Nicholson method for reversible electron transfers,⁷⁰ respectively. The 3G method was used to calculate the limit of detection (LOD) and sensivity.^{59,71} Please, see Supporting Information for further details on calculations and data presentation and analysis.

### Results and Discussion

### LIG-Electrodes Characterization

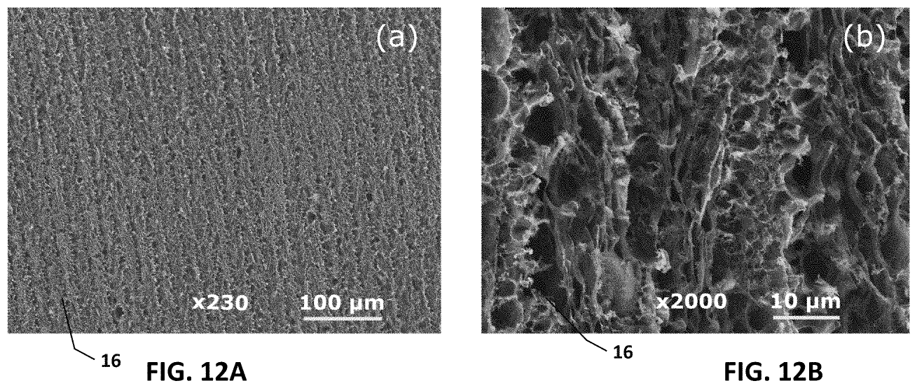

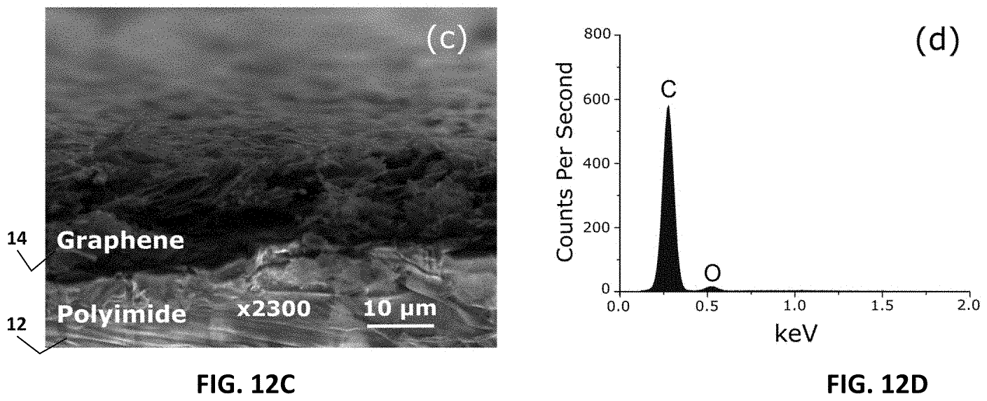

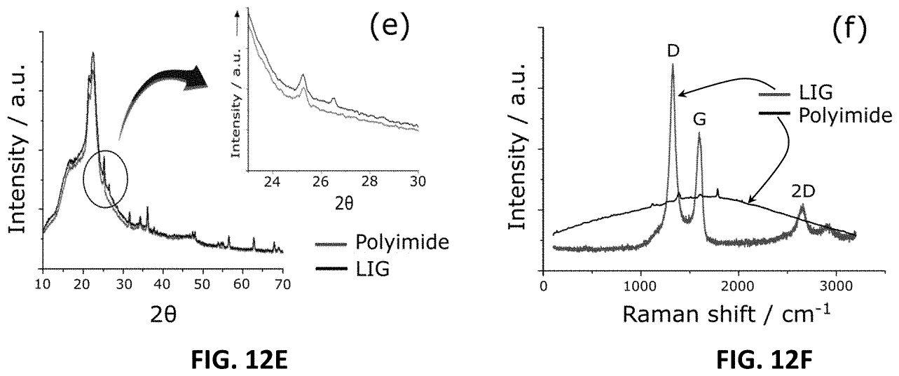

First, SEM was used to characterize the surface topography of the LIG electrodes 10 (FIGS. 12A-C). A carbon structure in hexagonal-planar configuration was formed, as well as a highly porous 3D electrode rich in edge-planes pyrolytic graphite (EPPG). The cross-sectional image (FIG. 12C) shows the LIG-electrode 10 as a macroporous/mesoporous structure with a thickness of 15-20 μm. The irradiation from this laser produced porous graphene onto polyimide film by converting the carbon from polyimide into graphitic carbon.⁴⁹ More specifically, the lasing process converts the sp³ carbon into sp² by photothermal effects, due to the high temperatures reached at the surface (>1000° C.).^{43,49} As demonstrated in FIGS. 12A-C, this ablation procedure is able to provide a carbon frame organized into long-range ordered graphene layers.⁷² According to Nayak et al.,⁶⁶ the available edge-plane sites formed on the surface of the LIG-electrodes 10 contribute to the electron transfer. The 3D morphology confers a higher and more accessible electrochemical surface area, allowing electrolyte penetration more easily into the active area 16.

Next, EDS, Raman spectroscopy, and X-Ray Diffraction (XRD) were performed to analyze the structure of the materials, as well as the surface molecular groups on the LIG electrode 10. The C—O, C—N and C═O bonds originally present in polyimide film 12 could easily be broken by the high temperature,⁴⁹ as confirmed by EDS (FIG. 12D). Assuming (C₂₂O₅N₂H₁₀)ₙ as the polymer chain present in Kapton tape,⁷³ the initial composition could be calculated to be 69.1%, 21.0%, 7.3% and 2.6% m/m for C, O, N and H, respectively, which was converted to 97.5% C and 2.5% O after the lasing process (FIG. 12D), with N, H and O being released as gases due to the high localized heating.⁴³

Raman spectroscopy was used to determine the graphitic properties of LIG. This technique is also useful to characterize disorder in the resultant sp² carbon lattice.⁴³ The Raman spectrum showed three main peaks displayed in FIG. 12F. The first order D peak (roughly at 1350 cm⁻¹) indicates lattice defects caused by bends or breaks in the sigma bonds; the first order G peak (roughly at 1580 cm⁻¹) shows the lattice vibrations of the sp² carbon atoms; while the second order 2D peak (roughly at 2660 cm⁻¹) shows a distinctive peak of graphene structure.^{68,74} The ratio I_{2D}/I_{G} refers to the number of graphene layers, and according to the obtained ratio I_{2D}/I_{G}˜0.35 multilayer graphene was formed.^{75,76} As expected, these peaks were not observed on original polyimide film (FIG. 12F). A complementary analysis of LIG-electrodes by XRD, displayed a peak located at 2θ=26.5° (FIG. 12E). A very similar result was reported by Nayak et al.⁶⁶ at 2θ=26.4°, and also a peak at 2θ=25.9° was reported by Chen et al.,⁵⁸ Lin et al.,⁴⁹ and Zhang et al.,⁷² indicating the presence of C (002) peak, with an interlayer spacing CFO of ˜3.36 Å between LIG-planes, which indicates a high degree of graphitization.⁴⁹

### Electrochemical Characterization

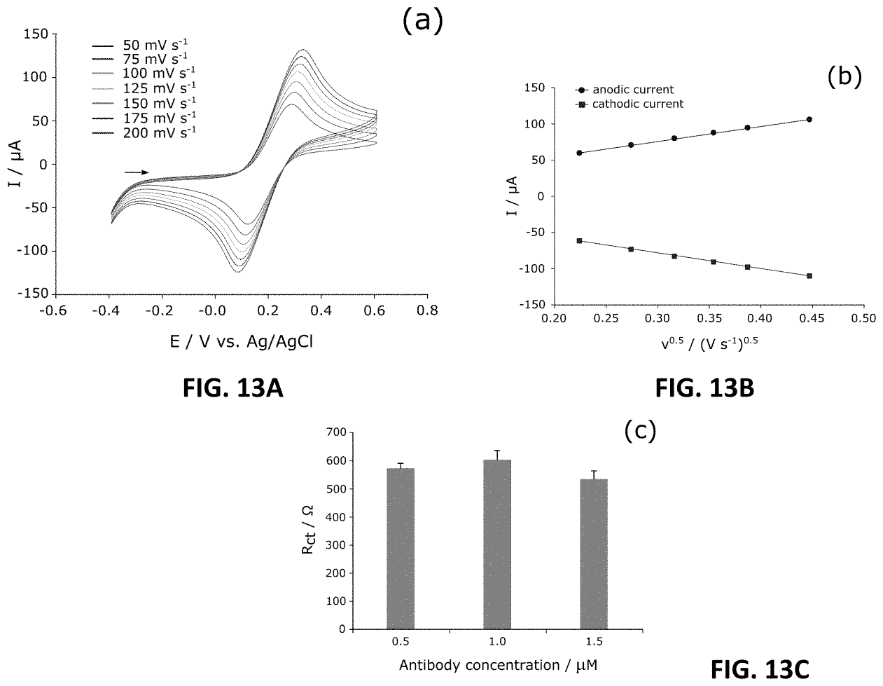

The electrochemical performance of the bare LIG-electrodes 10 was investigated in order to verify its ability to act as an electrochemical transducer. CV curves were recorded and for all scan rates tested the electrodes displayed well-defined redox peaks (FIGS. 13A-B), disclosing its quasi-reversible behavior.⁷⁰ The change in peak separation (ΔEₚ=166 mV-245 mV) observed from these curves indicated a slower electron transfer rate compared to a reversible system (ΔEₚ=60 mV), which is derived from the presence of defects on the EPPG,⁶⁶ previously shown by the Raman spectrum (FIG. 12F). The electroactive surface area (ESA=0.104±0.032 cm²) was approximately 50% higher than the geometric area (0.071 cm²), similar to Nayak et al.⁶⁶ findings, who reported ESA=0.092±0.015 cm² for the same geometric area. This is likely due to the porous graphene structure that increases the surface area which exposes more edge planes of graphene to the redox solution, helping the electron transfer and, therefore, increases the ESA.^{69,77}

The CV curves also convey information about the heterogeneous electron transfer rate (HET) between the electrode 10 and the redox mediator species.⁶⁶ The HET constant obtained (k⁰=0.0146±0.0031 cm s⁻¹) exceeds those found by other groups^{59,69,78} ranging from 0.0030 to 0.0044 cm s⁻¹ for LIG with the same redox ferro/ferricyanide species. It also exceeds commercial edge plane pyrolytic graphite (0.0026 cm s⁻¹) and basal plane pyrolytic graphite (0.0003 cm s⁻¹), as reported in a previous study by Griffiths et al.⁷⁷ These results confirm the effective electron transfer kinetics of LIG produced in this study, and its subsequent feasibility for use as an electrochemical transducer. Furthermore, the average sheet resistance of the LIG-electrodes 10 was 12.7±1.6 kΩ sq⁻¹, which is significantly lower than previously reported values of LIG based electrodes (15-20 kΩ sq⁻¹),⁶⁰ and also lower than electrodes based on inkjet-printed graphene with reported sheet resistance of 34 kΩ sq⁻¹.⁵² Thus, the results obtained from CV, EIS and sheet resistance confirm that the LIG-electrode 10 fabricated in this study is suitable for electrochemical sensing.

### Immunosensor Performance

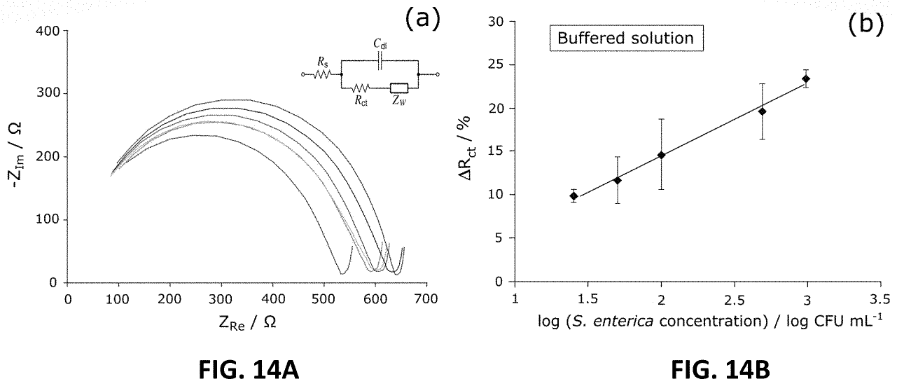

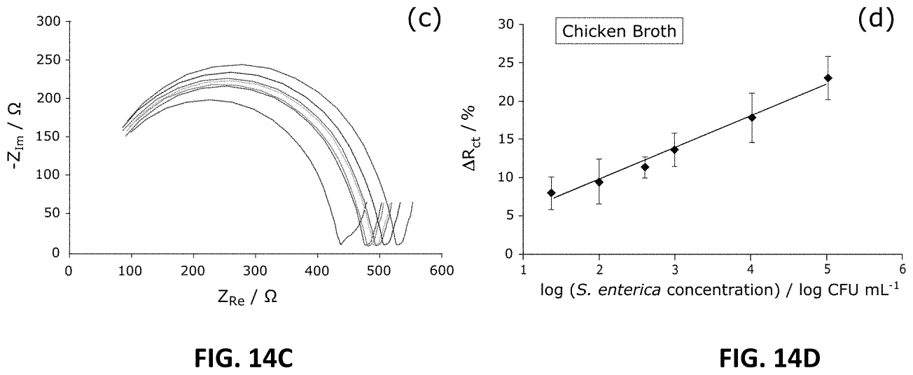

The bare LIG-electrode 10 was converted into an immunosensor 20 by functionalizing the surface with polyclonal antibodies 18 to detect Salmonella enterica Typhimurium 19 via carbodiimide cross-linking (see methods), as shown in FIGS. 11A-C. After the functionalization, the R_{ct} values of these electrodes were calculated in order to assess whether changing the antibody concentration would influence its immobilization on the electrode surface. Results showed no significant difference (p>0.05) among antibody loading concentrations (0.5, 1.0 and 1.5 μM), with ΔR_{ct} ranging around 1-2% (FIG. 13C). Therefore, 1.0 μM was chosen since it has already been shown in previous studies to obtain a good sensing range.⁷⁹ Salmonella enterica detection was evaluated with EIS, and the change in R_{ct} was used to produce the calibration curve in both BPW and chicken broth. Change in the R_{ct} is proportional to the adhesion of bacterial cells to the biofunctionalized region of the electrode.²² This “bio-barrier” hinders the electrolyte access, acting as an electron blocker, therefore increasing the R_{ct}.^{20,80,81} According to this technique a larger diameter corresponds to a larger R_{ct}, which represents a greater number of bacteria binding to the antibodies on the surface of the electrode.²² FIGS. 14A-D displays the Nyquist plot, a typical impedance spectrum, which shows the increase in R_{ct} with increasing Salmonella enterica concentration, obtained from testing the immunosensor 20 in both suspensions, BPW and chicken broth (FIGS. 14A and 14C, respectively). A linear increase in the % ΔR_{ct} as a function of bacteria concentration is also shown for BPW and chicken broth (FIGS. 14B and 14C, respectively).

The presence of attached bacteria cells 19 plays the role of electron kinetic barrier as well as steric hinderance,²⁰ decreasing the electron transfer path between the electrolyte solution and the electrode, and consequently resulting in the increase of R_{ct} values. A calibration plot was obtained by normalizing the R_{ct} with respect to the R_{ct} value measured for zero concentration of Salmonella enterica in the buffer solution. The LIG-based immunosensor 20 presented a linear sensing range from 25 to 10³ CFU mL⁻¹ (R²=0.984), with sensitivity of 42Ω log CFU⁻¹ mL and a limit of detection of 10 CFU mL⁻¹ in buffer (FIGS. 14A-B). To demonstrate the potential of the LIG-based immunosensor 20 in the evaluation of real food samples, chicken broth was used as the sensing matrix. Similarly, a calibration plot was obtained by normalizing R_{ct} values with plain chicken broth. Based on the calibration plot, the linear sensing range for Salmonella enterica detection in chicken broth was between 25 and 10⁵ (R²=0.989) with a sensitivity of 24Ω log CFU⁻¹ mL and a limit of detection of 13 CFU mL⁻¹ (FIGS. 14C-D). The total response time for all immunosensing tests was 21.5 minutes, which consisted of 20 min to allow bacteria contact with the LIG electrode (incubation) and 90 s to collect EIS measurements.

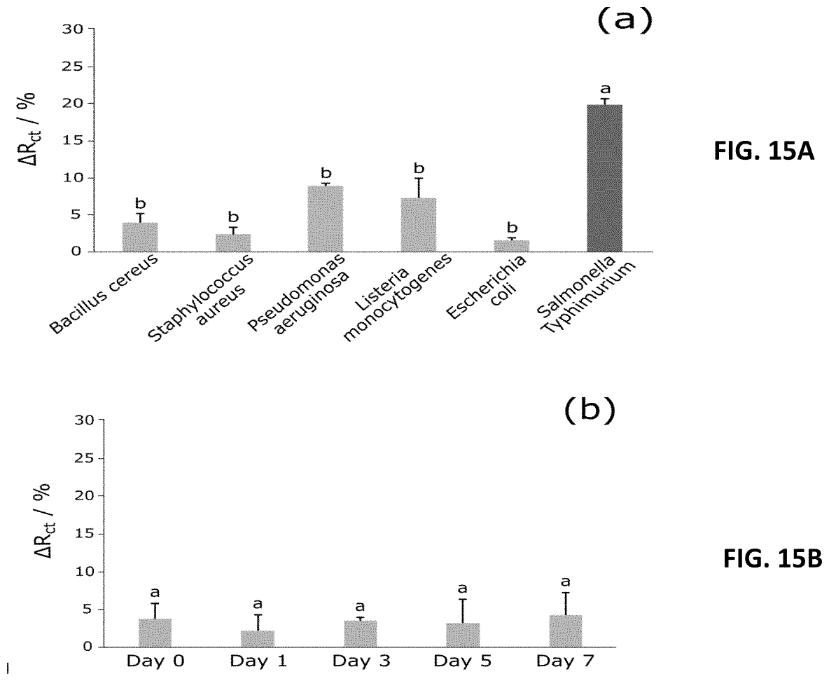

Further, the LIG-based immunosensor was tested for selectivity using 5 different bacteria strains under the same conditions as those used for Salmonella enterica in chicken broth at 10⁴ CFU mL⁻¹. The R_{ct} values recorded from interference testing did not show significant change among the bacteria tested and presented an average value of 4.8% for the ΔR_{ct} (FIG. 15A). Meanwhile, the average ΔR_{ct} value for Salmonella enterica was 4× higher (19.8%, p<0.05) emphasizing the specificity of the developed immunosensor to the targeted pathogen (Salmonella enterica Typhimurium), and avoiding any false positive signal due to other strains of bacteria that could possibly be non-pathogenic in nature.

The shelf life of freeze-dried immunosensors was evaluated during 7 days of storage at −20° C. (see Supporting information for details). As it can be observed in FIG. 15A no difference (p>0.05) was observed in the relative R_{ct} (%) values of the freeze-dried immunosensors. The averaged change in R_{ct} was 3.36% which demonstrated the stability of the developed immunosensors for at least 7 days. Similarly, absolute R_{ct} (Ω) values before and after the freeze-drying process for each day of analysis did not change significantly (see FIG. 10). The freeze-drying technique allows the storage of the immunosensor for extended periods of time, which is advantageous for point-of-service applications and crucial for commercialization.

The developed immunosensor exhibited overall good performance using easily obtainable and inexpensive materials with an estimated materials cost of $1.76 per immunosensor (approximate cost breakdown: polyimide=$0.15, EDC-NHS=$0.01, Superblock=$0.03, ethanolamine=$0.04, antibodies=$1.53), which contributes to its accessible fabrication. Table 1 (below) summarizes the performance characteristics of the immunosensor prepared in this work, as well as other similar biosensors in the recent literature. Previous studies have developed highly sensitive and label-free Salmonella spp. sensors, for example sensors reported by Silva et al.⁶⁷ and Punbusayakul et al.⁸² based on ion selective electrodes made of gold nanoparticles and double-walled carbon nanotubes, respectively, but all require an hour to multiple hours to obtain a signal which is longer than 22 minutes, the response time reported herein. Moreover, biosensors that displayed performance similar to this work used expensive materials and/or complex fabrication methods, such as gold^{67,83} or required multi-step fabrication to develop the electrodes.⁸⁴ Furthermore, the sensitivity reached by the immunosensor developed herein was significantly higher than other recent graphene-based sensors, even in complex matrices, with a limit of detection 2× lower than the one obtained by Jia et al.⁸⁴ and 7× lower than the one obtained by Fei et al.⁸⁵ Thiha et al.⁸⁶ and Appaturi et al.⁸⁷ report impressive analysis times (10 minutes and 5 minutes, respectively). However, both report the necessity of various pieces of laboratory equipment and chemicals leading to a much more complex and longer fabrication process than reported in this work. Since this work reports a process that requires only a Laser and a polyimide substrate for electrode fabrication, it has the advantage of easier upscaling for mass production. These devices also use sample volumes of 5 μL and 1 mL, respectively, which might require sample preconcentration steps to avoid false negatives and consequently increase test response time. Based on the performance characteristics shown in Table 1 (below), there are no concomitant records of a rapid (22 minutes or less), label-free, sensitive, and simple to fabricate sensor similar to the one demonstrated in this work that can selectively detect Salmonella enterica from 25 to 10⁵ CFU mL⁻¹, which covers relevant levels for food safety analysis.

### Conclusions

This work reports on a highly sensitive, selective, and easily fabricated impedimetric immunosensor by direct formation of graphene on commercial polyimide film through a laser induction technique. The results obtained reinforce that this sensor can be widely implemented due to its simple fabrication protocols with equipment that is accessible throughout the world. This immunosensor is a versatile device that could be distinctly functionalized for monitoring other pathogens besides Salmonella enterica Typhimurium, depending on the selectivity of the biorecognition agent. The working electrode based on LIG displayed a high ESA and HET with values of 0.104 cm² and 0.0146 cm s⁻¹, respectively, and was functionalized with antibodies for Salmonella enterica detection. The immunosensor presented a limit of detection to the target bacteria of 13±7 CFU mL⁻¹ in complex media, chicken broth, in just 22 minutes without any pre-treatment. In addition, the sensor exhibited a wide linear sensing range, from 25 to 10⁵ CFU mL⁻¹. Therefore, impedimetric immunosensors based on LIG are very promising for bacteria sensing, since it is easily manufactured in ambient conditions compared to other complex fabrication procedures that require CVD⁷⁶ and/or sophisticated substrate-transfer techniques, ink and ink-preparation⁵² or post-printing processes.⁵³ Consequently, resulting in a low-cost fabrication process that produces porous graphene with high electrical conductivity and chemical stability.⁸⁸ All of these properties demonstrate that the developed biosensor is well-suited for use in food safety monitoring and, in general, a platform that could be modified with different biorecognition agents for future electrochemical biosensors.

### Associated Content

### Supporting Information

Supporting information is at https://pubs.acs.org/doi/10.1021/acssensors.9b02345.

### References [See Superscripts Detailed Description of Exemplary Embodiments of the Invention, Supra]

(1) WHO. Executive Summary of Food Borne Diseases—2015. Exec. Summ. Food Borne Dis. 2015, 1-2.
(2) UN/DESA. World Population Prospects 2017 Data Booklet (ST/ESA/SER.A/401). United Nations, Dep. Econ. Soc. Aff. Popul. Div. 2017, 1-24.
(3) FDA. The Reportable Food Registry: A Five Year Overview of Targeting Inspection Resources and Identifying Patterns of Adulteration. U.S. Food Drug Adm. 2016.
(4) Centers for Disease Control and Prevention (CDC). An Atlas of Salmonella in the United States, 1968-2011; Atlanta, 2013.
(5) Hoffmann, S.; Maculloch, B.; Batz, M. B. Economic Burden of Major Foodborne Illnesses Acquired in the United States. Econ. Inf. Bull. 2015, No. 140, 59.
(6) Law, J. W.; Mutalib, N. A.; Chan, K.; Lee, L. Rapid Methods for the Detection of Foodborne Bacterial Pathogens: Principles, Applications, Advantages and Limitations. 2015, 5, 1-19. https://doi.org/10.3389/fmicb.2014.00770.
(7) Clais, S.; Boulet, G.; Van kerckhoven, M.; Lanckacker, E.; Delputte, P.; Maes, L.; Cos, P. Comparison of Viable Plate Count, Turbidity Measurement and Real-Time PCR for Quantification of Porphyromonas gingivalis. Lett. Appl. Microbiol. 2015, 60 (1), 79-84. https://doi.org/10.1111/lam.12341.
(8) Wengle, S. When Experimentalist Governance Meets Science-Based Regulations; the Case of Food Safety Regulations. Regul. Gov. 2016, 10 (3), 262-283. https://doi.org/10.1111/rego.12067.
(9) Cho, I. H.; Ku, S. Current Technical Approaches for the Early Detection of Foodborne Pathogens: Challenges and Opportunities. Int. J. Mol. Sci. 2017, 18 (10). https://doi.org/10.3390/ijms18102078.
(10) Vanegas, D.; Claussen, J.; McLamore, E.; Gomes, C. Microbial Pathogen Detection Strategies. Encycl. Agric. Food, Biol. Eng. Second Ed. 2010, 1-4. https://doi.org/10.1081/e-eafe2-120051868.
(11) Bobrinetskiy, I. I.; Knezevic, N. Z. Graphene-Based Biosensors for on-Site Detection of Contaminants in Food. Anal. Methods 2018, 10 (42), 5061-5070. https://doi.org/10.1039/c8ay01913d.
(12) Sidhu, R.; Rong, Y.; Vanegas, D. C.; Claussen, J.; McLamore, E. S.; Gomes, C. Impedance Biosensor for the Rapid Detection of Listeria spp. Based on Aptamer Functionalized Pt-Interdigitated Microelectrodes Array. Smart Biomed. Physiol. Sens. Technol. XIII 2016, 9863, 98630F. https://doi.org/10.1117/12.2223443. [incorporated by reference herein].
(13) Hondred, J. A.; Breger, J. C.; Alves, N. J.; Trammell, S. A.; Walper, S. A.; Medintz, I. L.; Claussen, J. C. Printed Graphene Electrochemical Biosensors Fabricated by Inkjet Maskless Lithography for Rapid and Sensitive Detection of Organophosphates. ACS Appl. Mater. Interfaces 2018, 10 (13), 11125-11134. [incorporated by reference herein]. https://cloi.org/10.1021/acsami.7b19763.
(14) Hondred, J. A.; Medintz, I. L.; Claussen, J. C. Enhanced Electrochemical Biosensor and Supercapacitor with 3D Porous Architectured Graphene via. Salt Impregnated Inkjet Maskless Lithography. Nanoscale Horizons 2019, 4 (3), 735-746. https://doi.org/10.1039/c8nh00377g. [incorporated by reference herein].
(15) Hills, K. D.; Oliveira, D. A.; Cavallaro, N. D.; Gomes, C. L.; McLamore, E. S. Actuation of Chitosan-Aptamer Nanobrush Borders for Pathogen Sensing. Analyst 2018, 143 (7), 1650-1661. https://doi.org/10.1039/c7an02039b. [incorporated by reference herein].
(16) Rong, Y.; Padron, A. V.; Hagerty, K. J.; Nelson, N.; Chi, S.; Keyhani, N. O.; Katz, J.; Datta, S. P. A.; Gomes, C.; McLamore, E. S. Post Hoc Support Vector Machine Learning for Impedimetric Biosensors Based on Weak Protein-Ligand Interactions. Analyst 2018, 143 (9), 2066-2075. https://doi.org/10.1039/c8an00065d. [incorporated by reference herein].
(17) Vanegas, D. C.; Gomes, C. L.; Cavallaro, N. D.; Giraldo-Escobar, D.; McLamore, E. S. Emerging Biorecognition and Transduction Schemes for Rapid Detection of Pathogenic Bacteria in Food. Compr. Rev. Food Sci. Food Saf. 2017, 16 (6), 1188-1205. https://doi.org/10.1111/1541-4337.12294. [incorporated by reference herein].
(18) Arora, P.; Sindhu, A.; Dilbaghi, N.; Chaudhury, A. Biosensors as Innovative Tools for the Detection of Food Borne Pathogens. Biosens. Bioelectron. 2011, 28 (1), 1-12. https://doi.org/10.1016/j.bios.2011.06.002. [incorporated by reference herein].
(19) Lazcka., O.; Campo, F. J. Del; Munoz, F. X. Pathogen Detection: A Perspective of Traditional Methods and Biosensors. Biosens. Bioelectron. 2007, 22 (7), 1205-1217. https://doi.org/10.1016/j.bios.2006.06.036. [incorporated by reference herein].
(20) Mutreja, R.; jariyal, M.; Pathania, P.; Sharma, A.; Sahoo, D. K.; Suri, C. R. Novel Surface Antigen Based Impedimetric Immunosensor for Detection of Salmonella typhimurium in Water and Juice Samples. Biosens. Bioelectron. 2016, 85, 707-713. https://doi.org/10.1016/j.bios.2016.05.079.
(21) Sheikhzadeh, E.; Chamsaz, M.; Turner, A. P. F.; Jager, E. W. H.; Beni, V. Label-Free Impedimetric Biosensor for Salmonella Typhimurium Detection Based on Poly [Pyrrole-Co-3-Carboxyl-Pyrrole] Copolymer Supported Aptamer. Biosens. Bioelectron. 2016, 80, 194-200. https://doi.org/10.1016/j.bios.2016.01.057.
(22) Barreiros dos Santos, M.; Azevedo, S.; Agusil, J. P.; Prieto-Simón, B.; Sporer, C.; Torrents, E.; Juárez, A.; Teixeira, V.; Samitier, J. Label-Free ITO-Based Immunosensor for the Detection of Very Low Concentrations of Pathogenic Bacteria. Bioelectrochemisiry 2015, 101, 146-152. https://doi.org/10.1016/j.bioelechem.2014.09.002.
(23) Xu, M.; Wang, R.; Li, Y. Rapid Detection of Escherichia coli O157:H7 and Salmonella Typhimurium in Foods Using an Electrochemical Immunosensor Based on Screen-Printed. Interdigitated Microelectrode and Immunomagnetic Separation. Talanta 2016, 148, 200-208. https://doi.org/10.1016/j.talanta.2015.10.082.
(24) Silva, N. F. D.; Magalhaes, J. M. C. S.; Freire, C.; Delerue-Matos, C. Electrochemical Biosensors for Salmonella: State of the Art and Challenges in Food Safety Assessment. Biosens. Bioelectron. 2018, 99 (August), 667-682. https://doi.org/10.1016j.bios.201.7.08.019.
(25) Cinti, S.; Volpe, G.; Piermarini, S.; Delibato, E.; Palleschi, G. Electrochemical Biosensors for Rapid Detection of Foodborne Salmonella: A Critical Overview. Sensors (Switzerland) 2017, 17 (8), 1-22. https://doi.org/10.3390/s17081910.
(26) Wang, J. Electrochemical Glucose Biosensors. Chem. Rev. 2008, 108 (2), 814-825. https://doi.org/10.1021/cr068123a.
(27) Abellán-Llobregat, A.; Jeerapan, I.; Bandodkar, A.; Vidal, L.; Canals, A.; Wang, J.; Morallón, E. A Stretchable and Screen-Printed Electrochemical Sensor for Glucose Determination in Human Perspiration. Biosens. Bioelectron. 2017, 91, 885-89L https://doi.org/10.1016/j.bios.2017.01.058.
(28) Lawal, A. T. Progress in Utilisation of Graphene for Electrochemical Biosensors. Biosens. Bioelectron. 2018, 106 (January), 149-178. https://doi.org/10.1016/j.bios.2018.01.030.
(29) Pumera, M. Graphene in Biosensing. Mater. Today 2011, 14 (7-8), 308-315. https://doi.org/10.1016/S1369-7021(11)70160-2.
(30) Rotariu, L.; Lagarde, F.; jaffrezic-Renault, N.; Bala, C. Electrochemical Biosensors for Fast Detection of Food Contaminants—Trends and Perspective. TrAC—Trends Anal. Chem. 2016, 79, 80-87. https://doi.org/10.1016/j.trac.2015.12.017.
(31) Salam, F.; Tothill, I. E. Detection of Salmonella typhimurium Using an Electrochemical Immunosensor. Biosens. Bioelectron. 2009, 24 (8), 2630-2636. https://doi.org/10.1016/j.bios.2009.01.025.
(32) Niyomdecha, S.; Limbut, W.; Numnuam, A.; Kanatharana, P.; Charlermroj, R.; Karoonuthaisiri, N.; Thavarungkul, P. Phage-Based Capacitive Biosensor for Salmonella Detection. Talania 2018, 188 (April), 658-664. https://doi.org/10.1016/j.talanta.2018.06.033.
(33) Liu, J.; Jasim, I.; Shen, Z.; Zhao, L.; Dweik, M.; Zhang, S.; Almasri, M. A Microfluidic Based Biosensor for Rapid Detection of Salmonella in Food Products. PLoS One 2019, 14 (5), 1-18. https://doi.org/10.1371/journal.pone.0216873.
(34) Pagliarini, V.; Neagu, D.; Scognamiglio, V.; Pascale, S.; Scordo, G.; Volpe, G.; Delibato, E.; Pucci, E.; Notargiacomo, A.; Pea, M.; et al. Treated Gold Screen-Printed Electrode as Disposable Platform for Label-Free Immunosensing of Salmonella Typhimurium. Electrocatalysis 2019, 10 (4), 288-294. https://doi.org/10.1007/s12678-018-0491-1.
(35) Cardoso, A. R.; Marques, A. C.; Santos, L.; Carvalho, A. F.; Costa, F. M.; Martins, R.; Sales, M. G. F.; Fortunato, E. Molecularly-Imprinted Chloramphenicol Sensor with Laser-Induced Graphene Electrodes. Biosens. Bioelectron, 2019, 124-125 (October 2018), 167-175. https://doi.org/10.1016/j.bios.2018.1.0.015. [incorporated by reference herein].
(36) Ye, Y.; Yan, W.; Liu, Y.; He, S.; Cao, X.; Xu, X.; Zheng, H.; Gunasekaran, S. Electrochemical Detection of Salmonella Using an InvA Genosensor on Polypyrrole-Reduced Graphene Oxide Modified Glassy Carbon Electrode and AuNPs-Horseradish Peroxidase-Streptavidin as Nanotag. Anal. Chim. Acta 2019, 1074, 80-88. https://doi.org/10.1016/j.aca.2019.05.012. [incorporated by reference herein].
(37) Muniandy, S.; Teh, S. J.; Appaturi, J. N.; Thong, K. L.; Lai, C. W.; Ibrahim, F.; Leo, B. F. A Reduced Graphene Oxide-Titanium Dioxide Nanocomposite Based Electrochemical Aptasensor for Rapid and Sensitive Detection of Salmonella enterica. Bioelectrochemistry 2019, 127, 136-144. https://doi.org/10.1016/j.bioelechem.2019.02.005.
(38) Lee, J. A.; Hwang, S.; Kwak, J.; Park, S. II; Lee, S. S.; Lee, K. C. An Electrochemical Impedance Biosensor with Aptamer-Modified Pyrolyzed Carbon Electrode for Label-Free Protein Detection. Sensors Actuators, B Chem. 2008, 129 (1), 372-379. https://doi.org/10.1016/j.snb.2007.08.034.
(39) Ping, J.; Zhou, Y.; Wu, Y.; Papper, V.; Boujday, S.; Marks, R. S.; Steele, T. W. J. Recent Advances in Aptasensors Based on Graphene and Graphene-like Nanomaterials. Biosens. Bioeleciron. 2015, 64, 373-385. https://doi.org/10.1016/j.bios.2014.08.090.
(40) Novoselov, K. S.; Geim, A. K.; Morozov, S. V.; Jiang, D.; Zhang, Y.; Dubonos, S. V.; Grigorieva, I. V.; Firsov, A. A. Electric Field Effect in Atomically Thin Carbon Films. Science (80-.). 2004, 306 (5696), 666-669. https://doi.org/10.1126/science.1102896.
(41) Balandin, A. A. Thermal Properties of Graphene and Nanostructured Carbon Materials. Nat. Mater. 2011, 10 (8), 569-581. https://doi.org/10.1038/nmat3064.
(42) Craciun, M. F.; Russo, S.; Yamamoto, M.; Tanicha, S. Tuneable Electronic Properties in Graphene. Nano Today 2011, 6 (1), 42-60. https://doi.org/10.1016/j.nantod.2010.12.001.
(43) Kurra, N.; Jiang, Q.; Nayak, P.; Alshareef, H. N. Laser-Derived Graphene: A Three-Dimensional Printed Graphene Electrode and Its Emerging Applications. Nano Today 2019, 24, 81-102. https://doi.org/10.1.016/j.nantod.2018.12.003.
(44) Lee, J.-H.; Park, S.; Choi, J.-W. Electrical Property of Graphene and Its Application to Electrochemical Biosensing. Nanomaterials 2019, 9 (2), 297. https://doi.org/10.3390/nano9020297.
(45) Abramova, V.; Slesarev, A. S.; Tour, J. M. Meniscus-Mask Lithography for Narrow Graphene Nanoribbons. ACS Nano 2013, 7 (8), 6894-6898. https://doi.org/10.1021/nn403057t.
(46) Abbas, A. N.; Liu, G.; Liu, B.; Zhang, L.; Liu, H.; Ohlberg, D.; Wu, W.; Zhou, C. Patterning, Characterization, and Chemical Sensing Applications of Graphene Nanoribbon Arrays down to 5 nm Using Helium Ion Beam Lithography. ACS Nano 2014, 8 (2), 1538-1546. https://doi.org/101021/nn405759v.
(47) Wei, D.; Liu, Y.; Wang, Y.; Zhang, H.; Huang, L.; Yu, G. Synthesis of N-Doped Graphene by Chemical Vapor Deposition and Its Electrical Properties. Nano Lett. 2009, 9 (5), 1752-1758. https://doi.org/10.1021/n1803279t.
(48) El-Kady, M. F.; Strong, V.; Dubin, S.; Kaner, R. B. Laser Scribing of High-Performance and Flexible Graphene-Based Electrochemical Capacitors. Science (80-). 2012, 335 (March), 1326-1330. https://doi.org/10.1126/science.1216744.
(49) Lin, J.; Peng, Z.; Liu, Y.; Ruiz-Zepeda, F.; Ye, R.; Samuel, E. L. G.; Yacaman, M. J.; Yakobson, B. I.; Tour, J. M. Laser-Induced Porous Graphene Films from Commercial Polymers. Nat. Common. 2014, 5, 1-8. https://doi.org/10.1038/ncomms6714. [incorporated by reference herein].
(50) Ye, J.; Tan, H.; Wu, S.; Ni, K.; Pan, F.; Liu, J.; Tao, Z.; Qu, Y.; Ji, H.; Simon, P.; et al. Direct Laser Writing of Graphene Made from Chemical Vapor Deposition for Flexible, Integratable Micro-Supercapacitors with Ultrahigh Power Output. Adv. Mater. 2018, 30 (27), 1-8. https://doi.org/10.1.002/adma.201801.384.
(51) Khan, N. I.; Maddaus, A. G.; Song, E. A Low-Cost Inkjet-Printed Aptamer-Based Electrochemical Biosensor for the Selective Detection of Lysozyme. Biosensors 2018, 8 (1). https://doi.org/10.3390/bios8010007.
(52) Torrisi, F.; Hasan, T.; Wu, W.; Sun, Z.; Lombardo, A.; Kulmala, T. S.; Hsieh, G. W.; Jung, S.; Bonaccorso, F.; Paul, P. J.; et al. Inkjet-Printed Graphene Electronics. ACS Nano 2012, 6 (4), 2992-3006. https://doi.org/10.1021/nn2044609.
(53) Suprem, R. Das; Nian, Q.; Cargill, A. A.; Hondred, J. A.; Ding, S.; Saei M.; Cheng, G. J.; Claussen, J. C. 3D Nanostructured Inkjet Printed Graphene: Via UV-Pulsed Laser Irradiation Enables Paper-Based Electronics and Electrochemical Devices. Nanoscaie 2016, 8 (35), 15870-15879. https://doi.org/10.1039/c6nr04310k. [incorporated by reference herein].
(54) Parate K.; Rangnekar, S. V.; Jing, D.; Mendivelso-Perez, D. L.; Ding, S.; Secor, E. B.; Smith, E. A.; Hostetter, J. M.; Hersam, M. C.; Claussen, J. C. Aerosol-Jet-Printed Graphene Immunosensor for Label-Free Cytokine Monitoring in Serum. ACS Appl. Mater. Interfaces 2020, 12 (7), 8592-8603. https://doi.org/10.1021/acsami.9b22183. [incorporated by reference herein].
(55) Secor, E. B.; Prabhumirashi, P. L.; Puntambekar, K.; Geier, M. L.; Elersarn, M. C. Inkjet Printing of High Conductivity, Flexible Graphene Patterns. J. Phys. Chem. Lett. 2013, 4 (8), 1347-1351. https://doi.org/10.1021/jz400644c. [incorporated by reference herein].
(56) Secor, E. B.; Gao, T. Z.; Dos Santos, M. H.; Wallace, S. G.; Putz, K. W.; Hersam, M. C. Combustion-Assisted Photonic Annealing of Printable Graphene Inks via Exothermic Binders. ACS Appl. Mater. Interfaces 2017, 9 (35), 29418-29423. https://doi.org/10.1021/acsami.7b07189. [incorporated by reference herein].
(57) Ye, R.; James, D. K.; Tour, J. M. Laser-Induced Graphene. Ace. Chem. Res. 2018, 51 (7), 1609-1620. https://doi.org/10.1021/acs.accounts.8b00084. [incorporated by reference herein].
(58) Chen, Y.; Long, J.; Zhou, S.; Shi, D.; Huang, Y.; Chen, X.; Gao, J.; Zhao, N.; Wong, C. UV Laser-induced Polyimide to Graphene Conversion: Modeling, Fabrication, and Application. Small Methods 2019. https://doi.org/10.1002/smtd.201900208.
(59) Vanegas, D. C.; Patifio, L.; Mendez, C.; de Oliveira, D. A.; Torres, A. M.; Gomes, C. L.; McLamore, E. S. Laser Scribed Graphene Biosensor for Detection of Biogenic Amines in Food Samples Using Locally Sourced Materials. Biosensors 2018, 8 (2), 42. https://doi.org/10.3390/bios8020042. [incorporated by reference herein].
(60) Garland, N. T.; McLamore, E. S.; Cavallaro, N. D.; Mendivelso-Perez, D.; Smith, E. A.; Jing, D.; Claussen, J. C. Flexible Laser-Induced Graphene for Nitrogen Sensing in Soil. ACS Appl. Mater. Interfaces 2018, 10 (45), 39124-39133. https://doi.org/10.1021/acsami.8b10991. [incorporated by reference herein].
(61) Rahimi, R.; Ochoa, M.; Yu, W.; Ziaie, B. Highly Stretchable and Sensitive Unidirectional Strain Sensor via Laser Carbonization. ACS Appl. Mater. Interfaces 2015, 7 (8); 4463-4470. https://doi.org/10.1021/am509087u.
(62) Singh, S. P.; Li, Y.; Be'Er, A.; Oren, Y.; Tour, J. M.; Arnusch, C. J. Laser-Induced Graphene Layers and Electrodes Prevents Microbial Fouling and Exerts Antimicrobial Action. ACS Appl. Mater. Interfaces 2017, 9 (21), 18238-18247. https://doi.org/10.1021/acsami.7b04863.
(63) Li, L.; Zhang, J.; Peng, Z.; Li, Y.; Gao, C.; Ji, Y.; Ye, R.; Kim, N. D.; Zhong, Q.; Yang, Y.; et al. High-Performance Pseudocapacitive Microsupercapacitors from Laser-Induced Graphene. Adv. Mater. 2016, 28 (5), 838-845. https://doi.org/10.1002/adma.201503333.
(64) Zhang, C.; Xie, Y.; Deng, H.; Tumlin, T.; Zhang, C.; Su, J. W.; Yu, P.; Lin, J. Monolithic and Flexible ZnS/SnO 2 Ultraviolet Photodetectors with Lateral Graphene Electrodes. Small 2017, 13 (18), 1-7. https://doi.org/10.1.002/sm11.201604197.
(65) Tao, L. Q.; Tian, H.; Liu, Y.; Ju, Z. Y.; Pang, Y.; Chen, Y. Q.; Wang, D. Y.; Tian, X. G.; Yan, J. C.; Deng, N. Q.; et al. An Intelligent Artificial Throat with Sound-Sensing Ability Based on Laser induced Graphene. Nat. Commun. 2017, 8, 1-8. https://doi.org/10.1038/ncomms14579.
(66) Nayak, P.; Kurra, N.; Xia, C.; Aishareef, H. N. Highly Efficient Laser Scribed Graphene Electrodes for On-Chip Electrochemical Sensing Applications. Adv. Electron. Mater. 2016, 2 (10), 1-11. https://doi.org/10.1002/aelm.201600185.
(67) Silva, N. F. D.; Magalhães, J. M. C. S.; Barroso, M. F.; Oliva-Teles, T.; Freire, C.; Delenie-Matos, C. In Situ Formation of Gold Nanoparticles in Polymer Inclusion Membrane: Application as Platform in a Label-Free Potentiometric Immunosensor for Salmonella typhimurium Detection. Talanta 2019, 194 (October 2018), 134-142. https://doi.org/10.1016/j.talanta.2018.10.024.
(68) Tehrani, F.; Bavarian, B. Facile and Scalable Disposable Sensor Based on Laser Engraved Graphene for Electrochemical Detection of Glucose. Sci. Rep. 2016, 6, No. 27975. https://doi.org/10.1038/srep27975.
(69) Fenzi, C.; Nayak, P.; Hirsch, T.; Wolfbeis, O. S.; Alshareef, H. N.; Baeumner, A. J. Laser-Scribed Graphene Electrodes for Aptamer-Based Biosensing. ACS Sensors 2017, 2 (5), 616-620. https://doi.org/10.1021/acssensors.7b00066.
(70) Nicholson, R. S. Theory and Application of Cyclic Voltammetry for Measurement of Electrode Reaction Kinetics. Anal. Chem. 1965, 37 (2111), 1351-1355. https://doi.org/10.102/ac60230a016.
(71) Taguchi, M.; Schwalb, N.; Rong, Y.; Vanegas, D. C.; Garland, N.; Tan, M.; Yamaguchi, H.; Claussen, J. C.; McLamore, E. S. PULSED: Pulsed Sonoelectrodeposition of Fractal Nanoplatinum for Enhancing Amperometric Biosensor Performance. Analyst 2016, 141 (11), 3367-3378. https://doi.org/10.1039/c6an00069j.
(72) Zhang, W.; Lei, Y.; Jiang, Q.; Ming, F.; Costa, P. M. F. J.; Alshareef, H. N. 3D Laser Scribed. Graphene Derived from Carbon Nanospheres: An Ultrahigh-Power Electrode for Supercapacitors. Small Methods 2019, 3 (5). https://doi.org/10.1002/smtd.201900005.
(73) McKeen, L. Polyimides. In The Effect of Sterilization Methods on Plastics and Elastomers; Elsevier, 2012; pp 187-204.
(74) Pimenta, M. A.; Dresselhaus, G.; Dresselhaus, M. S.; Cancado, L. G.; jorio, A.; Saito, R. Studying Disorder in Graphite-Based Systems by Raman Spectroscopy. Phys. Chem. Chem. Phys. 2007, 9 (11), 1276-1291. https://doi.org/10.1039/b613962k.
(75) Wang, Q. H.; Jin, Z.; Kim, K. K.; Hamer, A. J.; Paulus, Ci L. C.; Shih, C. J.; Ham, M. H.; Sanchez-Yamagishi, J. D.; Watanabe, K.; Taniguchi, T.; et al. Understanding and Controlling the Substrate Effect on Graphene Electron-Transfer Chemistry via Reactivity Imprint Lithography. Nat. Chem. 2012, 4 (9), 724-732. https://doi.org/10.1038/nchem.1421.
(76) Nguyen, V. T.; Le, H. D.; Nguyen, V. C.; Ngo, T. T. T.; Le, D. Q.; Nguyen, X. N.; Phan, N. M. Synthesis of Multi-Layer Graphene Films on Copper Tape by Atmospheric Pressure Chemical Vapor Deposition Method. Adv. Nat. Sci. Nanosci. Nanotechnol. 2013, 4 (3), 2-7. https://doi.org/10.1088/2043-6262/4/3/035012.
(77) Griffiths, K.; Dale, C.; Hedley, J.; Kowal, M. D.; Kaner, R. B.; Keegan, N. Laser-Scribed. Graphene Presents an Opportunity to Print a New Generation of Disposable Electrochemical Sensors. Nanoscale 2014, 6 (22), 13613-13622. https://doi.org/10.1039/c4nr04221b.
(78) Abdelbasir, S. M.; El-Sheikh, S. M.; Morgan, V. L.; Schmidt, H.; Casso-Hartmann, L. M.; Vanegas, D. C.; Velez-Torres, I.; McLamore, E. S. Graphene-Anchored Cuprous Oxide Nanoparticles from Waste Electric Cables for Electrochemical Sensing. ACS Sustain. Chem. Eng. 2018, 6 (9), 12176-12186. https://doi.org/10.1021/acssuschemeng.8b02510.
(79) Ohk, S. H.; Koo, 0. K.; Sen, T.; Yamamoto, C. M.; Bhunia A. K. Antibody-Aptamer Functionalized Fibre-Optic Biosensor for Specific Detection of Listeria monocytogenes from Food. J. Appl. Microbial. 2010, 109 (3), 808-817. https://doi.org/10.1111/0365-2672.2010.04709.x.
(80) Ding, S.; Mosher, C.; Lee, X. Y.; Das, S. R.; Cargill, A. A.; Tang, X.; Chen, B.; McLamore, E. S.; Gomes, C.; Hostetter, J. M.; et al. Rapid and Label-Free Detection of Interferon Gamma via an Electrochemical Aptasensor Comprising a Ternary Surface Monolayer on a Gold Interdigitated Electrode Array. ACS Sensors 2017, 2 (2), 210-217. https://doi.org/10.1021/acssensors.6b00581. [incorporated by reference herein].
(81) Ruecha, N.; Shin, K.; Chailapakul, O.; Rodthongkum, N. Label-Free Paper-Based Electrochemical Impedance Immunosensor for Human Interferon Gamma Detection. Sensors Actuators, B Chem. 2019, 279, 298-304. https://doi.org/10.1016/j.snb.2018.10.024.
(82) Punbusayakul, N.; Talapatra, S.; Ajayan, P. M.; Surareungchai, W. Label-Free as-Grown Double Wall Carbon Nanotubes Bundles for Salmonella typhimurium Immunoassay. Chem. Cent. J. 2013, 7 (1), 1-8. https://doi.org/10.1186/1752-153X-7-102.
(83) Alexandre, D. L.; Melo, A. M. A.; Furtado, R. F.; Borges, M. F.; Figueiredo, E. A. T.; Biswas, A.; Cheng, H. N; Alves, C. R. A Rapid and Specific Biosensor for Salmonella Typhimurium Detection in Milk. Food Bioprocess Technol. 2018, 11 (4), 748-756. https://doi.org/10.1007/s11947-017-2051-8.
(84) Jia, F.; Duan, N.; Wu, S.; Dai, R.; Wang, Z.; Li, X. Impedimetric Salmonella Aptasensor Using a Glassy Carbon Electrode Modified with an Electrodeposited Composite Consisting of Reduced Graphene Oxide and Carbon Nanotubes. Microchim. Acta 2016, 183 (1), 337-344. https://doi.org/10.1007/s00604-015-1649-7.
(85) Fei, J.; Dou, W.; Zhao, G. Amperometric Immunoassay for the Detection of Salmonella pullorum Using a Screen-Printed Carbon Electrode Modified with Gold Nanoparticle-Coasted Reduced Graphene Oxide and immunomagnetic Beads. Microchim. Acta 2016, 183 (2), 757-764. https://doi.org/10.1007/s00604-015-1721-3.
(86) Thiha, A.; Ibrahim, F.; Muniandy, S.; Dinshaw, I. J.; Teh, S. J.; Thong, K. L.; Leo, B. F.; Madou, M. All-Carbon Suspended Nanowire Sensors as a Rapid Highly-Sensitive Label-Free Chemiresistive Biosensing Platform. Biosens. Bioelectron. 2018, 107 (December 2017), 145-152. https://doi.org/1.0.101.6/j.bios.2018.02.024.
(87) Appaturi, J. N.; Pulingam, T.; Thong, K. L.; Muniandy, S.; Ahmad, N.; Leo, B. F. Rapid and Sensitive Detection of Salmonella with Reduced Graphene Oxide-Carbon Nanotube Based Electrochemical Aptasensor. Anal. Biochem. 2020, 589 (October 2019), 113489. https://doi.org/10.1016/j.ab.2019.113489.
(88) Wang, F.; Wang, K.; Zheng, B.; Dong, X.; Mei, X.; Lv, J.; Duan, W.; Wang, W. Laser-Induced Graphene: Preparation, Functionalization and Applications. Mater. Technol. 2018, 33 (5), 340-356. https://doi.org/10.1080/10667857.2018.1447265.
(89) Farka, Z.; Juřík, T.; Pastucha, M.; Kovář, D.; Lacina, K.; Skladal, P. Rapid Immunosensing of Salmonella Typhimurium Using Electrochemical Impedance Spectroscopy: The Effect of Sample Treatment. Electroanalysis 2016, 28 (8), 1803-1809. https://doi.org/10.1002/elan.201600093.
(90) Nguyen, P. D.; Tran, T. B.; Nguyen, D. T. X.; Min, J. Magnetic Silica Nanotube-Assisted Impedimetric immunosensor for the Separation and Label-Free Detection of Salmonella typhimurium. Sensors Actuators, B Chem. 2014, 197, 314-320. https://doi.org/10.1016/j.snb.2014.02.089.

TABLE 1
Comparison among different electrochemical biosensors for Salmonella spp. detection.

| | | | | Working | | | | |
|---|---|---|---|---|---|---|---|---|
| | | Detection | LOD | range | Analysis | | | |
| Transducer | Material | Technique | (CFU mL⁻¹) | (CFU mL⁻¹) | time | | Sample | Ref. |
| GCE | rGO | DPV | 10¹ | 10¹-10⁸ | 1 | h | Chicken in | ³⁷ |
| | | | | | | | BPW | |
| ISE | AuNPs | P | 6 | 10¹-10⁶ | 1 | h | PBS, Apple | ⁶⁷ |
| | | | | | | | juice | |
| SPE | rGO | EIS | 10¹ | — | — | | Water and | ²⁰ |
| | | | | | | | Juice | |
| | | | | | | | samples | |
| SP-IDME | Gold | EIS | 10³ | 10³-10⁶ | <2 | h | Chicken | ²³ |
| | | | | | | | rinse water | |
| SPE | Gold | EIS | — | 10³-10⁷ | 90 | min | Redox | ³⁴ |
| | | | | | | | solution | |
| SPE | Gold | EIS | 10³ | 10³-10⁸ | 20 | min | PBS, Milk | ⁸⁹ |
| SPE | Gold | CA | 21 | 10¹-10⁷ | — | | Chicken in | ³¹ |
| | | | | | | | liquid | |
| | | | | | | | samples | |
| DWE | CNTs | CA | 9 | 10²-10⁷ | 6 | h | PBS | ⁸² |
| SPE | AuNPs/rGO | DPV | 89 | 10²-10⁶ | — | | Chicken | ⁸⁵ |
| | | | | | | | liver | |
| GCE | rGO/MWCNTs | EIS | 25 | 75-10⁵ | 1 | h | Chicken | ⁸⁴ |
| IME | Gold/MSNTs | I | 500 | 10³-10⁷ | 30 | min | PBS | ⁹⁰ |
| SPE | Gold | CA | 10 | 10¹-10⁵ | 125 | min | Milk | ⁸³ |
| | | | | | | | Beef in | |
| CE | CNWs | i-V | 10 | 10¹-10³ | 5 | min | BPW | ⁸⁶ |
| | | | | | | | Chicken in | |
| GCE | rGO-CNTs | DPV | 10 | 10¹-10⁸ | 10 | min | BPW | ⁸⁷ |
| LIG | Multilayers | EIS | 10 | 10¹-10³ | 22 | min | BPW | This |
| | Graphene | | | | | | | work |
| LIG | Multilayers | EIS | 13 | 10¹-10⁵ | 22 | min | Chicken | This |
| | Graphene | | | | | | Broth | work |

Glassy Carbon Electrodes (GCE), Carbon Electrode (CE), Ion Selective Electrodes (ISE), Screen-Printed Electrodes (SPE), Screen-Printed Interdigitated Microelectrode (SP-IDME), Double-Walled Electrode (DWE), Interdigitated Microelectrode (IME), Reduced Graphene Oxide (rGO), Gold Nanoparticles (AuNPs), Carbon Nanotubes (CNTs), Multi-Walled Carbon Nanotubes (MWCNTs), Magnetic Silica Nanotubes (MSNTs), Carbon Nanowires (CNWs) Buffered Peptone Water (BPW), Phosphate Buffered Saline (PBS), Differential Pulse Voltammetry (DPV), Potentiometry (P), Chronoamperometry (CA), Impedance (I), Current-Voltage (i-V).

It can therefore be seen that the foregoing Specific Embodiment 1 meets or exceeds at least one or more of the objects, features, advantages, and aspects of the invention. This example includes proof-of-concept data regarding this embodiment and its examples.

### 2. Specific Embodiments and Examples 2-4

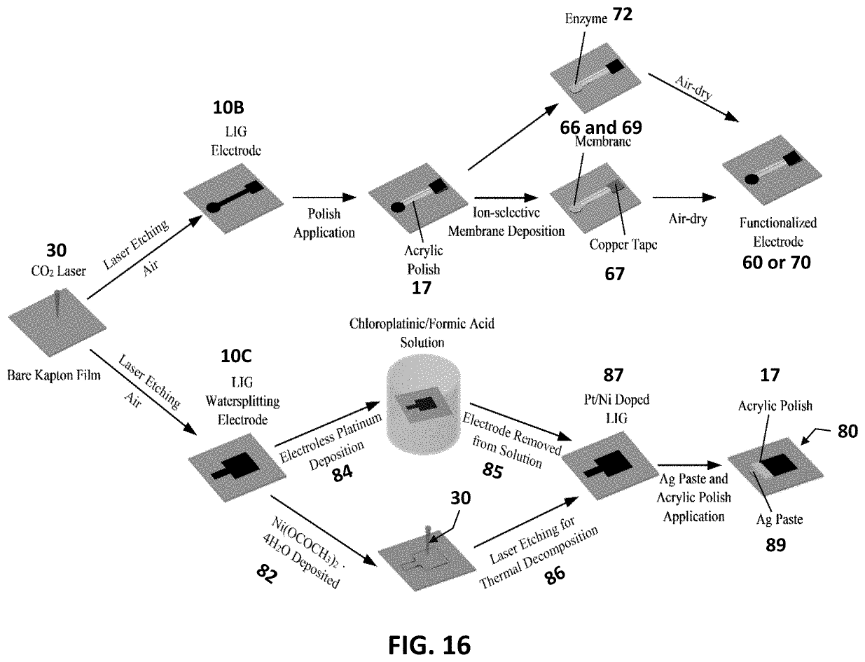

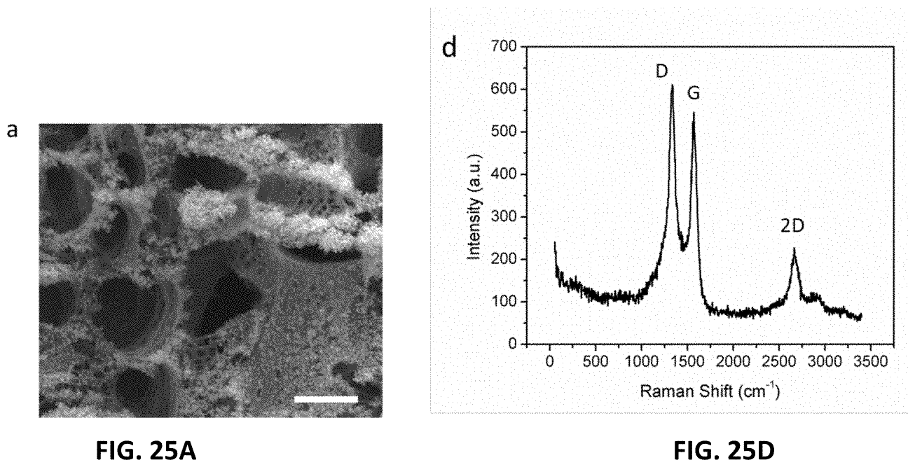

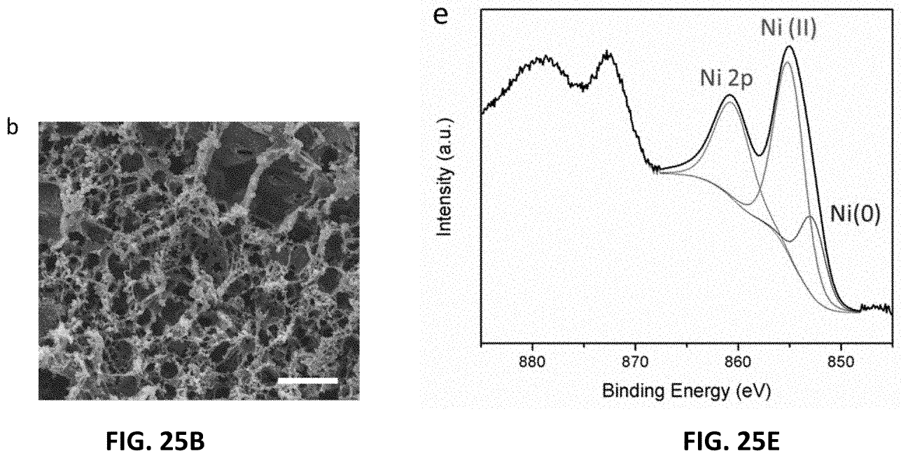

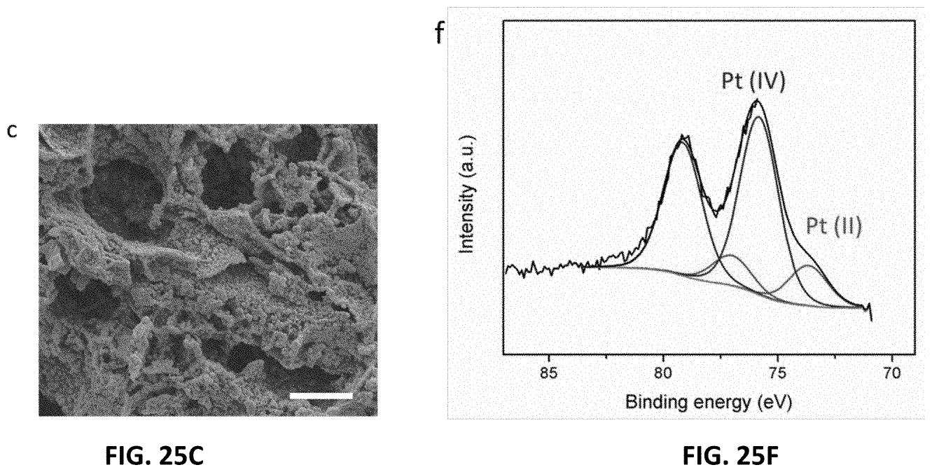

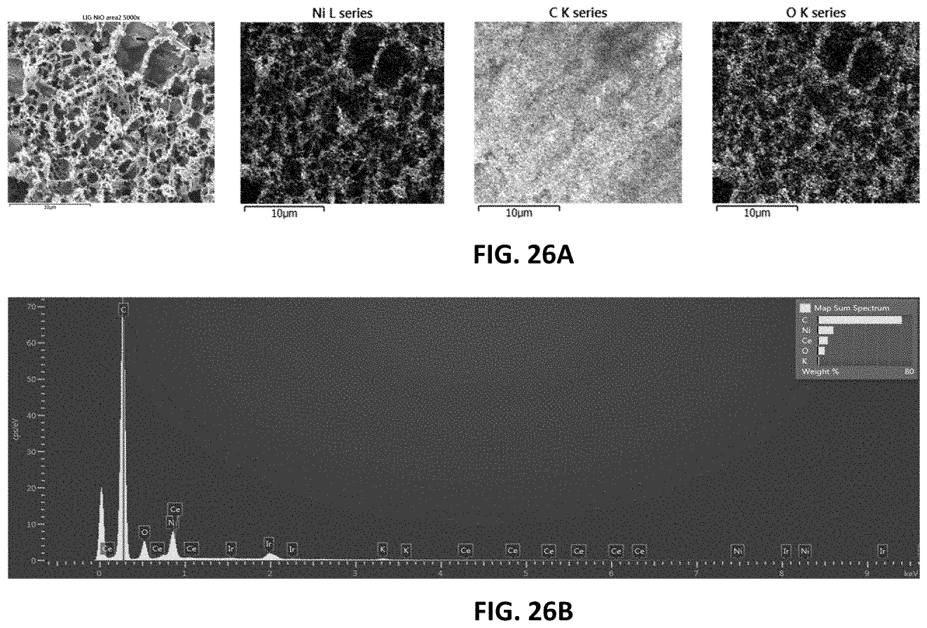

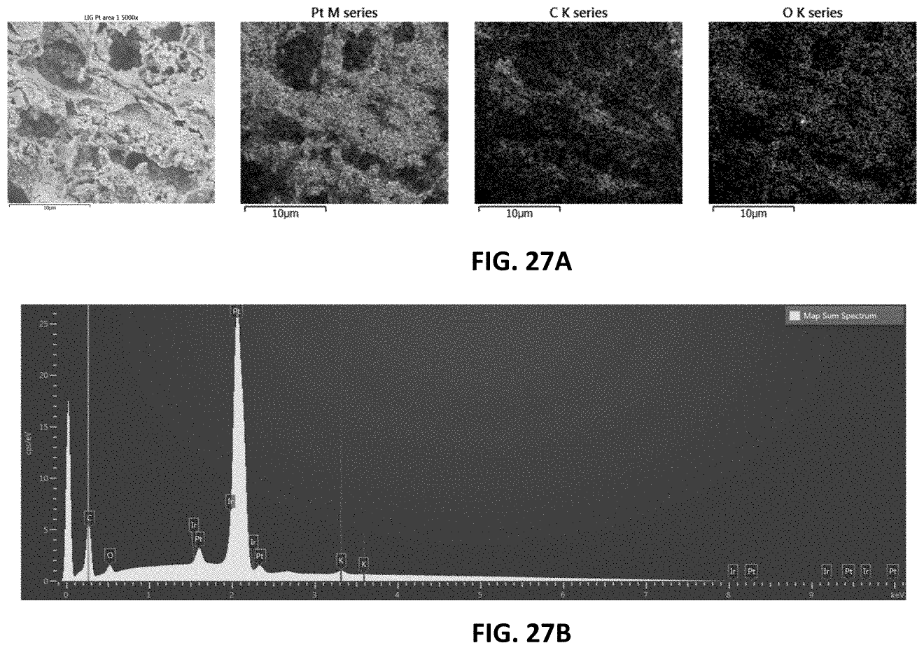

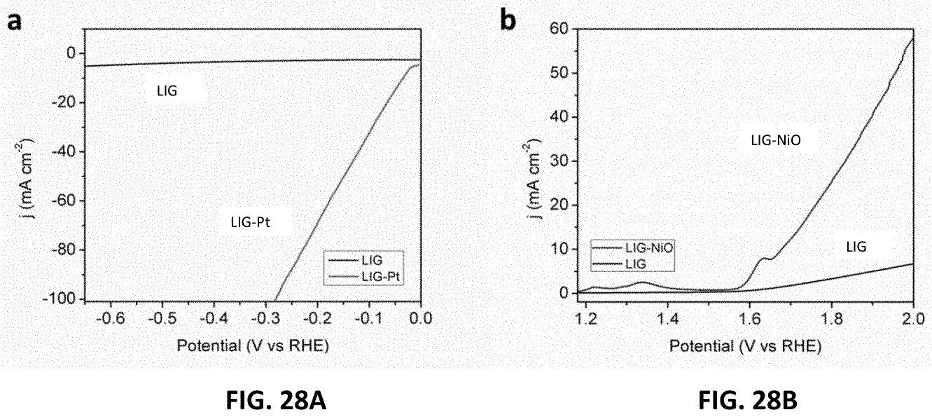

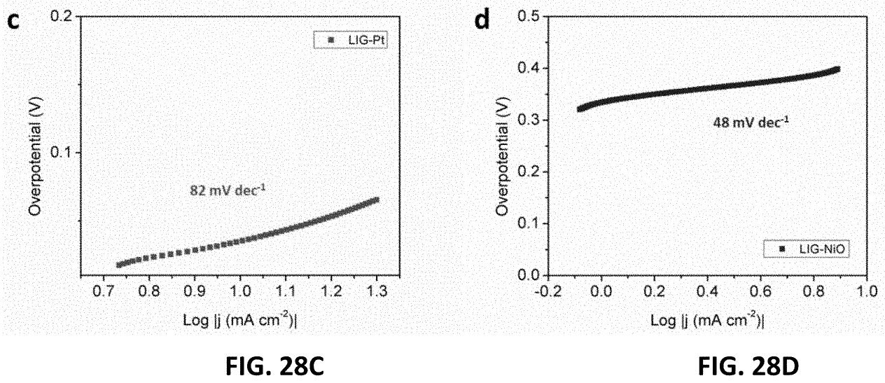

With particular reference to FIGS. 16 to 20A-D and FIGS. 25A-F to 28A-D, additional specific embodiments of apparatus, methods, and systems according to the invention are set forth in more detail. In particular, these examples utilize LIG electrodes like those in Specific Example 1, supra, but configured for different applications, as discussed below.

### Laser-Induced Graphene Electrodes for Electrochemical Ion Sensing, Pesticide Monitoring, and Water Splitting

### Abstract

Laser induced graphene (LIG) has shown to be a scalable manufacturing route to create graphene electrodes that overcomes the expense associated with conventional graphene electrode fabrication. Herein we expand upon Specific Example 1, supra, by functionalizing the LIG with metallic nanoparticles for ion sensing (Specific Example 2), pesticide monitoring (Specific Example 3), and water splitting (Specific Example 4). The LIG electrodes were converted into ion-selective sensors by functionalization with poly(vinyl chloride)-based membranes containing K⁺ and H⁺ ionophores. These ion selective sensors exhibited a rapid response time (10-15 s), near-Nernstian sensitivity (53.0 mV/dec for K⁺ sensor and −56.6 mV/pH for pH sensor), long storage stability for 40 days, and were capable of ion monitoring in artificial urine. The pesticide biosensors were created by functionalizing the LIG electrodes with the enzyme horseradish peroxidase and displayed a high sensitivity to atrazine (28.9 mA/μM) with negligible inference from other commons herbicides (glyphosate, dicamba and 2,4-dichlorophenoxyacetic acid). Finally, the LIG electrodes also exhibited small overpotential for hydrogen evolution reaction and oxygen evolution reaction. The OER tests yielded overpotentials of 448 my and 995 mV for 10 mA/cm² and 100 mA/cm², respectively. The HER tests yielded 35 mV and 281 mV for the corresponding current densities. Such versatile LIG platform paves the way for simple, efficient electrochemical sensing and energy harvesting applications.

### Introduction

Graphene is a one-atom-thick sheet of carbon atoms arranged in a honeycomb-like structure. It is characterized by large surface area, high electrical conductivity, high mechanical stiffness and thermal conductivity¹. Such properties make graphene-based electrodes particularly attractive for various electrochemical applications, including ion sensing, amperometric sensing, and water splitting^{2,3}. Traditional methods for the creation of graphene-based materials generally includes chemical vapor deposition (CVD) which is a laborious process that requires vacuum chambers and high temperatures (500-1000° C.)⁴. Furthermore, after such CVD synthesis, additional complex procedures is often required to transfer the synthesized graphene to insulating dielectric materials that are suitable for electronic devices^{5,6}. To simplify graphene-based circuit fabrication, researchers have developed a variety of printing techniques including inkjet^{7,8}, screen⁹, and dispenser printing¹⁰. Though these techniques generally print exfoliated flakes of graphite (graphene or graphene oxide) that are less conductive then pristine CVD-grown graphene, researchers have developed a variety of post-print annealing techniques (i.e., rapid pulse laser annealing, high-temperature thermal annealing, and photonic annealing) that improves the electrical conductivity to levels near printed metals (<1k Ω)^{7,11-13}. Recently researchers have been able to effectively combine direct write printing and post-print annealing to create highly conductive graphene circuits via a fabrication method coined laser-induced graphene (LIG) or laser scribed graphene (LSG)¹⁴⁻¹⁷. LIG can be produced from carbon-based polymers (i.e., polyimide) by direct write CO₂ laser irradiation which converts the hybridization of carbon within the polymer from sp³ to sp² found in graphene¹⁵. LIG circumvents the need for ink formulation and post-print annealing involved in graphene printing techniques, uses inexpensive raw materials, and can create electrode designs on-the-fly as circuits can be rapidly created with CAD software and uploaded into the CO₂ laser.

Researchers have been able to functionalize the surface of LIG with nanoparticles and/or biorecognition agents to create a wide variety of electrodes which include those capable of splitting water and water oxidation^{18,19}, sensing fertilizer and hydrations ions in soil and sweat respectively^{20,21}, monitoring of biogenic amines and Salmonella bacteria in food samples^{16,22}, impedance-based cell monitoring²³, aptamer-based biosensing in serum¹⁷, and biomarkers in sweat²⁴. In this section, we will expand upon these reports by demonstrating a simpler approach to creating LIG electrodes for water splitting, expanding the research of LIG-based ion sensing to include pH monitoring, and introducing the first example of a pesticide monitoring LIG-based electrode through functionalization with an enzyme.

Potentiometric ISEs have been used for the selective and sensitive (nanomolar detection limits) detection of ions in a wide variety of applications including detecting perchlorate and iodide in water^{25,26}, ammonium and potassium in urine²⁷, and lithium in whole blood^{28,29}. Conventional liquid-junction ISEs contain an aqueous internal reference electrolyte between the working electrode and ion selective membrane that requires maintenance and creates long-term stability due to leeching. However, solid-contact or solid-state ISEs do not contain such a liquid reference electrolyte which can improve their stability and decrease their maintenance³⁰. In the present work, we develop solid-state ISEs for the detection of K⁺ and pH (pH ISE is in fact a sensor for H⁺). These targets were selected since their detection plays an important role in environment sensing³¹, human health monitoring²⁷, and medical diagnostics (e.g. for use on catheter tips or in implantable devices)³². The created K⁺ and pH ISEs have a wide linear range (0.3-150 mM K⁺ and pH 5-8), near-Nernstian sensitivity (53.0 mV/dec for K⁺ ISE and −56.6 mV/pH for pH ISE), and excellent storage stability during at least 40 days.

In addition to ion sensing, we demonstrate how LIG-based electrodes can be used for pesticide monitoring through functionalization with the enzyme horseradish peroxidase (HRP). Horseradish peroxidase (IMP), a helve group enzyme has shown selective measurement of hydrogen peroxide through amperometry^{33,34}. Electrodes functionalized with HRP have been used to monitor a wide variety of analytes including glyphosate, atrazine, and dichlofenthion³⁵⁻³⁷. However, we demonstrate that LIG electrodes functionalized with HRP can selectively monitor the herbicide atrazine with high sensitivity (28.9 mA/μM) and report minimal interference from other herbicides commonly applied in the Corn Belt region of the United States including glyphosate, dicamba, and 2,4-dichlorophenoxyacetic acid.

Finally, we demonstrate how LIG-based electrodes can be used in electrochemical energy conversion. Electrochemical electrolysis has gained recent attraction due to its ability to increase hydrogen evolution reaction (HER) and oxygen evolution reaction (OER) kinetics³⁸⁻⁴⁰. To date, various noble metals and semiconductors have been promising as HER catalysts. Phosphides^{41,42}, sulfides, and manganese has shown good performance in HER. Hydroxides/oxides of nickel⁴³, iron^{43,44}, cobalt⁴⁵ has shown good performance in OER. However, metallic platinum (Pt) remains as one of the most promising used catalysts for electrolysis due to its high catalytic activity in liberating H₂ at high reaction rates and low overpotentials (η). To reduce the cost but maintain the superior catalytic activity, nanoscale amounts of platinum in the form platinum nanoparticles (PtNPs) are often deposited onto the electrodes via drop-cast⁴⁶, thermal decomposition of Pt salt^{18,47}, and atomic layer deposition⁴⁸. However, recently researchers have demonstrated that electroless deposition of PtNPs is a simple one-pot synthesis process offering several advantages compared to commonly used methods to synthesis electrocatalysts (such as solid-phase reaction and hydrothermal, which require delicate control of reaction time and temperature. The developed LIG-Pt and LIG-NiO electrodes display a high overpotential 100 mA/cm² for HER, and OER is as low as 281 and 995 mV with relatively low Tafel slopes of 82 and 48 mV/dec, respectively.

### Materials and Methods

### Fabrication of Laser-Induced Graphene Electrodes

The LIG electrodes were prepared by irradiation of polyimide (0.125 mm thick, DuPont, USA) by the CO₂ laser (75-watt Epilog Fusion M2, USA). The polyimide was fixed on a glass substrate and cleaned with a wipe before lasing. Drawings of the electrodes were prepared in CorelDraw and then submitted to the laser-controlling program. Lasing parameters were the following: 7% speed, 7% power, 50% frequency, raster mode, 600 dpi, and all other parameters were selected as “off”. The laser beam was defocused by the placement of polyimide sheet 2 mm lower than the focus distance. These conditions were experimentally selected to achieve the highest quality of graphene.

### Preparation of Water Splitting Electrodes

Preparation of LIG-NiO electrodes. Once LIG electrodes were lasered, 15 μL of 1M nickel (II) acetate tetrahydrate was deposited onto each electrode. The solution was allowed to dry for 10 minutes. The CO₂ laser, running the same etching pattern, lasered the LIG a second cycle to induce the thermal decomposition of the nickel salt into nickel (II) oxide and other gases, such as oxygenates, hydrogen with hydrocarbons, and carbon oxides. The silver paste was applied to the stem of the LIG electrode. Acrylic polish was applied to the electrode to ensure a constant, working surface area of 25 mm² square throughout experiments.

Preparation of LIG-Pt electrodes. The deposition of Pt on LIG is followed by the recipe reported previously⁴⁹⁻⁵². A chloroplatinic/formic acid solution with a 100:5 ratio, respectively, was used to electrolessly deposit Pt onto bare LIG electrodes. Bare electrodes were placed into a glass vial containing 10 mL, of the acid solution. The pH of the solution was changed from 1.68 to 1.75 by adding Ammonium Hydroxide in order to increase the rate of Pt deposition. The electrodes sat in the solution for 24 hours under ambient conditions. In a similar manner to the LIG-NiO electrode, silver paste and acrylic polish were applied.

### Preparation of Ion-Selective Electrodes

Mixture for the preparation of K⁺ ISE contained valinomycin (potassium-selective ionophore, 2.5 mg), dioctyl sebacate (plasticizer, 165 mg), poly(vinyl chloride) high molecular weight (the main solid component of the membrane, 82.5 mg), and tetrahydrofuran (solvent, 1667 mg)⁵³. Mixture for the preparation of pH ISE contained hydrogen ionophore I (5 mg), potassium tetrakis(4-chlorophenyl)borate (ionic additive for better ion exchange, 2.5 mg), dioctyl sebacate (327.5 mg), poly(vinyl chloride) high molecular weight (165 mg), and tetrahydrofuran (3163 mg)⁵⁴. After preparation, the mixtures were vortexed for 3 min, left in the fridge (+4° C.) overnight for a complete dissolution of the components and vortexed again for 3 min before usage. The mixtures could be used for several months if stored in the fridge. However, to achieve the best sensor performance it is better to prepare new mixtures every 2-3 weeks. K⁺ and pH ISEs were prepared by drop-casting of 8 μL of the corresponding ionophore mixture onto the terminal circular part of the electrode. About 1 mm of the surrounding area (polyimide) was also covered with the mixture to prevent direct contact between the tested solution and the graphene. For the same reason, the central part of the electrode was covered in advance with acrylic polish. The prepared ISEs were stored dry at room temperature.

### Preparation of Horseradish Peroxidase—LIG Electrodes

Preparation of Horseradish Peroxidase—LIG electrodes. Once LIG electrodes were lasered, acrylic polish was applied to the stem of the electrode to act as a passivation layer. Silver paste was applied to the contact of the electrode. A solution of 3% Horseradish Peroxidase (HRP) Type VI (Sigma Aldrich) and 2% Glutaraldehyde (weight) was prepared fresh in a 1:1 volumetric ratio. After mixing this solution, 1 μL of the mixture was drop-coated onto the pad of the working electrode. The deposited enzyme mixture on the electrode was allowed to dry for 12 hours at 4° C. before testing.

### Preparation of Artificial Urine

Artificial urine for testing of K+ and pH ISEs was prepared according to⁵⁵, with few modifications. The artificial urine was obtained by dissolving the following components in DI: sodium L-lactate (1.1 mM), sodium citrate dehydrate (2 mM), sodium bicarbonate (25 mM), urea (170 mM), uric acid (0.4 mM), calcium chloride (2.5 mM), sodium chloride (90 mM), magnesium sulphate (2 mM), sodium sulphate (10 mM), sodium dihydrogen phosphate (7 mM), disodium hydrogen phosphate (7 mM). The solution was filtered through a 45 μM syringe filter to remove larger undissolved components. Thus, all ions found endogenously in urine were present in the artificial urine except potassium and ammonium. The pH of the as-prepared artificial urine was 8.5 and it was decreased to lower values (pH 5, 6, 6.5, 7, 7.5, 8) using hydrochloric acid (HCl).

### Electrochemical Characterization

Water splitting electrodes. A three-electrode setup was used on a CHI 6273E electrochemical analyzer (potentiostat), with a Pt wire as the counter electrode, Ag/AgCl as the reference electrode, and LIG as the working electrode. The tests were performed in 1M KOH. Linear Sweep Voltammetry (LSV) was performed for both the LIG-NiO and LIG-Pt electrodes. The LIG-Ni electrode was tested for the Oxygen Evolution Reaction (OER) from 0 to 1.7 V (vs Ag/AgCl) at a scan rate of 5 mV/s. The LIG-Pt electrode was tested for the Hydrogen Evolution Reaction (HER) from 0 to −1.7V (vs Ag/AgCl) at a scan rate of 5 mV/s.

Ion-selective electrodes. The developed LIG ISEs were electrochemically characterized with a CHI 6273E electrochemical analyzer (potentiostat). The potentiostat operated in “open circuit potentiometry” mode, with 0.5 s interval between separate readings. An ISE was connected to the working connector, and a reference electrode (CHI111 Ag/AgCl liquid-junction reference electrode filled with 1 M KCl) was connected to combined reference and counter connectors. The ISE and the reference electrode were placed in a 10 mL glass beaker filled with 4 mL of DL with constant slow stirring (˜100 RPM) by a magnetic stirrer. For the sensor calibration, aliquots of concentrated KCl solution (1, 10, 100, and 2000 mM in DI) were added to the working cell. Calibration of pH ISEs was carried out in standard pH solutions with pH 4, 7, and 10 (Fisher Scientific) and in 1 mM phosphate buffer with different pH levels adjusted with HCl and NaOH.

Horseradish Peroxidase—LIG electrodes. A three-electrode setup was used on a CHI 6273E electrochemical analyzer with a platinum wire counter electrode, Ag/AgCl reference electrode, and functionalized LIG working electrode. Amperometric i-t tests were performed at −0.1 V. The test cell contained phosphate buffer saline pH 7.4. A calibration experiment for atrazine was conducted by starting the i-t test. Upon reaching steady state, 0.4 mM hydrogen peroxide was added to the test cell. As the oxidation-reduction reaction occurred between HRP and H₂O₂, a signal was detected. Once this signal reached steady state, atrazine was added in 10 μM increments, allowing a steady state signal to be reached before the next addition. The test cell contained PBS (pH 7.4). The same three-electrode setup was used. An i-t test was performed for a single sensor by testing the response to each pesticide at 10 μM additions. The test setup was rinsed 2 times with PBS (pH 7.4) at the end of every test. The same sensor and procedure were used to test for the detection of dicamba, 2,4-dichlorophenoxyacetic acid, and glyphosate.

Microscopy Characterization Microscopic images are taken from a FEI Quanta 250 FEG Scanning Electron Microscope (SEM) at an operating voltage of 10 kV with EDS. An Oxfords Instruments Aztec X-Max 80 detector system connected to FEI Quanta 250 was used to collect the EDX photons for elemental detection. Raman spectrum was obtained by using an XploRa Plus confocal Raman upright microscope equipped with a 532 nm excitation source and a Synapse Electron Multiplying Charge Coupled Device camera (Horiba Scientific/NJ, France). The intensity (height) was determined for the D, G, and 2D bands by fitting the data to a Lorentzian model. XPS measurements were performed using a PHI ESCA 5500 instrument. The sample was irradiated with 200 W unmonochromated Al Kα X-rays. CasaXPS was used to process raw data files.

### Results and Discussion

### Characterization of the LIG Electrodes

The LIG electrodes 10B and 10C used for these tests were produced by etching 0.005″ polyimide (PI) with a CO₂ laser 30 as shown in FIG. 16A. After lasing, a dielectric coating 17 was applied on the sample with only the disk-shape working area (dia. 5 mm) exposed. The ISE 60 were prepared by drop-coating ion selective membranes 66 and 69 selective to K⁺ and pH on the working area followed by conditioning overnight to stabilize the sensor and improve detection limit²⁸. The HRP sensors 70 were prepared by drop-coating enzyme mixture 72 on working electrode followed by drying in air for 12 hours. The fabrication of water splitting electrode 80 includes thermal decomposition of nickel (II) acetate tetrahydrate 82 and an electroless deposition method for the platinum (Pt) 84, 85. Refer to the experimental section for a summary of the fabrication process.

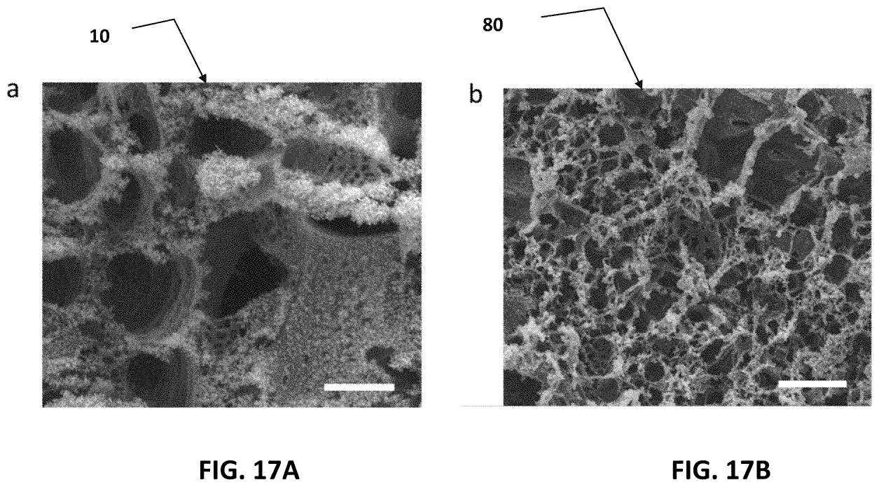

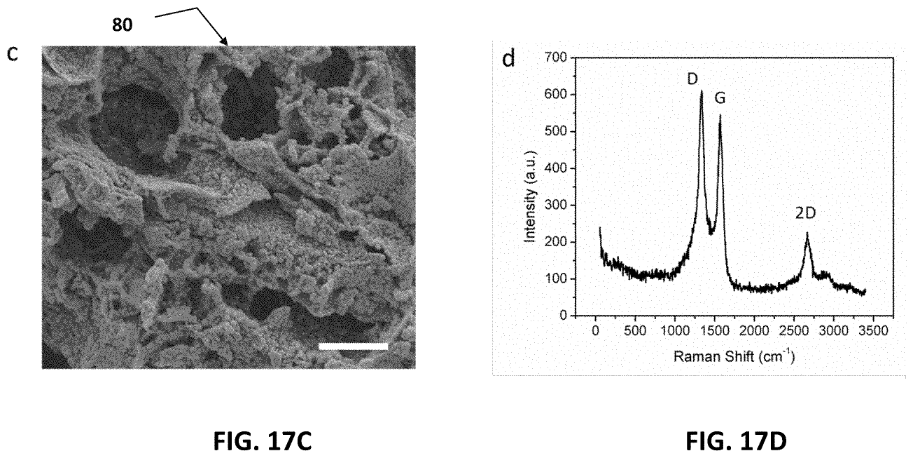

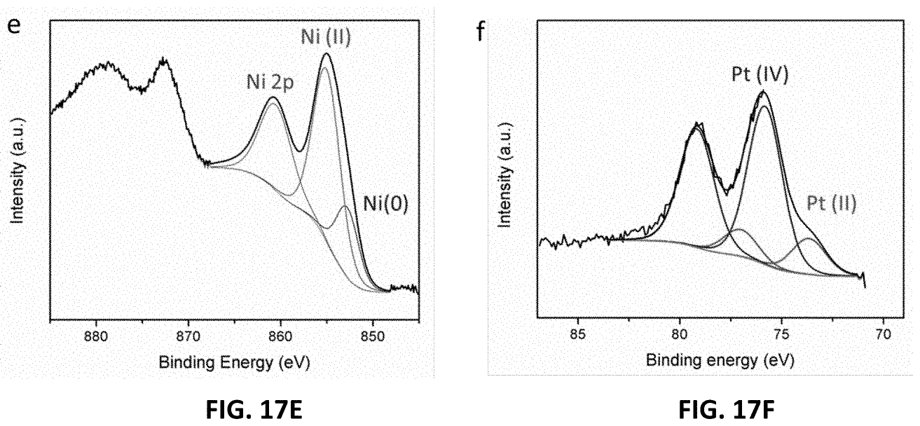

Scanning electron microscopy (SEM) images with corresponding EDS were acquired to investigate the morphology of the LIG electrodes. The pristine LIG 10 (FIG. 17A) show porous structure with pores ranging from 200 nm to 10 μm. FIG. 17B shows the structure of the nickel (II) deposition on the graphene. Similarly, FIG. 17C shows the structure of the platinum (II) deposition. Thermal decomposition of nickel acetate tetrahydrate into nickel oxide (NiO) requires a temperature of at least 380° C. The LIG-NiO also shown an increase of porosity on FIG. 17B, which could be attributed to the thermal reduction of graphene during the deposition of NiO. This was not observed on the Pt-LIG sample as shown in FIG. 17C. The corresponding EDS is also performed to confirm the element presented in the NiO-LIG sample as shown in FIG. 25A, the main elements presented are carbon, oxygen and nickel. The carbon can be found across the sample confirmed the successfully convert of PI into LIG by laser inducing. The nickel and oxygen can be found in the same area which confirm the presence of NiO on the LIG surface.

Pt was electrolessly deposited onto the LIG by immersing the sample into chloroplatinic acid solution following the protocol that was previous developed in our group⁴⁹⁻⁵¹. The method can deposit platinum onto various surface such as carbon based (carbon nanotube, carbonized wood, graphene) and others like cellulose and Sift wools with surface functionalization⁵⁰⁻⁵². After about 20 hrs, a layer of Pt is deposited onto the surface of LIG as shown in FIG. 17C. The EDS is also conduct on Pt-LIG sample to confirm the presence of Pt deposition as shown in FIGS. 25A-F. The Raman characterization was performed to confirm the presence of graphene in LIG (FIG. 17D). Three prominent peaks are observed on the LIG, the D peak at ˜1,350 cm⁻¹ induced by defects or bent sp²-carbon bonds, the first-order allowed G peak at ˜1,580 cm⁻¹ and the 2D peak at ˜2,700 cm⁻¹ originating from second order zone-boundary phonons⁵⁶. The intensity (height) was determined for the D, G, and 2D bands by fitting the data to a Lorentzian model. The presence of this 2D band in the Raman spectra suggests graphitization of the polyimide substrate and formation of graphene layers due to the high local heating by CO₂ Laser. It shows the I_{D}/I_{G} ratio of 1.2±0.2 and I_{2D}/I_{G} ratio of 0.28±0.04. XPS was also performed on water splitting catalyst to confirm the elemental composition and chemical valance states. For the LIG-NiO, a mixture of metallic Ni and Ni²⁺ is presented with the main peaks at ˜852.8 eV, ˜855.03 eV and ˜860.53 eV. The metallic Ni is about 21% and the Ni²⁺ is about 79% (FIG. 17F). The presence of metallic Ni could be attributed to the reduction of Ni²⁺ at elevated temperasture¹⁹. For the LIG-Pt, a mixture of Pt in the +2 and +4 states is presented with the main peak at ˜73.65 eV; ˜77 eV for Pt(II) and ˜75.82 eV; ˜79.17 eV for Pt(IV) (FIG. 17E). The +2 state has about 18% and the +4 state 82%. They could be PtO and PtO₂.

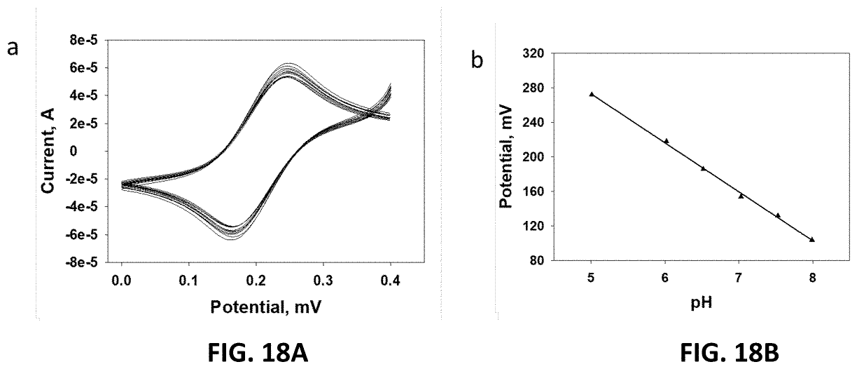

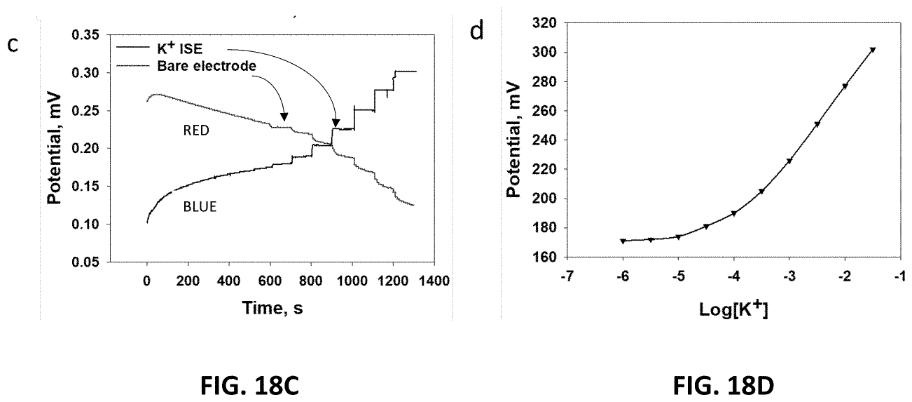

Electrochemical performance of LIG were characterized by cyclic voltammetry in 5 mM ferricyanide-ferrocyanide mixture. Eleven electrodes from the same batch were tested (FIG. 18A), and showed similar shape of voltammograms and peak currents with little batch-to-batch variation. Peak-to-peak separation was about 90 mV, which is similar or better than achieved with other carbon nanomaterials and indicates excellent electrochemical performance of the electrode. For example, the following peak-to-peak separations were reported for electrodes based on various carbon nanomaterials: 68 mV (pyrolytic graphite)⁵⁷, 81 mV (LIG)⁵⁸, 90 mV (single-walled carbon nanotubes modified with Pt nanospheres)⁵⁹, 92 mV (single-walled carbon nanotubes modified with Pd nanocubes)⁵⁹, 96 mV (single-walled carbon nanotubes)⁶°, 183 mV (pyrolytic graphite)⁵⁷, 230 mV (multi-walled carbon nanotubes)⁶⁰, 630 mV (pyrolytic graphite)⁶¹.

Other advantages of the developed LIG electrodes are fast and easy production process (it takes 12-15 s to lase one electrode) and low cost of materials (cost of polyimide sheet for one electrode is $0.02). For comparison, commercial CVD-grown graphene film (1×1 cm², 4 items) cost $352 (Sigma-Aldrich, ref. #773719). Recently reported electrode based on carbon nanotubes, characterized by the authors as simple and fast in production, requires 5-step preparation process that lasts ˜6-8 h⁶². Graphene modified with MoS₂ nanocrystals was synthesized 45-second microwave annealing of reaction mixture (MoCl₅, graphene oxide, thiourea, and ethanol) using conventional microwave oven⁶³.

### Potassium (K⁺) and pH Ion Selective Sensors

Calibration of K⁺ ISEs was carried out in DI by sequential increase of KCl concentration in the working cell (FIG. 18C, labelled “blue” line). As a control, bare LIG electrode was also calibrated (FIG. 18C, labelled “red” line) As seen, K⁺ ISE responds rapidly after addition of KCl, and response time is 10-15 s. The response is going to the positive direction since K⁺ cations bind with the ionophore and increase potential of the ion-selective membrane. On the contrary, the bare electrode does not show stable response to KCl and demonstrate decrease of potential probably due to higher binding of Cl⁻ anions to the graphene surface.

Typical calibration curve of the K⁺ ISE is shown on FIG. 18C. The linear range of K⁺ detection is from 0.3 mM to at least 150 mM, and the average sensitivity is 53.0 mV/dec. Limit of K⁺ detection is 0.1 mM. We did not check K⁺ concentrations above 150 mM since they usually cannot be found in real samples. The presented characteristics allow the detection of any K⁺ concentration found in urine. FIG. 18D is a typical calibration curve for pH ISE.

Storage stability of the sensors was also investigated. For this experiment K⁺ ISEs were calibrated after preparation and after 40 days of dry storage at room temperature (20-22° C.). Sensitivity of the sensors remained the same, which indicates excellent storage stability. The characteristics of the developed K⁺ ISE are similar to other solid-state electrodes found in literature. For example, solid-state K⁺ ISE based on graphene-covered paper electrode had sensitivity 57 mV/dec²¹. Another sensor based on graphene-coated glassy carbon electrode had sensitivity 59.2 mV/dec and was stable during 3 weeks⁶⁴. Sensitivity of K⁺ ISE based on fullerene-modified glassy carbon electrode was 55 mV/dec⁶⁵.

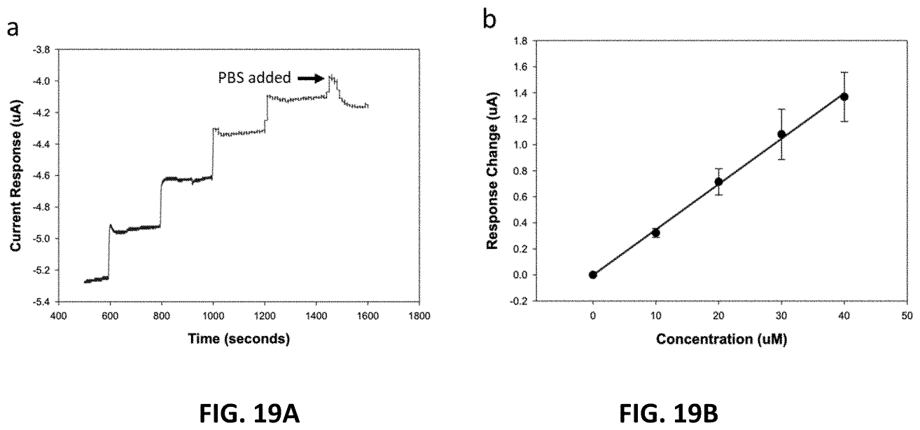

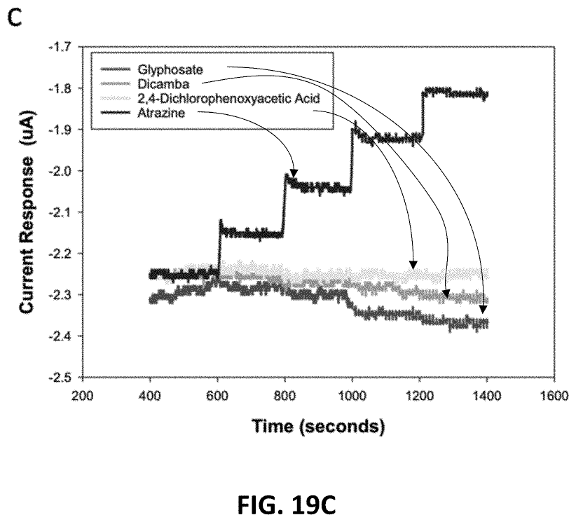

Calibration of pH ISE (see FIGS. 19A-C) was carried out in a wide range buffer (5 mM tris, 5 mM NaH₂PO₄, 5 mM sodium citrate, 5 mM boric acid, pH adjusted with HCl/NaOH) (FIG. 19C). Decrease of pH level leads to the decrease of the ISE potential due to the lower amount of Ft in the ion-selective membrane. The response was linear within pH 5-8 and the calibration curve was described by the equation y=−56,632x+556.15. Thus, the sensitivity of the sensor was −56.6 mV/pH. Storage stability of the sensors was investigated in the same way as for K⁺ ISEs. After 44 days of dry storage sensitivity of the sensors decreased by 1.5 mV/pH. Thus, the pH ISEs were a little less stable than the K⁺ ISEs but still could be efficiently used after storage.

The obtained results appear to approve upon existing solid-state pH sensors. For example, pH ISE based on Pt electrode with PEDOT polymer had initial sensitivity of −46 mV/pH, which decreased by 55% after 28 days of storage⁶⁶. Another pH ISE based on Pt electrode with poly(aniline) had sensitivity of 55-59 mV/pH (depending on ionophore) and remained stable for 1 month⁶⁷. pH ISE based on graphite rod demonstrated sensitivity −55 mV/pH and was stable during 2 month⁶⁸. Graphene-based pH ISE had sensitivity −56 mV/pH, but its storage stability was not tested²¹.

### Measurements of K⁺ Concentration and pH in Artificial Urine

To evaluate performance of the sensors in conditions close to real applications we analyzed artificial urine, prepared as described in Materials and Methods. Five samples of artificial wine with different pH and K⁺ content were used; the pH and K⁺ concentrations were chosen from their typical values in urine (reference range for K⁺ concentration in urine is 25-125 mmol/day, and typical pH range is 4.5-8.0⁶⁹). The results are presented in the Table 2 (below). The difference between the spiked potassium concentration and detected by K⁺ ISE was 13% or less. The pH ISE detected higher levels of pH than a commercial pH meter; difference was 0.3-0.4 pH values. This shift is probably explained by influence of high ionic content of artificial urine on the sensor's work. However, for medical purposes the accuracy of pH detection can be considered sufficient, since the urine pH is highly variable and in case of diseases it can be shifted to 1 pH value or even more. Furthermore, accuracy of the pH and K⁺ detection probably will be better in less complex samples or in diluted urine.

TABLE 2
Results of the detection of K⁺ concentration
and pH level in artificial urine using LIG ISEs

| | Added K⁺ | Detected K⁺ | | | | |
|---|---|---|---|---|---|---|
| | concentration, | concentration, | Recovery, | pH, measured | pH, measured | Recovery, |
| # | mM | mM | % | by pH meter | by pH ISE | % |
| 1 | 10.0 | 11.3 | 113 | 6.71 | 7.08 | 106 |
| 2 | 25.0 | 22.6 | 90 | 7.10 | 7.44 | 105 |
| 3 | 50.0 | 47.0 | 94 | 6.11 | 6.53 | 107 |
| 4 | 125.0 | 108.9 | 87 | 6.51 | 6.97 | 107 |
| 5 | 10.0 | 9.8 | 98 | 7.10 | 7.40 | 104 |

### HRP Sensors for Atrazine Sensing

Amperometric sensing of the herbicide atrazine on LIG platform was demonstrated. Graphene sensors perform well in terms of enzymatic-based sensing due do the direct electron communication between the electrode and active center of the enzyme.³³ Horseradish peroxidase was selected for this enzymatic biosensor due to its high stability and availability⁷⁰. Our procedures involve the initial detection of hydrogen peroxide. HRP has shown selective measurements of hydrogen peroxide through amperometry⁷¹. HRP is a heme group enzyme where the Fe(III) of this group acts as the electron source³⁴. Peroxidases are shown to decompose hydrogen peroxide (H₂O₂) into water and oxygen³⁴. HRP has been used similarly in detecting the herbicide glyphosate at low concentrations³⁷. Therefore, it is reasonable to use HRP in detecting our analyte in PBS pH 7.4.

The atrazine calibration curve in FIG. 19A shows the response change to 10 μM additions of atrazine followed by a PBS addition of the same volume. The detection mechanism utilized hydrogen peroxide as the substrate. Once a signal was generated due to the decomposition of H₂O₂, an herbicide would be added in 10 μM increments. It is evident that HRP shows a higher affinity toward atrazine over the other herbicides tested. Using 3-sigma limit of detection, the theoretical limit of detection for this sensor is 1.6 μM (FIG. 19B). A variety of common herbicides were tested for interference and enzyme selectivity. Of these herbicides, glyphosate, dicamba, and 2,4-dichlorophenoxyacetic acid were tested. After each run, a different herbicide would be tested on the same sensor. The interferant data is shown in FIG. 19C.

### LIG-Pt/LIG-NiO Electrode for Water Splitting

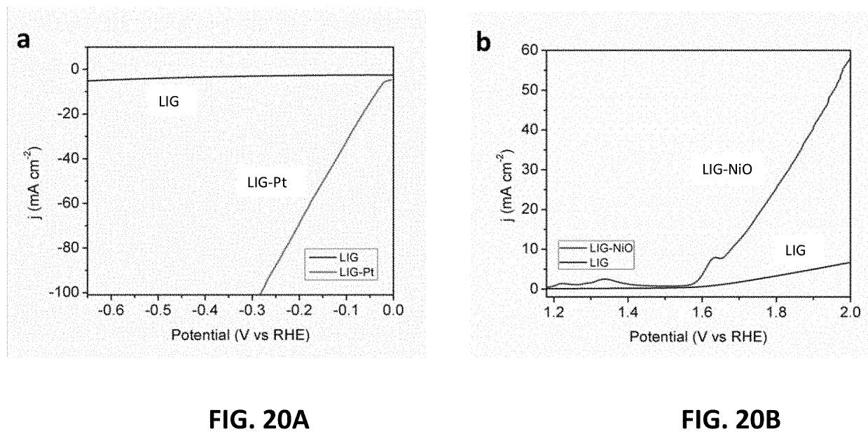

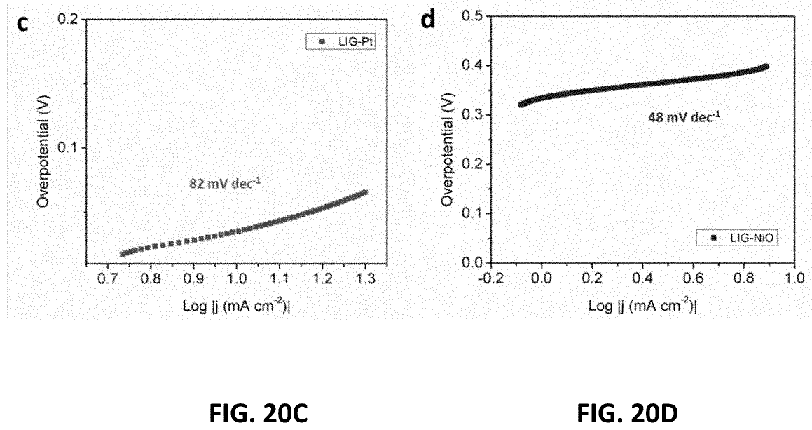

The HER electrode was prepared by electroless deposition of nanoscale amount of Pt onto the LIG. The OER electrode was prepared by thermally decomposit the nickel (II) acetate tetrahydrate by photonic heating. The electrocatalytic activities of LIG-Pt and LAG-NiO toward HER and OER in 1M KOH were measured. Linear-sweep voltammograms (LSV) was performed for OER from 0 V to −1.7 V at a 5 mV/s scan rate, and for OER, from 0 V to 1.7 V at 5 mV/s, respectively. The catalytic activity of the LIG-Pt and LIG-NiO are plotted in FIGS. 20A-B. Analysis of this data includes the conversion of the voltage potential in Ag/AgCl to overpotential in volts vs RHE. The LIG-Pt shows nearly zero onset potential vs RHE, which demonstrated the excellent activity. Overpotentials for current densities of 10 mA/cm² and 100 mA/cm² are commonly used to compare the efficiency of various systems. The OER tests yielded overpotentials of 448 mV and 995 mV for 10 mA/cm² and 100 mA/cm², respectively. The HER tests yielded 35 my and 281 mV for the corresponding current densities. However, the bare LIG (black curve in FIGS. 20A-B) does not show significant current increase in both HER and OER case. Another source of comparison is the Tafel slope. The respective OER and HER Tafel slopes were 48 mV/dec and 82 mV/dec as shown in FIGS. 20C-D. The Tafel slope of LIG-Pt is slightly higher than Pt-based electrode prepared by conventional methods. However, the straightforward electroless deposition method presented here is noteworthy, as it eliminates the needs to prepare inks that containing noble metal power, conductive filler, and binder. The Tafel slope of LIG-NiO is as low as 48 mV/dec, indicating the rapid reaction kinetics on the electrode. This values is similar to recently reported values″. Hence, LIG with liquid phase deposited metal nanoparticle is an important addition to metal based electrocatalysts.

Supporting Information for Water Splitting

With particular reference to FIGS. 25A-F, 26AB, 27A-B, and 28A-D, additional information supporting water splitting electrodes according to an example of the invention includes the following.

Preparation of Water Splitting Electrodes

Preparation of LIG-NiO electrodes. Once LIG electrodes were lasered, 15 of 1M nickel (II) acetate tetrahydrate was deposited onto each electrode. The solution was allowed to dry for 10 minutes. The CO₂ laser, running the same etching pattern, lasered the LIG a second cycle to induce the thermal decomposition of the nickel salt into nickel (II) oxide and other gases, such as oxygenates, hydrogen with hydrocarbons, and carbon oxides. The silver paste was applied to the stem of the LIG electrode. Acrylic polish was applied to the electrode to ensure a constant, working surface area of 25 mm² throughout experiments.

Preparation of LIG-Pt electrodes. The deposition of Pt on LIG is followed by the recipe reported previously (49-52). A chloroplatinic/formic acid solution with a 100:5 ratio, respectively, was used to electrolessly deposit Pt onto bare LIG electrodes. Bare electrodes were placed into a glass vial containing 10 mL of the acid solution. The pH of the solution was changed from 1.68 to 1.75 by adding Ammonium Hydroxide in order to increase the rate of Pt deposition. The electrodes sat in the solution for 24 hours under ambient conditions. In a similar manner to the LIG-NO electrode, silver paste and acrylic polish were applied. See, e.g., FIGS. 25A-F, 26A-B, 27A-B, and 28A-D,

### Conclusions

In conclusion, we report the fabrication of electrochemical ion sensors, pesticide biosensors, and water splitting electrodes by utilizing laser-induced graphene (LIG) platform. The ISE sensors demonstrated wide linear ranges (0.3-150 mM K⁺ and pH 5-8), near-Nernstian sensitivity (53.0 mV/dec for K⁺ ISE and −56.6 mV/pH for pH ISE), and good dry-storage stability (100% of the initial sensitivity after 40 days for K⁺ ISE and 97% after 44 days for pH ISE). These analytical characteristics of the sensors are comparable or exceed previous reports for nanomaterial-based K^{+ 64} and pH^{66,68} solid-state sensors. Practical applicability of the proposed ISEs was confirmed by the analysis of artificial urine that contained 11 metabolites and inorganic ions. The characteristics of the sensors make them suitable for various biological applications, such as detection of K⁺ and pH levels in blood plasma^{73,74}, urine⁷⁵, and other biological fluids^{76,77}.

Simple and efficient LIG electrodes were developed for potentiometric sensing and water splitting. The HRP pesticide biosensors created by functionalizing the LIG electrodes permitted the selective monitoring of the herbicide atrazine with a high sensitivity (28.9 mA/μM) with negligible inference from other commons herbicides (glyphosate, dicamba, and 2,4-Dichlorophenoxyacetic acid). The LIG electrodes also exhibit small overpotential, 35 mV for hydrogen evolution reaction and 448 my for oxygen evolution reaction at 10 mA/cm² after platinum and nickel oxide are immobilized. LIG can be used as electrodes directly after lasing, without necessity in any further treatments that are required in case of printed graphene electrodes⁵³. The fabrication procedure is easily scalable for mass production of the electrodes. Such low cost, fast and simple one-step procedure for the electrode preparation also eliminates the need in modification of the electrode with nanomaterials as was necessary in earlier works^{21,64,66}. In summary, the presented LIG electrodes can serve as a versatile platform for the creation of solid-state ISEs, enzymatic electrochemical sensing, and water splitting applications. Additionally, laser induction of polyamide as a fast process could fabricate electrodes on a large scale. The solution phase deposition of metal precursor could also be integrated into roll-to-roll production of such electrodes, which is amenable to potential scale-up and commercialization.

### References (for Specific Examples 2-4 Section)

1 L. Tang, Y. Wang, Y. Li, H. Feng, J. Lu and J. Li, Adv. Puna. Mater., 2009, 19, 2782-2789.
2 R. Yan, S. Qiu, L. Tong and Y. Qian, Chem. Speciat. Bloavailab., 2016, 28, 72-77.
3 J. Albero, D. Mateo and H. Garcia, Molecules, 2019, 24, 906.
4 Y. Zhang, L. Zhang and C. Zhou, Acc. Chem. Res., 2013, 46, 2329-2339.
5 S. Unarunotai, Y. Murata, C. E. Chialvo, H. Kim, S. MacLaren, N. Mason, 1. Petrov and J. A. Rogers, Appl. Phys. Lett., 2009, 95, 202101.
6 S. Bae, H. Kim, Y. Lee, X. Xu, J.-S. Park, Y. Zheng, J. Balakrishnan, T. Lei, H. Ri Kim, Y. Il Song, Y.-J. Kim, K. S. Kim, B. Özyilmaz, J.-H. Ahn, B. H. Hong and S. Iijima, Nat. Nanotechnol., 2010, 5, 574-578.
7 S. R. Das, Q. Nian, A. A. Cargill, J. A. Hondred, S. Ding, M. Saei, G. J. Cheng and J. C. Claussen, Nanoscale, 2016, 8, 15870-15879. [incorporated by reference herein].
8 A. Moya, G. Gabriel, R. Villa and F. Javier del Campo, Curr. Opin. Electrochem., 2017, 3, 29-39.
9 X. Huang, T. Leng, M. Zhu, X. Zhang, J. Chen, K. Chang, M. Ageeli A. K. Geim, K. S. Novoselov and Z. Hu, Sei. Rep., 2016, 5, 18298.
10 K. Fu, Y. Y. Wang, C. Yan, Y. Yao, Y. Chen, J. Dai, S. Lacey, Y. Y. Wang, J. Wan, T. Li, Z. Wang, Y. Xu and L. Hu, Adv. Mater., 2016, 28, 2587-2594.
11 S. R. Das, S. Srinivasan, L. R. Stromberg, Q. He, N. Garland, W. E. Straszheim, P. M. Ajayan, G. Balasubramanian and J. C. Claussen, Nanoscale, DOI:10.1039/c7nr06213c.
12 E. B. Secor, P. L. Prabhumirashi, K. Puntambekar, M. L. Geier and M. C. Hersam, Phys. Chem. Lett., 2013, 4, 1347-1351.
13 E. B. Secor, B. Y. Ahn, T. Z. Gao, J. A. Lewis and M. C. Hersam, Adv. Mater., 2015, 27, 6683-6688. [incorporated by reference herein].
14 J. Lin, Z. Peng, Y. Liu, F. Ruiz-Zepeda, R. Ye, E. L. Ci Samuel, M. J. Yacaman, B. I. Yakobson and J. M. Tour, Nat. Commun., DOI:10.1038/ncomms6714.
15 R. Ye, D. K. James and J. M. Tour, Acc. Chem. Res., 2018, 51, 1609-1620. [incorporated by reference herein].
16 D. Vanegas, L. Patifio, C. Mendez, D. Oliveira, A. Torres, C. Gomes and E. McLamore, Biosensors, 2018, 8, 42. [incorporated by reference herein].
17 C. Fenzl, P. Kayak, T. Hirsch, 0. S. Wolfbeis, H. N. Alshareef and A. J. Baeumner, ACS Sensors, 2017, 2, 616-620.
18 J. Zhang, C. Zhang, J. Sha, H. Fei, Y. Li and J. M. Tour, ACS Appl. Mater. Interfaces, 2017, 9, 26840-26847. [incorporated by reference herein].
19 J. Zhang, M. Ren, Y. Li and J. M. Tour, ACS Energy Lett., 2018, 3, 677-683. [incorporated by reference herein].
20 N. T. Garland, E. S. McLamore, N. D. Cavallaro, D. Mendivelso-Perez, E. A. Smith, D. Jing and J. C. Claussen, ACS Appl. Mater. Interfaces, 2018, 10, 39124-39133. [incorporated by reference herein].
21 Q. An, S. Gan, J. Xu, Y. Bao, T. Wu, H. Kong, L. Zhong Y. Ma, Z. Song and L. Niu, Electrochem. commun., 2019, 107, 106553.
22 R. R. A. Soares, R. G. II ort, C. C. Pola, K. Parate, E. L. Reis, N. F. F. Soares, E. S. McLamore, J. C. Claussen and C. L. Gomes, ACS Sensors, DOI:10.1021/acssensors.9b02345. [incorporated by reference herein].
23 P. Puetz, A. Behrent, A. Baeumner and J. Wegener, Sensors Actuators B Chem., 2020, 321, 128443.
24 Y. Yang, Y. Song, X. Bo, J. Min, 0. S. Pak, L. Zhu, M. Wang, J. Tu, A. Kogan, H. Zhang, T. K. Hsiai, Z. Li and W. Gao, Nat. Biotechnol., 2020, 38, 217-224.
25 Y. Kim and S. Amemiya, Anal. Chem., 2008, 80, 6056-6065.
26 A. Malon, A. Radu, W. Qin, Y. Qin, A. Ceresa, M. Maj-Zurawska, E. Bakker and E. Pretsch, Anal. Chem., 2003, 75, 3865-3871.
27 I. S. Kucherenko, D. Sanborn, B. Chen, N. Garland, M. Serhan, E. Forzani, C. Gomes and J. C. Claussen, Adv. Mater. Technol., DOI:10.1.002/adint.201901037. [incorporated by reference herein].
28 J. Hu, A. Stein and P. Bühlmann, TrAC—Trends Anal. Chem., 2016, 76, 102-114.
29 M. Novell, T. Guinovart P. Blondeau, F. X. Pius and F. J. Andrade, Lab Chip, 2014, 14, 1308-1314.
30 E. Lindner and R. E. Gyurcsányi, J. Solid State Electrochem., 2009, 13, 51-68.
31 C. Zuliani and D. Diamond, Electrochim. Acta, 2012.
32 G. Dimeski, T. Badrick and A. S. John, Clin. Chim. Acta, 2010, 411, 309-317.
33 Y. Shao, J. Wang, H. Wu, J. Liu, I. A. Aksay and Y. Lin, Electroanalysis, 2010, 22, 1027-1036.
34 Z. Tao, R. A. Raffel, A.-K. Souid and J. Goodisman, DOI:10.1016/j.bpj.2008.11.071.
35 B. Bucur F. D. Munteanu, J. L. Marty and A. Vasilescu, Biosensors, 2018, 8, 27.
36 R. W. Keay and C. J. McNeil, Biosens. Bioelectron., 1998, 13, 963-970.
37 E. A. Songa, a A. Arotiba, J. H. O. Owino, N. Jahed, P. G. L. Baker and E. I. Iwuoha, Bioelectrochemistry, 2009, 75, 117-123.
38 I. Roger, M. A. Shipman and M. D. Symes, Nat. Rev. Chem., 2017, 1, 0003.
39 J. Wang, W. Cui, Q. Liu, Z. Xing, A. M. Asiri and X. Sun, Adv. Mater., 2016, 28, 215-230.
40 S. Anantharaj, S. R. Ede, K. Sakthikumar, K. Karthick, S. Mishra and S. Kundu, ACS Catal., 2016, 6, 8069-8097.
41 J. A. Buss, M. Hirahara, Y. Ueda and T. Agapie, Angew. Chemie—Int. Ed., 2018, 57, 16329-16333.
42 Y. Tan, H. Wang, P. Liu, Y. Shen, C. Cheng, A. Hirata, T. Fujita, Z. Tang and M. Chen, Energy Environ. Sci., 2016, 9, 2257-2261.
43 L. Trotochaud, S. L. Young, J. K. Ranney and S. W. Boettcher, J. Am. Chem. Soc., 2014, 136, 6744-6753.
44 B. Zhang, Y. H. Lui, A. P. S. Gaur, B. Chen, X. Tang, Z. Qi and S. Hu, ACS Appl. Mater. Interfaces, 2018, 10, 8739-8748.
45 B. Zhang, Z. Qi, Z. Wu, Y. H. Lui, T. H. Kim, X. Tang, L. Zhou, W. Huang and S. Hu, ACS Energy Lett., 2019, 4, 328-336.
46 L. L. Feng, M. Fan, Y. Wu, Y. Liu, G. D. Li, H. Chen, W. Chen, D. Wang and X. Zou, J. Mater, Chem. A, 2016, 4, 6860-6867.
47 X. Song, H. Zhao, K. Fang, Y. Lou, Z. Liu, C. Liu, Z. Ren, X. Zhou, H. Fang and Y. Zhu, RSC Adv., 2019, 9, 3113-3119.
48 P. Nayak, Q. Jiang, N. Kurra, X. Wang, U. Buttner and H. N. Alshareef, J. Mater. Chem. A, 2017, 5, 20422-20427.
49 J. C. Claussen, M. A. Daniele, J. Geder, M. Pruessner, A. J. Makinen, B. J. Meide, M. Twigg, J. M. Verbarg and I. L. Medintz, ACS Appl. Mater. Interfaces, 2014, 6, 17837-17847.
50 K. M. Marr, B. Chen, E. J. Mootz, J. Geder, M. Pruessner, B. J. Melde, R. R. Vanfleet, I. L. Medintz, B. D. Iverson and J. C. Claussen, ACS Nano, 2015, 9, 7791-7803. [incorporated by reference herein].
51 B. Chen, N. T. Garland, J. Geder, M. Pruessner, E. Mootz, A. Cargill, A. Letters, G. Vokshi, J. Davis, W. Burns, M. A. Danielle, J. Kogot, I. L. Medintz and J. C. Claussen, ACS Appl. Mater. Interfaces, 2016, 8, 30941-30947. [incorporated by reference herein].
52 B. Chen, A. Gsalla, A. Gaur, Y. H. Lui, X. Tang, J. Geder M. Pruessner, B. J. Melde I. L. Medintz, B. Shafei, S. Hu and J. C. Claussen, ACS Appl. Nano Mater., 2019, 2, 4143-4149. [incorporated by reference herein].
53 Q. He, S. R. Das, N. T. Garland, D. Jing, J. A. Hondred, A. A. Cargill, S. Ding, C. Karunakaran and J. C. Claussen, ACS Appl. Mater. Interfaces, 2017, 9, 12719-12727. [incorporated by reference herein].
54 J. Zhang, Y. Guo, S. Li and H. Xu, Meas. Sci. Thehnol., 2016, 27, 105101.
55 T. Brooks and C. W. Keevil, Lett. Appl. Microbiol., 1997, 24, 203-206.
56 A. C. Ferrari, J. C. Meyer, V. Scardaci, C. Casiraghi, M. Lazzeri, F. Mauri, S. Piscanec, D. Jiang, K. S. Novoselov, S. Roth and A. K. Geim, Phys. Rev. Lett., 2006, 97, 187401.
57 D. A. C. Brownson, G. C. Smith and C. E. Banks, R. Soc. Open Sci., 2017, 4, 171128.
58 J. Liu, L. Zhang, C. Yang and S. Tao, J. Mater. Chem. A, 2019, 7, 21168-21175.
59 J. C. Claussen, M. S. Artiles, E. S. McLamore, S. Mohanty, J. Shi, J. L. Rickus, T. S. Fisher and D. M. Porterfield, J. Mater. Chem., 2011, 21, 11224.
60 J. Li, A. Cassell, L. Delzeit, J. Han and M. Meyyappan, J. Phys. Chem. B, 2002, 106, 9299-9305.
61 R. R. Moore, C. E. Banks and R. G. Compton, Anal. Chem., 2004, 76, 2677-2682.
62 J. H. Kim, J.-Y. Hwang, H. R. Hwang, H. S. Kim, J. H. Lee, J.-W. Seo, U. S. Shin and S.-H. Lee, Sci. Rep., 2018, 8, 1375.
63 D. H. Youn, J. S. J.-W. Jang, J. Y. Kim, J. S. J.-W. Jang, S. H. Choi and J. S. Lee, Sci. Rep., 2015, 4, 5492.
64 F. Li, J. Ye, M. Zhou, S. Gan, Q. Zhang, D. Han and L. Niu, Analyst, 2012, 137, 618-623.
65 M. Fouskaki and N. Chaniotakis, Analyst, 2008, 133, 1072-1075.
66 W. W. A. Wan Salim, A. C. Hermann, M. A. Zietchek, J. E. Pfluger, J. H. Park, A. ul Hague, F. Sanober and D. M. Porterfield, in International Conference for Innovation in Biomedical Engineering and Life Sciences, 2016, pp. 86-93.
67 W. S. Han, K. C. Chung, M.-H. Kim, H.-B. Ko, Y. H. Lee and T.-K. Hong, Anal. Sci., 2004, 20, 1419-1422.
68 M.-H. Piao, J.-H. Yoon, G. Jeon and Y.-B. Shim, Sensors, 2003, 3, 192-201.
69 A. Lewenstam, Electroanalysis, 2014, 26, 1171-1181.
70 A. L. Simonian, T. A. Good, S. S. Wang and J. R. Wild, Anal. Chim. Acta, 2005, 534, 69-77.
71 J. Wang, B. Freiha, N. Naser, E. Gonzalez Romero, U. Wollenberger, M. Ozsoz and O. Evans, Anal. Chico. Acta, 1991, 254, 81-88.
72 Structure, function and operational stability of peroxidases.
73 E. Kardalas, S. A. Paschou, P. Anagnostis, G. Muscogiuri, G. Siasos and A. Vryonidou, Endocr. Connect., 2018, 7, R135-R146.
74 R. Carnauba, A. Baptistella, V. Paschoal and G. Hübscher, Nutrients, 2017, 9, 538.
75 A. K. Leonberg-Yoo, H. Tighiouart, A. S. Levey, G. J. Beck and M. J. Sarnak, Am. J. Kidney Dis., 2017, 69, 341-349.
76 U. Udensi and P. Tchounwou, Int. Clin. Exp. Physiol., 2017, 4, 111.
77 W. Aoi and Y. Marunaka, Biomed Res. Int., 2014, 2014, 1-8.

### 3 Specific Embodiment and Example 5

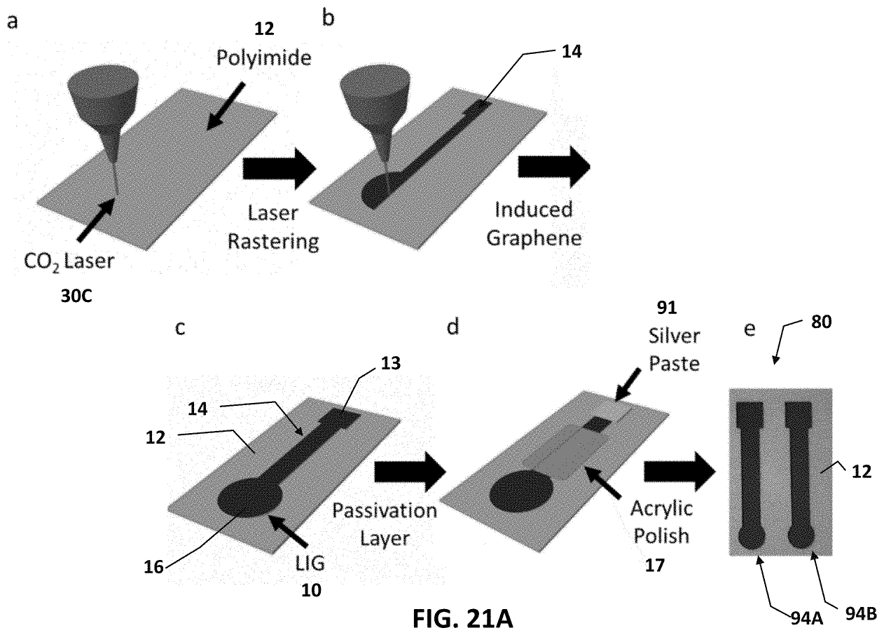

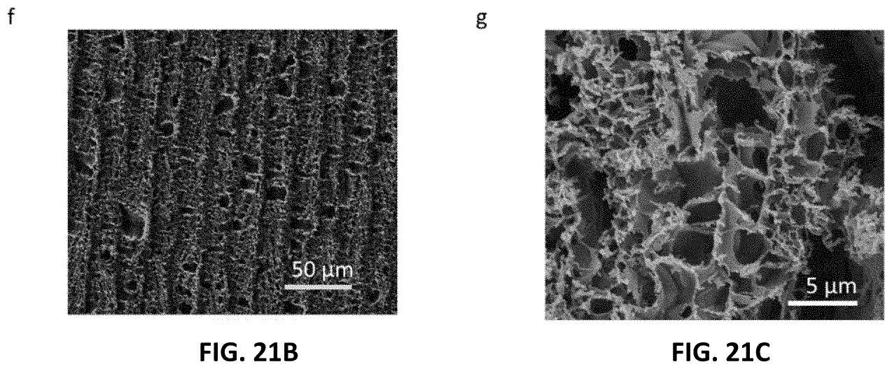

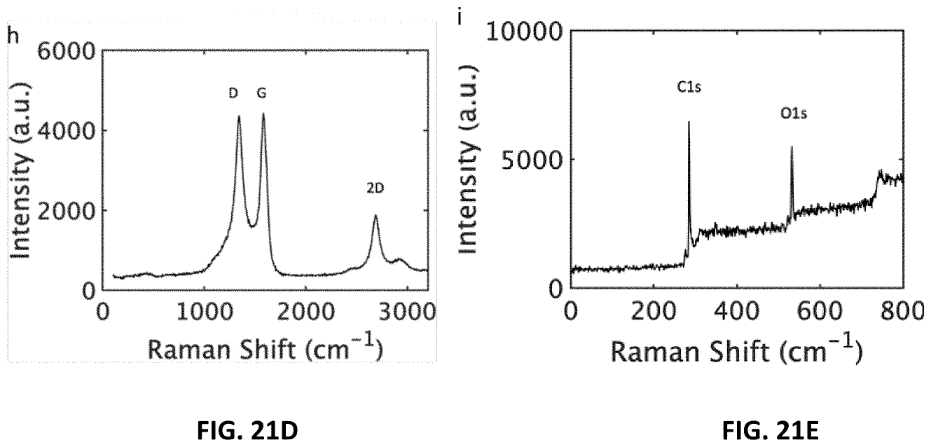

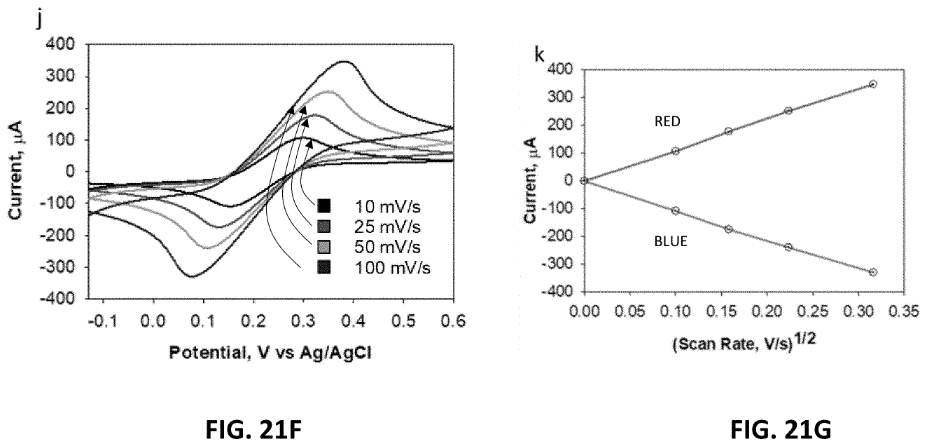

With particular reference to FIGS. 21 to 24A-B, specific embodiments of apparatus, methods, and systems according to the invention are set forth in more detail. In particular, these examples utilize LIG electrodes like those in Specific Example 1 and Specific Examples 2-4, supra, but configured for different applications, as discussed below.

### Biorecognition Element-Free Electrochemical Sensing of Neonicotinoids Using Laser-Induced Graphene

### Abstract

Growing food demand requires increasing agricultural yield, which is ensured largely in part by the application of pesticides. However, the over application and migration of pesticides can affect vital insect populations and harm the environment. Current methods to measure pesticide levels and prevent over applications are time consuming, complex, and not economical. As such, there is a need to develop a time-efficient, economical sensor for the accurate detection of pesticides. One specific group of pesticides, neonicotinoids, has been shown to be specifically toxic toward honey bees and environmental biodiversity. As such, four different kinds of neonicotinoids were analyzed specifically in this work: clothianidin, imidacloprid, thiamethoxam, and dinotefuran. A laser induced graphene (LIG) device was developed and used in a standard three electrode electrochemical cell setup. The sensor showed a linear range of 10-40 μM and detection limit of 823, 384, 338, and 682 nM for clothianidin, imidacloprid, thiamethoxam, and dinotefuran, respectively. These results indicate that LIG sensors can be utilized for the selective detection of neonicotinoids without the use of a biorecognition agent.

### Introduction

The development of neonicotinoid insecticides over three decades ago is considered a major success in agrochemical research due to their ability to eliminate a broad spectrum of sucking and certain chewing pests that can damage croplands, home yards and gardens, and golf course greens.¹ Neonicotinoids exhibit physicochemical advantages over previous generations of pesticides for pest management that have led them to become the fastest growing insecticide used; neonicotinoids account for over 25% of the global pesticide market.² Neonicotinoids act within target pests by interfering with the acetylcholine neurotransmitter ultimately causing neurotoxicity and they operate systemically by dispersing throughout a plant system which consequently causes the plant to become toxic to specific herbivores.³ Hence, neonicotinoids have negatively impacted a large number of non-target organisms including a variety of aquatic species⁴ and honeybees⁵ primarily due to the significant down-regulation of gene expression related to metabolism and detoxification⁷ More specifically, imidacloprid (DAD), thiamethoxam (TMX), clothianidin (CLT), and dinotefuran (DNT) (including their variants S-dinotefuran and R-dinotefuran) are the most widely used neonicotinoids and they have been shown to be toxic to non-target species such as honeybees with acute oral LD₅₀ ranging from (0.004-0.005) μg per bee.^{9 8, 10} Hence these four major neonicotinoids have started to diminish the biodiversity of the ecosystems in which they are used.⁶

The role of neonicotinoids causing human disease (e.g., cancer) and their long-term effects on ecosystems cannot be conclusively pinpointed due to poor neonicotinoid exposure assessment.²².²² Neonicotinoids are becoming more prevalent in a variety of ecosystems especially in rural agricultural settings as neonicotinoids present in high concentrations in seed dressings can drift to neighboring areas during the seed sowing process and only 1.6-20% of the active neonicotinoid ingredient in the seed dressing is absorbed by the plant, leaving the majority in the soil. Hence these agrochemicals have percolated into watersheds and even drinking waters in the agricultural regions of the Midwestern United States (i.e., in the state of Wisconsin) including in private portable wells at high concentrations (TMX: 1.43 μg/L, IMD: 1.59 μg/L, CLT: 3.88 μg/L). Perhaps more concerning is a recent study that found neonicotinoids in treated municipal drinking water in the state of Iowa (another state of the Midwestern United States) which suggests that conventional water treatment practices are unable to eliminate neonicotinoids from the water supply. Hence wide-scale knowledge and mapping of neonicotinoid drift, runoff, exposure, and endpoints is critically important information to help oversee appropriate neonicotinoid stewardship and application methodologies in order to minimize their environmental impact.

Currently, there are no commercially available in-field sensors available for wide-scale neonicotinoid testing. The standard method for quantifying concentrations of pesticides from field samples requires packaging the contaminated samples of soil or water and shipping them back to a laboratory for analysis using gas or liquid chromatography coupled with mass spectrometry.²⁵ These methods are accurate and reproducible for identifying and quantifying analytes, but are time-consuming and expensive. They require extensive sample preparation, highly trained personnel and specialized instrumentation. Inherently, these methods cannot provide real-time or in-field analyte concentrations nor be used to screen large sample sets in a timely and cost-effective manner. Commercially available pesticide field sensors are few and generally consist of colorimetric test strip papers (e.g., Pesticide Detection Test Cards by RenekaBio, Agri-Screen®, Tickets by Neogen, and OrganaDx by MyDx) that are limited to qualitative (high/negative) outputs, limited to testing organophosphates and carbamates, and easily fouled/misread due to particulate matter found in fieldsamples.³⁰

Electrochemical-based biosensors offer a promising solution to monitoring neonicotinoid concentrations in a rapid, quantifiable, low-cost manner that is suitable for large-scale use. Electrochemical biosensors are widely researched for in-field sensing as they are not affected by optically dense, turbid fluid that can negatively affect colorimetric sensors and they provide an electrical signal that can be directly correlated to analyte concentration levels.³² Various reports of electrochemical neonicotinoid sensors exist in the literature using a variety of biorecognition agents including molecular imprinted polymers, aptamers, antibodies, quenchbodies, and (Q-bodies), with favorable results for monitoring TMX, IMD, DNT, and/or CLT down to detection limits of indicated in those publications. The limitation of these sensors of course is that they only can be used once (antibody/aptamer binding to target molecules generally cannot be undone within the context of in-field test conditions), suffer from limited shelf-life due to biorecognition agent degradation, have been limited to monitoring just one or more neonicotinoids. To create biorecognition element-free neonicotinoid sensors, researchers have used a variety of materials including bismuth, β-cyclodextrin (β-CD), silver, and metal-organic frameworks (MOFs) to directly reduce/oxide the pesticides to create a measurable electrochemical signal. However, the construction of such sensors is tedious requiring sputter coating, hydrothermal nanoparticle synthesis, nanoparticle chemical coprecipitation synthesis, magnetic separation, centrifugation, solvent soaking/rinsing, and results are poor. Some work has even attempted to monitoring all four of these four neonicotinoids. Perhaps some of the more promising results in terms of sensor sensitivity and longevity, have come with sensors based on carbon nanomaterials. A variety of carbon-based nanomaterials have been used for neonicotinoids sensing including graphene oxide, nanoparticle decorated carbon composites, graphene and graphene oxide modified glassy carbon electrode, macro-meso-microporous carbon composite. However, these sensors are not produced in a scalable fashion as they require multiple chemical processing steps including centrifugation, solvent soaks, washing, stirring/mixing, high temperature pyrolysis (800° C.), heated/fuming acid etching, and electrode polishing to create the carbon nanomaterials and to cast them onto electrode surfaces. Moreover these sensors are not able to be used continuously or have low shelf-life due to the use of biorecognition agents, and/or have not shown to be capable of measuring signal from all four major neonicotinoids, viz., TMX, IMD, DNT, CLT.

Perhaps one of the more scalable routes to creating carbon nanomaterial-based electrochemical sensors has been with the use of laser-scribed graphene often referred to as laser induced graphene (LIG).¹² LIG is created through a direct-write process that circumvents the need to create graphene/graphene oxide via chemical synthesis (processes that often include high temperature pyrolysis and/or high temperature acid etching as used in the Hummer's or Brodie method) or create graphene through high-temperature chemical vapor. LIG also eliminates the need for chemical synthesis of graphene and the subsequent ink formulation associated with more scalable printed graphene electrode processes (e.g., screen, gravure, inkjet, and aerosol printing) as well as eliminates the need for mask, stencils, and pattern rolls associated with screen and gravure printing for example. Moreover, LIG eliminates the need for post-print annealing (e.g., rapid-pulse, thermal, or photonic annealing) that is needed to evaporate ink solvent and carbonize ink binders (e.g., ethyl and nitro cellulose) to make the printed graphene sufficiently conductive for electronic applications such as sensing. The multi-layered, turbostratic structure of LIG improves the physical properties (more resistant to shear stress) of the graphene as compared to conventional AB-stacked (Bernal) structured graphene. Furthermore, the porous and high-defect nature of LIG improves heterogenous charge transport during electrochemical sensing which consequently improves target analyte to electrode mass transport/binding, reduces the ohmic reduction in electrical potential, and leads to the ability to measure small currents within low sample volumes.¹⁵ Hence researchers have used LIG to electrochemically sense a wide variety of target analytes including sensing fertilizer and hydrations ions in soil and sweat respectively^{20,21}, monitoring of biogenic amines and Salmonella bacteria in food samples^{16,22}, impedance-based cell monitoring²³, aptamer-based biosensing in serum¹⁷, and biomarkers in sweat²⁴. To our knowledge, only one example exists of using LIG for pesticide detection exists in which the enzyme organophosphorus hydrolase was immobilized on the graphene electrode detection of the organophosphate methyl parathion on actual crop surfaces in real time.¹⁹ These results are promising but the irreversible inhibition of the enzyme does not permit multiple uses and its shelf-life would be limited to the use of a biorecognition element. There does not exist in the research literature to date a LIG sensor capable of neonicotinoid monitoring.

Herein, we report the first example of using LIG for neonicotinoid sensing. We demonstrate how the biorecognition element-free LIG electrode without the use of biorecognition agents can be used to selectively monitor the four major neonicotinoids (CLT, IMD, TMX, and DNT) with a rapid response time (˜10 s) through the use of square voltammetry. More specifically the sensor is capable of monitoring these major neonicotinoids from other pesticides that are commonly used in conjunction with neonicotinoids in the Midwestern United States including other broad-leaf insecticides such as parathion, paraoxon, and fipronil as well as systemic herbicides such as glyphosate (Roundup®) atrazine, dicamba, and 2,4-dichlorophenoxyacetic acid (2,4-D). The reported sensor obtained low detection limits (CLT—823 nM; IMD—384; TMX—338; and DNT 682 nM) which are below the regulatory threshold level (xx nM) in fresh water.²⁷ The corresponding limits of detection for. Finally, neonicotinoid monitoring was confirmed in actual complex biological matrices by river water samples collected from the South Skunk River in Iowa.

### Results and Discussion

### LIG Electrode Fabrication

The fabrication of LIG consists of preparing the polyimide (PI) substrate 12, positioning the laser 30, laser rastering which induces the graphene 14, and the application of acrylic polish 17 and silver ink 91 (FIG. 21A, steps a-e). The PI substrate 12 is first washed with isopropyl alcohol and the laser 30 is positioned over the LIG 10 with a 2 mm defocus. The settings used for the laser rastering and the development of LIG 10 are as follows: 7% power, 15% speed, and 50% frequency. These parameters were used to induce a 5 mm diameter dipstick design 14. Following the creation of the LIG 10, acrylic polish 17 was added to prevent wicking and maintain a constant working area on the electrode 10. Silver ink 91 was added to the contact to prevent abrasion from the potentiostat clips.

### LIG Electrode Characterization

The graphene morphology and thickness were characterized using scanning electron microscopy (SEM, FIGS. 21B and C). The low magnification image shows a groove-like morphology along the lasing direction while high magnification image shows numerous pores at different scales.

The chemical compositions of the obtained LIG were analyzed by X-Rays photoelectron spectroscopy (XPS) (FIG. 21E). Strong XPS signal was obtained from the carbon (C) C1s peak and the oxygen (O) O1s peak which resulted in a C/O ratio of 9.1, which indicates a high degree of carbonization of the PI after lasing. Raman spectroscopy confirmed the presence of multilayered graphene after laser scribing the PI 12 as the 2D/G peak ratio of the carbon surface was 0.4 (FIG. 21D). More specifically, the D peak was observed at 1342 cm⁻¹, which is characteristic of defects on the graphene surface 14. Similarly, the G peak that is characteristic of the sp² bonded carbon atoms was observed at 1583 cm⁻¹. Meanwhile, the 2D band that is indicative of double resonance electron-phonon scattering in graphene was observed at 2687 cm⁻¹. The electroactive surface area (ESA) was calculated using the Randle-Sevcik equation (Eq. 1) by recording the anodic and cathodic current peak at varying scan rates (FIGS. 21F-G). The equation expresses the oxidation/reduction peak current (ampere) in the following terms: A is the electroactive surface area (cm²), D is the diffusion coefficient (7.6×10⁻⁶ cm²s⁻¹), n is the number of electrons transferred in the redox probe, v is the scan rate (Vs⁻¹), and C is the bulk concentration of the redox species (5 mM).

iₚ=2.69×10⁵AD^{1/2}n^{3/2}v^{1/2}c (1)

The ESA was calculated as 0.313 cm² which is 160% of the geometric surface area (0.196 cm²).

### Electrochemical Sensing of Neonicotinoids

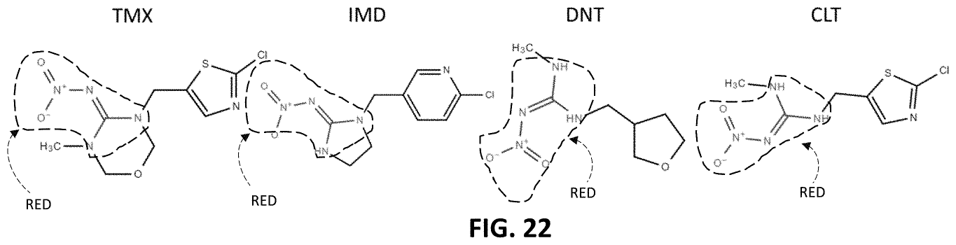

LIG electrodes 80 made by the above-technique (see FIG. 21A) were calibrated for increasing concentrations of thiamethoxam (TMX), dinotefuran (DNT), imidacloprid (IMD), and clothianidin (CLT) using square wave voltammetry. It should be noted here that square-wave voltammetry (SWV) is a particular type of differential pulse voltammetry that applies potential in a stepwise manner while recording the anodic or cathodic current associated with target molecule redox potentials. SWV holds an advantage over cyclic voltammetry and linear sweep voltammetry as SWV amplifies faradaic current and mitigates noise from capacitive current.²⁴ Neonicotinoids possess a nitro group that is reduced to a hydroxylamine group, occurring near −1.00 V vs Hg hanging drop electrode, that are monitored via SWV herein.²⁶ The reduction of the nitroguanidine group of neonicotinoid pesticides is exceedingly complicated due to various protonations, tautomerization of the intermediate species, and possible adduct formation of nitroso and hydroxylamine groups. The redox potential of this functional group is pH-dependent, but cannot be modelled in a linear manner over a wide range. The linear ranges of the two-electron reduction of nitroguanidine to nitrosoguanidine are well-established below a pH of 7 and above a pH of 8, but become murky in between. This is presumably due to a variety of different protonation species. Furthermore, the four-electron reduction of nitrosoguanidine to aminoguanidine has a pH-dependent redox potential which is linearly dependent below a pH of 6, between a pH of 8 and 10, and above a pH of 10. Between a pH of 6 and 8, the reduction of nitrosoguanidine functional groups to aminoguanidine is murky, and is probably a concert of differentially protonated species. Neonicotinoid peaks on average occurred within the potential window of −1.18 to −1.24 V, thus making it difficult to distinguish different neonicotinoids when combined in the same solution but enabled the selective detection of neonicotinoids against other insecticides and other classes of pesticides such as herbicides. This is attributed to the reduction of the same nitroguanidine group present in all of the neonicotinoids. The chemical structure and the nitroguanidine group vital for the reduction signal is shown in FIG. 22.

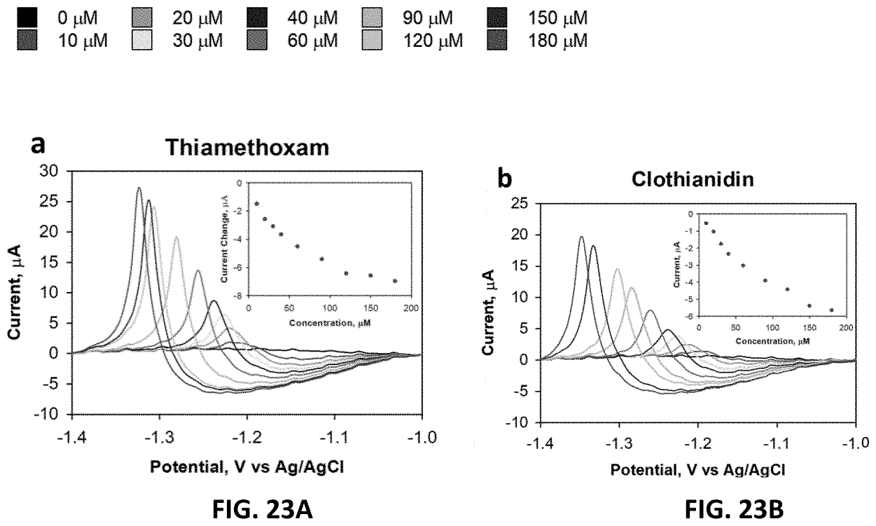

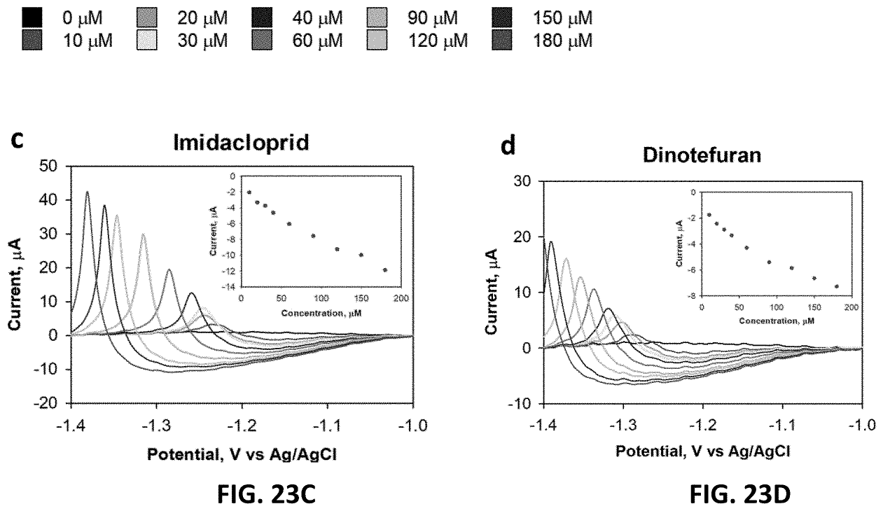

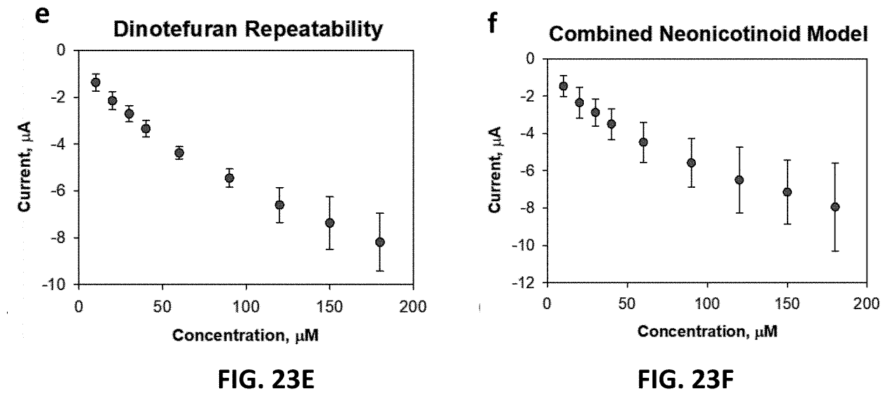

The current reduction peaks increased with increasing concentrations of neonicotinoids (FIG. 23A-D). Calibration repeats were performed n=5 times for DNT with an average relative standard deviation of 13%, highlighting the reproducibility among multiple sensors (FIG. 23E). Overall, the sensor displayed a linear range of 10-40 μM and detection limit of 823, 384, 338, and 682 nM for CLT, IMD, TMX, and DNT respectively. This detection limits were considerably lower than the detection limits (8.3 μM and 7.9 μM for TMX and IMD respectively) reported that also used SWV.²⁵

Next, the electrode response to all four neonicotinoids introduced simultaneously in the same test vial was monitored. To this end an overall neonicotinoid calibration was modeled to predict the concentration of a combined neonicotinoid solution (FIG. 23F). This was done by averaging the calibration plots of each neonicotinoid due to their similar responses. A 40 μM solution of neonicotinoids (10 μM each of TMX, DNT, CLT, IMD) was tested to confirm the accuracy of this model (Table 3, below). On average, this model predicted a concentration of 42.4 μM showing a 106% accuracy. Regarding the anomalous oxidation peak seen with all neonicotinoid insecticides at a pH of 7.4, it may be that some of the species protonate rapidly after formation to give the illusion of an increased current. The disappearance of this oxidation peak at a pH of 8 might be the result of more controlled reduction of the intermediates, slower acid-catalyzed hydrolysis of intermediate adducts, or slower protonation rates. All of these could spread out an oxidation peak such that it becomes overwhelmed by the reduction curve. This does not preclude the possibility of over-reduction of the guanidine portion of the aminoguanidine functional group which results in electron release following a chemical rearrangement.

TABLE 3
Combined Neonicotinoid Found Concentration for 40 μM Spike

| Sensor 1 | Sensor 2 | Sensor 3 | Sensor 4 | Average | Accuracy |
|---|---|---|---|---|---|
| 42.5 μM | 36.1 μM | 48.2 μM | 42.8 μM | 42.4 μM | 106% |

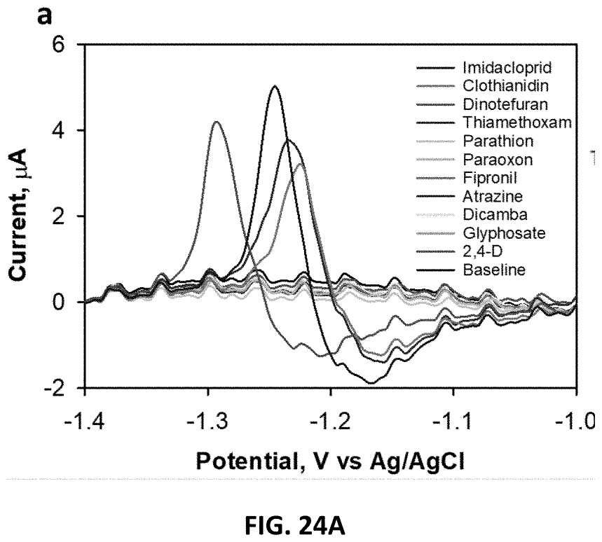

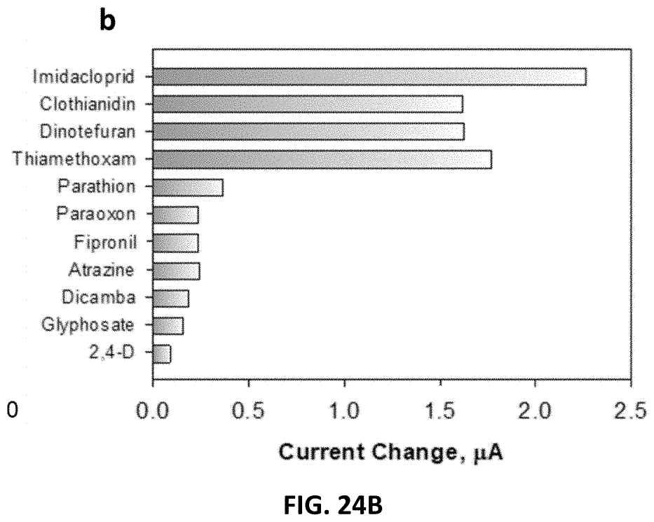

Furthermore, the neonicotinoid reduction potentials were compared with potential interference species. These pesticides include the common herbicides atrazine, glyphosate, dicamba, and 2,4-dichlorophenoxyacetic acid as well as common insecticides fipronil, parathion, and paraoxon (FIGS. 24A and B). Square wave voltammograms were recorded for 40 μM concentrations of each pesticide. None of the potential interference pesticides showed considerable current change within the neonicotinoid reduction potential window of −1.18-1.24 V.

Recovery tests were performed in water from the South Skunk River in Iowa. The water was filtered through a 0.45 μm filter and brought to 0.5 M NaCl. The solution was spiked with 40 μM of a neonicotinoid. For comparison, neonicotinoids were detected in deionized water that was also brought to 0.5 M NaCl. The recovery percentages are presented in Table 4 (below) and were calculated using the river water recording as the found response and the deionized water recording as the standard response.

TABLE 4
River Water Recovery Test

| Neonicotinoid | River Water, μM | DI Water, μM | Accuracy, % |
|---|---|---|---|
| Clothianidin | 56.8 | 49.7 | 114.3 |
| Imidacloprid | 29.3 | 27.9 | 105.0 |
| Thiamethoxam | 46.4 | 29.0 | 160.0 |
| Dinotefuran | 43.5 | 47.1 | 92.4 |

### Conclusion

In conclusion, this work demonstrates one of the first studies to electrochemically determine a family of pesticides, specifically the presence of neonicotinoids, without the use of a biorecognition agent. The reported sensor showcases the first use of LIG as a viable platform for the rapid detection of neonicotinoids and demonstrates a more scalable route to multilayered graphene electrodes than more conventional fabrications methods that use low throughput CVD or chemical/mechanical exfoliation of graphite for graphene synthesis and methods that use screen, gravure, aerosol, inkjet printing to form the electrode. The lack of a biorecognition agent allows for easy use, ease of fabrication, and less stringent storage protocol. Additionally, the detection of neonicotinoids was not affected by other common herbicides and insecticides, thus bolstering the reported platform as highly selective. The sensor accurately detected neonicotinoid concentrations in complex media such as river water, with accuracies of 114.3, 105.0, 160.0, and 92.4% for clothianidin, imidacloprid, thiamethoxam, and dinotefuran, respectively. The sensor showed a linear range of 10-40 μM and detection limit of 823, 384, 338, and 682 nM for clothianidin, imidacloprid, thiamethoxam, and dinotefuran, respectively.

The broader implications of this work could be significant for those working in monitoring or mapping pesticides across entire watersheds/ecosystems as well as for those seeking to improve electrochemical electrode technology in general. The reported sensor circumvents the need to transport river water and soil samples to a lab for chromatographic analysis and allows for on-site testing. Hence this technology could permit rapid testing of agricultural runoffs and river water and help policy makers set guidelines on when and where such pesticides can be used. Furthermore, the creation of LIG electrochemical electrodes could be useful for a wide variety of applications that require high surface area and defect rich graphene for heterogenous charge transport such as with energy storage elements such as supercapacitors.

### Experimental

### Materials and Reagents

Phosphate buffer saline 10 mM potassium chloride pH 7.4, clothianidin, imidacloprid, thiamethoxam, dinotefuran, atrazine, glyphosate, 2,4-dichlorophenoxyacetic acid, dicamba, paraoxon, parathion, and fipronil were purchased from Sigma Aldrich. All pesticides were prepared in deionized water (18.4 MΩ-cm) except imidacloprid, clothianidin, and fipronil which were prepared in acetone and 100 times diluted in deionized water. Kapton polyimide film (125 nm) was purchased from DuPont.

### LIG Fabrication

A 75 W CO₂ Epilog Fusion M2 Laser was used to convert PI into graphene. A 5 mm diameter working electrode was lased using speed 15% (271.9 mm/s), power 7% (5.25 W), 1200 dots per inch, and a defocus length of 2 mm. The contacts were covered with silver paste to mitigate wear on the sensor. Acrylic polish was used as a passivation layer to control the working electrode surface area.

### Electrochemical Analysis

Palmsens4 potentiostat was used for all electrochemical measurements. Square wave voltammetry was performed in a 5 mL volume of PBS pH 7.4. The potential was stepped from −0.6 to −1.5 V with a potential step of 5 mV, amplitude of 25 mV, and frequency of 25 Hz. A three-electrode setup was used: LIG working electrode, commercial Ag/AgCl reference electrode, and commercial platinum wire counter electrode. Square wave voltammetry was run 10 times in bare buffer to purge the buffer and electrode surface of any redox species that was electroactive in the potential window. Sensors were calibrated to each pesticide by then mixing the pesticide for 2 minutes at 300 rpm. Stirring was then turned off and the scan was performed. Sensors were washed 2 times with deionized water before each addition of pesticide. Sensors were disposed of after use. Interference pesticides were tested at 40 μM. Results were reported as a change in current from the initially recorded baseline. The combined neonicotinoid model was calibrated to the average responses of each neonicotinoid for the corresponding concentrations. The accuracy of the model was tested by mixing 10 μM each of thiamethoxam, dinotefuran, clothianidin, and imidacloprid. This served as a model for 40 μM of neonicotinoid solution. Additionally, sensors were tested in a solution of river water brought to 0.5 M NaCl. Percent accuracy was reported for 40 μM concentration spikes compared to the current response found in 0.5 M NaCl deionized water which was used as the standard.

### References (for Specific Example 5 Section Supra)

1. Bonner, M. R. & Alavanj a, M. C. R. Pesticides, human health, and food security. Food Energy Secur. 6, 89-93 (2017).
2. Simon-Delso, N. et al. Systemic insecticides (Neonicotinoids and fipronil): Trends, uses, mode of action and metabolites. Environ. Sci. Pollut. Res. 22, 5-34 (2015).
3. Gibbons, D., Morrissey, C. & Mineau, P. A review of the direct and indirect effects of neonicotinoids and fipronil on vertebrate wildlife. Environ. Sci. Pollut. Res. 22, 103-118 (2015).
4. Morrissey, C. A. et al. Neonicotinoid contamination of global surface waters and associated risk to aquatic invertebrates: A review. Environment International vol. 74 291-303 (2015).
5. Bonmatin, J. M. et al. Environmental fate and exposure; neonicotinoids and fipronil. Environ. Sci. Pollut. Res. 22, 35-67 (2015).
6. Van Der Sluijs, J. P. et al. Conclusions of the worldwide integrated assessment on the risks of neonicotinoids and fipronil to biodiversity and ecosystem functioning. Environmental Science and Pollution Research vol. 22 148-154 (2015).
7 Christen, V., Schirrmann, M., Frey, J. E. & Fent, K. Global Transcriptomic Effects of Environmentally Relevant Concentrations of the Neonicotinoids Clothianidin, Imidacloprid, and Thiamethoxam in the Brain of Honey Bees (Apis mellifera). Environ. Sci. Technol. 52, 7534-7544 (2018).
8. Decio, P. et al. Acute thiamethoxam toxicity in honeybees is not enhanced by common fungicide and herbicide and lacks stress-induced changes in mRNA splicing. Sci. Rep. 9, 1-10 (2019).
9. Gregorc, A. et al. Effects of coumaphos and imidacloprid on honey bee (Hymenoptera: Apidae) lifespan and antioxidant gene regulations in laboratory experiments. Sci. Rep. 8, 1-13 (2018).
10. Chen, Z. et al. Ecological toxicity reduction of dinotefuran to honeybee: New perspective from an enantiomeric level. Environ. Int. 130, 104854 (2019).
11. Zhang, Y. et al. A Flexible Acetylcholinesterase-Modified Graphene for Chiral Pesticide Sensor. J. Am. Chem. Soc. 141, 14643-14649 (2019).
12. Ye, R., James, D. K. & Tour, J. M. Laser-Induced Graphene. Acc. Chem. Res. 51, 1609-1620 (2018).
13. Stanford, M. G. et al. High-Resolution Laser-Induced Graphene. Flexible Electronics beyond the Visible Limit. ACS Appl. Mater. Interfaces 12, 10902-10907 (2020).
14. Huang, L., Su, J., Song, Y. & Ye, R. Laser-Induced Graphene: En Route to Smart Sensing. Nano-Micro Lett. 12, 1-17 (2020).
15. Marques, A. C., Cardoso, A. R., Martins, R., Sales, M. G. F. & Fortunato, E. Laser-Induced Graphene-Based Platforms for Dual Biorecognition of Molecules. ACS Appl. Nano Mater. 3, 2795-2803 (2020).
16. Yao, Y. et al. The development of a novel biosensor based on gold nanocages/graphene oxide-chitosan modified acetylcholinesterase for organophosphorus pesticide detection. New J. Chem. 43, 13816-13826 (2019).
17. Zhang, J. et al. An acetylcholinesterase biosensor with high stability and sensitivity based on silver nanowire-graphene-TiO2 for the detection of organophosphate pesticides. RSC Adv. 9, 25248-25256 (2019).
18. Mahmoudi, E., Fakhri, H., Hajian, A., Afkhami, A. & Bagheri, H. High-performance electrochemical enzyme sensor for organophosphate pesticide detection using modified metal-organic framework sensing platforms. Bioelectrochemistry 130, 107348 (2019).
19. Zhao, F. et al. Smart plant-wearable biosensor for in-situ pesticide analysis. Biosens. Bioelectron. 170, 112636 (2020).
20. Pundir, C. S. & Chauhan, N. Acetylcholinesterase inhibition-based biosensors for pesticide determination: A review. Analytical Biochemistry vol. 429 19-31 (2012).
21. Dhara, K., Ramachandran, T., Nair, B. G. & Satheesh Babu, T. G. Single step synthesis of Au—CuO nanoparticles decorated reduced graphene oxide for high performance disposable nonenzymatic glucose sensor. J. Electroanal. Chem. 743, 1-9 (2015).
22. Xie, Y. et al. CuO nanoparticles decorated 3D graphene nanocomposite as non-enzymatic electrochemical sensing platform for malathion detection. J. Electroanal. Chem. 812, 82-89 (2018).
23. Rhouati, A., Majdinasab, M. & Hayat, A. A perspective on non-enzymatic electrochemical nanosensors for direct detection of pesticides. Current Opinion in Electrochemistry vol. 11 12-18 (2018).
24. Square Wave Voltammetry—an overview|ScienceDirect Topics. https://www.sciencedirect.com/topics/chemistry/square-wave-voltammetry.
25. Urbanová, V., Bakandritsos, A., Jakubec, P., Szambó, T. & Zbořil, R. A facile graphene oxide based sensor for electrochemical detection of neonicotinoids. Biosens. Bioelectron. 89, 532-537 (2017).
26. Öndeş, B. & Muti, M. Electrochemical Determination of Neonicotinoid Insecticide Clothianidin by Nanomaterial based Disposable Electrode. Anal. Bioanal. Electrochem vol. 12 www.abechem.com (2020).
27. Wolfram, J., Stehle, S., Bub, S., Petschick, L. L. & Schulz, R. Meta-Analysis of Insecticides in United States Surface Waters: Status and Future Implications. Environ. Sci. Technol. 52, 14452-14460 (2018).

### D. Options and Alternatives

Those of skill in this technical field will appreciate that the foregoing embodiments are not limiting. The invention can take a variety of forms.

One example is that variations to the disclosed embodiments and examples are possible, as would be appreciated and within the skill of those skilled in this technical art.

Other variations are possible. Below are a few:

1. Laser.

Lasers are featured in the above embodiments. The inventors have data to show UV lasers can be used to convert sp² carbon into sp² carbon to produce graphene. The quality of the graphene produced will depend on the laser used and the power density (J cm⁻²) delivered to the film. CO₂ lasers have shown to be better than UV to produce LIG.

2. Laser Operating Parameters.

The operating parameters that work for that Laser for the LIG patterns, functionalization, and pathogens in the specific embodiments above can vary. They can vary from the indicated states or settings even though they may not produce optimal results. They can still produce effective results, which is intended to mean they can detect an analyte with sufficient accuracy, repeatability, and precision as to be useful for a given application. This includes, but is not limited to, effectiveness at least on the order of effectiveness of at least some state-of-the-art electrochemical or bio-sensors of the types discussed herein. There is a range of operating parameters that would produce effective results in this sense. It is envisioned that a range of laser power densities (J cm⁻²) delivered at polyimide film that would be effective to produce LIG-based patterns. This information would be universal for different lasers (e.g., UV and CO₂ laser). As a rule, the settings are primarily dependent on the laser type, i.e., for CO₂ laser the variables are focal lens offset [mm], laser speed [cm s⁻¹], and laser power [W].

3. Laser Beam and its Optics.

In the specific examples, a lens is typically used with the with the laser. The optics used with the laser can vary according to need or desire. A main requirement for the embodiments is a converging lens. Multiple size lenses can be used, but each lens will have an effect on the required lens offset [mm], laser speed [cm s⁻¹], and laser power [W]. Currently, we are determining the upper and lower limit of the beam diameter. The theoretical limit of LIG line width is the same as the smallest beam diameter possible.

4. LIG.

At least in the exemplary embodiments LIG can be accomplished in one pass of the laser. One pass of the laser can formulate LIG on a polyimide surface. Multiple passes can be used to produce LIG, also when there is a need to change the hydrophobicity of LIG produced with one pass. LIG formation is determined by combination of characterization results. First electrically (e.g., the use of a multimeter and sheet resistance measurements); second, electrochemically (e.g., cyclic voltammetry and electrochemical impedance spectroscopy); third, spectroscopic results (e.g., Raman spectroscopy and X-ray photoelectron spectroscopy).

5. Substrate from which LIG can be Generated.

The primary example of a substrate from which LIG can be produced is polyimide. Other groups have demonstrated that any carbon precursor that can be converted into amorphous carbon can be converted into LIG upon further treatment with a CO₂ or UV laser. Some examples include polysulfones, poly(ether imide), and polyphenylene sulfide. In general, some level of temperature resistance is desired along with a prerequisite of a cyclic carbon structure. Lower quality LIG have been shown in dried coffee grind, coconut shell, and dried wood, among others.

6. Functionalizations

Use of electrodes according to the present invention can be implemented in a variety of ways. Non-limiting examples of different functionalizations or applications with which electrodes according to one or more aspects of the invention have been discussed supra. As will be appreciated by those skilled in this technical art, variations on those are, of course possible, according to need or desire.

Background information regarding some of the applications can be found at the following, each of which is incorporated by reference herein:

US20200025753A1 to Claussen et al. discusses the basics of electrode-based immunosensors.

US2019/0088420A1 to Tours, et al. discusses basics of electrode-based water splitting and energy harvesting.

U.S. Pat. No. 10,900,925B2 to Chumbimuni-Torres et al. discusses basics of electrode-based ion selective sensing.

The foregoing are for example and not limitation.

What is claimed is:

1. An electrode comprising:
a. a working area;
b. at least one electrical connection for operatively connecting the working area to an electrical circuit;
c. the working area comprising a laser-induced graphene (LIG) pattern comprising:
i. a porous, multi-layered, turbostratic structure graphene for effective for heterogenous charge transport; and
ii. the highly porous graphene functionalized for application as one of:
1. an electrode-based biochemical sensor with a biorecoginition agent;
2. an electrode-based ion selective sensor with an ionophore;
3. an electrode-based pesticide monitor;
4. a plural electrode-based water splitter; or
5. an electrode-based pesticide detector free of a bio-recognition agent.

2. The electrode of claim 1 wherein the highly porous graphene from LIG comprises 3D structures which are:
a. rich in edge-planes pyrolytic graphite (EPPG); and
b. have microporous/mesoporous thickness of 15-20 μM, and
c. the highly porous graphene from LIG is made by controlling a laser relative to a carbon precursor to generate at least one of:
i. convert the carbon precursor into amorphous graphene or graphitic carbon;
ii. convert sp³ carbon into sp² carbon by photothermal effects at surface (e.g., >1000 degrees C.); and
iii. ablate the carbon to provide a carbon frame organized into long-range ordered graphene layers.

3. The electrode of claim 1 wherein the laser is defocused from or out of plane of the substrate surface during operation.

4. The electrode of claim 2 wherein the carbon precursor comprises one of:
a. polyimide;
b. polysulfone;
c. poly(ether imide); and
d. polyphenylene sulfide.

5. The electrode of claim 1 wherein the electrode-based biochemical comprises an immunosensor, the working area is functionalized with a biorecognition agent the biorecognition agent comprises an antibody, and the target chemical species of interest comprises an antigen.

6. The electrode of claim 5 wherein the antigen comprises a pathogen.

7. The electrode of claim 6 wherein the pathogen comprises one of:
a. Salmonella enterica;
b. Escherichia coli;
c. Listeria monocytogenes;
d. Staphylococcus aureus;
e. Bacillus cereus; or
f. Pseudomonas aeruginosa.

8. The electrode of claim 5 in operative connection to an immunosensor transducer and readout system.

9. The electrode of claim 1 wherein the electrode-based ion selective sensor comprises a solid state ion-selective sensor, the working area is functionalized with the ionophore, and the ionophore is added to the working area of the electrode in ion-selective membrane form.

10. The electrode of claim 9 wherein the ionophore comprises K⁺ and/or H⁺.

11. The electrode of claim 9 in operative connection to an ion selective transducer and readout system.

12. The electrode of claim 1 wherein the electrode-based pesticide monitor working area is functionalized with an enzyme sensitive to a pesticide of interest.

13. The electrode of claim 12 wherein the enzyme comprises horseradish peroxidase.

14. The electrode of claim 13 wherein the pesticide of interest comprises one of:
a. glyphosate;
b. atrazine; and
c. dichlofenthion.

15. The electrode of claim 14 in operative connection to a potentiometric or impedimetric transducer and readout system.

16. The electrode of claim 1 wherein the electrode-based water splitter comprises:
a. a first said electrode with a working area lasered with a second pass; and
b. a second said electrode with a working area to which platinum (Pt) is applied.

17. The electrode of claim 16 wherein the first and second electrodes are in operative connection with a water splitting circuit and system.

18. The electrode of claim 16 used for energy harvesting.

19. The electrode of claim 1 functionalized for pesticide detection by:
a. a biorecognition-free working area;
b. an electrical connection spaced from the working area; and
c. a passivated area between the working area and the electrical connection.

20. The electrode of claim 19 wherein the pesticide is from the group comprising neonicotinoids, and the electrode is operatively connected to a potentiometric transducer and readout system.

21. A method of electrode-based operations comprising:
a. direct writing of a laser induced graphene (LIG) pattern;
b. functionalizing at least a portion of the LIG pattern adapted for one of:
i electrode-based biochemical sensing with a biorecoginition agent;
ii. electrode-based ion selective sensing with an ionophore;
iii. electrode-based pesticide monitoring;
iv. electrode-based water splitting; or
v. electrode-based pesticide detecting free of a bio-recognition agent
c. placing the electrode in operative position for the application; and
d. conducting impedimetric, potentiometric, or electric operations with the functionalized LIG pattern.

22. The method of claim 21 wherein the direct writing of the LIG pattern comprises controlling spatial position, focusing position, and power density (J cm⁻²) of a laser.

23. The method of claim 22 wherein the direct written LIG pattern comprises one of:
a. an active working area;
b. a sensing area and a passivated portion extending to an electrical connection;
c. an interdigitated electrode (IDE);
d. a dipstick electrode;
e. a serpentine electrode; or
f. an all-in-one electrode.

24. The method of claim 21 wherein the laser induction comprises controlling a laser relative to the porous graphene to create a LIG pattern at:
a. a material distance of on the order of 74 mm;
b. a beam size of on the order of 176 mm;
c. in ambient atmosphere;
d. with a laser whether focused or defocused;
e. by laser direct writing (LDW) which is:
i. maskless, catalyst free, non-toxic, controllable, and non-contact;
ii. with laser parameters comprising:
1. low power density (e.g., for CO₂ on the order of 60 W cm⁻²);
2. a relatively rapid exposure time (e.g., on the order of a few tens of minutes and not a few hours or days);
3. pulsed laser energy.

25. A method of making an economical, disposable, highly sensitive, rapid, in-field electrode comprising:
a. scanning a laser over a carbon-containing thin-film or sheet substrate to create a high porosity laser-induced graphene (LIG) pattern; and
b. functionalizing at least a portion of the high porosity LIG pattern for an application.

26. The method of claim 25 wherein the application comprises one of:
a. electrode-based biochemical sensing with a biorecoginition agent;
b. electrode-based ion selective sensing with an ionophore;
c. electrode-based pesticide monitoring;
d. plural electrode-based water splitting; or
electrode-based pesticide detecting free of a bio-recognition agent.
